SEQUENCE LISTING (51 sequences)

SEQ ID NO: 1 - DNA, 1666 bp, Lycopersicon esculentum
  CDS (1)..(1494)
atggaagctcttctcaagccttttccatctcttttactttcctctcctacaccccataggtctattttccaacaaaatccctcttttctaagtcccaccaccaaaaaaaaatcaagaaaatgtcttcttagaaacaaaagtagtaaacttttttgtagctttcttgatttagcacccacatcaaagccagagtctttagatgttaacatctcatgggttgatcctaattcgaatcgggctcaattcgacgtgatcattatcggagctggccctgctgggctcaggctagctgaacaagtttctaaatatggtattaaggtatgttgtgttgacccttcaccactctccatgtggccaaataattatggtgtttgggttgatgagtttgagaatttaggactggaaaattgtttagatcataaatggcctatgacttgtgtgcatataaatgataacaaaactaagtatttgggaagaccatatggtagagttagtagaaagaagctgaagttgaaattgttgaatagttgtgttgagaacagagtgaagttttataaagctaaggtttggaaagtggaacatgaagaatttgagtcttcaattgtttgtgatgatggtaagaagataagaggtagtttggttgtggatgcaagtggttttgctagtgattttatagagtatgacaggccaagaaaccatggttatcaaattgctcatggggttttagtagaagttgataatcatccatttgatttggataaaatggtgcttatggattggagggattctcatttgggtaatgagccatatttaagggtgaataatgctaaagaaccaacattcttgtatgcaatgccatttgatagagatttggttttcttggaagagacttctttggtgagtcgtcctgttttatcgtatatggaagtaaaaagaaggatggtggcaagattaaggcatttggggatcaaagtgaaaagtgttattgaggaagagaaatgtgtgatccctatgggaggaccacttccgcggattcctcaaaatgttatggctattggtgggaattcagggatagttcatccatcaacagggtacatggtggctaggagcatggctttagcaccagtactagctgaagccatcgtcgaggggcttggctcaacaagaatgataagagggtctcaactttaccatagagtttggaatggtttgtggcctttggatagaagatgtgttagagaatgttattcatttgggatggagacattgttgaagcttgatttgaaagggactaggagattgtttgacgctttctttgatcttgatcctaaatactggcaagggttcctttcttcaagattgtctgtcaaagaacttggtttactcagcttgtgtcttttcggacatggctcaaacatgactaggttggatattgttacaaaatgtcctcttcctttggttagactgattggcaatctagcaatagagagcctttgaatgtgaaaagtttgaatcattttcttcattttaatttctttgattattttcatattttctcaattgcaaaagtgagataagagctacatactgtcaacaaataaactactattggaaagttaaaatatgtgtttgttgtatgttattctaatggaatggattttgtaaa

SEQ ID NO: 2 - protein, 498 aa, Lycopersicon esculentum
MEALLKPFPSLLLSSPTPHRSIFQQNPSFLSPTTKKKSRKCLLRNKSSKLFCSFLDLAPTSKPESLDVNISWVDPNSNRAQFDVIIIGAGPAGLRLAEQVSKYGIKVCCVDPSPLSMWPNNYGVWVDEFENLGLENCLDHKWPMTCVHINDNKTKYLGRPYGRVSRKKLKLKLLNSCVENRVKFYKAKVWKVEHEEFESSIVCDDGKKIRGSLVVDASGFASDFIEYDRPRNHGYQIAHGVLVEVDNHPFDLDKMVLMDWRDSHLGNEPYLRVNNAKEPTFLYAMPFDRDLVFLEETSLVSRPVLSYMEVKRRMVARLRHLGIKVKSVIEEEKCVIPMGGPLPRIPQNVMAIGGNSGIVHPSTGYMVARSMALAPVLAEAIVEGLGSTRMIRGSQLYHRVWNGLWPLDRRCVRECYSFGMETLLKLDLKGTRRLFDAFFDLDPKYWQGFLSSRLSVKELGLLSLCLFGHGSNMTRLDIVTKCPLPLVRLIGNLAIESL

SEQ ID NO: 3 - DNA, 1608 bp, Haematococcus pluvialis
  CDS (3)..(971)
ctacatttcacaagcccgtgagcggtgcaagcgctctgccccacatcggcccacctcctcatctccatcggtcatttgctgctaccacgatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagggtgcggaaccaggcacgctggtttcacacctcatgcctgtgataaggtgtggctagagcgatgcgtgtgagacgggtatgtcacggtcgactggtctgatggccaatggcatcggccatgtctggtcatcacgggctggttgcctgggtgaaggtgatgcacatcatcatgtgcggttggaggggctggcacagtgtgggctgaactggagcagttgtccaggctggcgttgaatcagtgagggtttgtgattggcggttgtgaagcaatgactccgcccatattctatttgtgggagctgagatgatggcatgcttgggatgtgcatggatcatggtagtgcagcaaactatattcacctagggctgttggtaggatcaggtgaggccttgcacattgcatgatgtactcgtcatggtgtgttggtgagaggatggatgtggatggatgtgtattctcagacgtagaccttgactggaggcttgatcgagagagtgggccgtattctttgagaggggaggctcgtgccagaaatggtgagtggatgactgtgacgctgtacattgcaggcaggtgagatgcactgtctcgattgtaaaatacattcagatgcaaaaaaaaaaaaaaaaaaaaaaa

SEQ ID NO: 4 - protein, 322 aa, Haematococcus pluvialis
TFHKPVSGASALPHIGPPPHLHRSFAATTMLSKLQSISVKARRVELARDITRPKVCLHAQRCSLVRLRVAAPQTEEALGTVQAAGAGDEHSADVALQQLDRAIAERRARRKREQLSYQAAAIAASIGVSGIAIFATYLRFAMHMTVGGAVPWGEVAGTLLLVVGGALGMEMYARYAHKAIWHESPLGWLLHKSHHTPRTGPFEANDLFAIINGLPAMLLCTFGFWLPNVLGAACFGAGLGITLYGMAYMFVHDGLVHRRFPTGPIAGLPYMKRLTVAHQLHHSGKYGGAPWGMFLGPQELQHIPGAAEEVERLVLELDWSKR

SEQ ID NO: 5 - DNA, 1125 bp, Lycopersicon esculentum
  CDS (20)..(946)
ttggtcatctccacaatcaatggctgccgccgccagaatctccgcctcctctacctcacgaactttttatttccgtcattcaccgtttcttggcccaaaacctacttcgacaacctcacatgtttctccaatctctcctttttctcttaatctaggcccaattttgaggtctagaagaaaacccagtttcactgtttgctttgttctcgaggatgagaagctgaaacctcaatttgacgatgaggctgaggattttgaaaagaagattgaggaacagatcttagctactcgcttggcggagaaactggctaggaagaaatcggagaggtttacttatcttgtggctgctataatgtctagttttgggattacttctatggctgttatggctgtttattacagattttcgtggcaaatggagggaggagaagttcctgtaaccgaaatgttgggtacatttgctctctctgttggtgctgctgtaggaatggagttttgggcgagatgggcacacaaagcactgtggcatgcttcactatggcacatgcatgagtcacaccacaaaccaagagaaggaccttttgagctgaacgacgttttcgccataacaaacgctgttccagcaatagccctcctcaactatggtttcttccataaaggcctcattgccggactatgcttcggtgctgggctagggatcacagtatttggaatggcatacatgtttgttcacgatggtttggttcacaagagattcccagttggacctgtagccaatgtaccttatcttaggaaggtggctgctgctcattcgcttcatcactcagagaagttcaatggtgtcccatatggcttgttcttcggacctaaggaactggaagaagtaggagggacggaagagttggaaaaggaagtgatacgaaggacgagactttcgaaaggatcatgaacgattgttcataaacatagaatgtcattttacacttcttatcaatgaggaagggtgatttttgatgtatttgatagtagagaaaaatgtagctctcttgatgaaatgaatttgtatttatgtaggctcttcttattcagtaagattttttcttttttttgatctcgtgccgaatt

SEQ ID NO: 6 - protein, 309 aa, Lycopersicon esculentum
MAAAARISASSTSRTFYFRHSPFLGPKPTSTTSHVSPISPFSLNLGPILRSRRKPSFTVCFVLEDEKLKPQFDDEAEDFEKKIEEQILATRLAEKLARKKSERFTYLVAAIMSSFGITSMAVMAVYYRFSWQMEGGEVPVTEMLGTFALSVGAAVGMEFWARWAHKALWHASLWHMHESHHKPREGPFELNDVFAITNAVPAIALLNYGFFHKGLIAGLCFGAGLGITVFGMAYMFVHDGLVHKRFPVGPVANVPYLRKVAAAHSLHHSEKFNGVPYGLFFGPKELEEVGGTEELEKEVIRRTRLSKGS

SEQ ID NO: 7 - DNA, 1830 bp, Tagetes erecta
  CDS (141)..(1691)
ggcacgaggcaaagcaaaggttgtttgttgttgttgttgagagacactccaatccaaacagatacaaggcgtgactggatatttctctctcgttcctaacaacagcaacgaagaagaaaaagaatcattactaacaatcaatgagtatgagagctggacacatgacggcaacaatggcggcttttacatgccctaggtttatgactagcatcagatacacgaagcaaattaagtgcaacgctgctaaaagccagctagtcgttaaacaagagattgaggaggaagaagattatgtgaaagccggtggatcggagctgctttttgttcaaatgcaacagaataagtccatggatgcacagtctagcctatcccaaaagctcccaagggtaccaataggaggaggaggagacagtaactgtatactggatttggttgtaattggttgtggtcctgctggccttgctcttgctggagaatcagccaagctaggcttgaatgtcgcacttatcggccctgatcttccttttacaaataactatggtgtttgggaggatgaatttataggtcttggacttgagggctgtattgaacatgtttggcgagatactgtagtatatcttgatgacaacgatcccattctcataggtcgtgcctatggacgagttagtcgtgatttacttcacgaggagttgttgactaggtgcatggagtcaggcgtttcatatctgagctccaaagtggaacggattactgaagctccaaatggcctaagtctcatagagtgtgaaggcaatatcacaattccatgcaggcttgctactgtcgcttctggagcagcttctggaaaacttttgcagtatgaacttggcggtccccgtgtttgcgttcaaacagcttatggtatagaggttgaggttgaaagcataccctatgatccaagcctaatggttttcatggattatagagactacaccaaacataaatctcaatcactagaagcacaatatccaacatttttgtatgtcatgccaatgtctccaactaaagtattctttgaggaaacttgtttggcttcaaaagaggccatgccttttgagttattgaagacaaaactcatgtcaagattaaagactatggggatccgaataaccaaaacttatgaagaggaatggtcatatattccagtaggtggatccttaccaaataccgagcaaaagaaccttgcatttggtgctgctgctagcatggtgcatccagccacaggatattcggttgtaagatcactgtcagaagctcctaattatgcagcagtaattgcaaagattttagggaaaggaaattcaaaacagatgcttgatcatggaagatacacaaccaacatctcaaagcaagcttgggaaacactttggccccttgaaaggaaaagacagagagcattctttctctttggattagcactgattgtccagatggatattgaggggacccgcacattcttccggactttcttccgcttgcccacatggatgtggtgggggtttcttggatcttcgttatcatcaactgacttgataatatttgcgttttacatgtttatcatagcaccgcatagcctgagaatgggtctggttagacatttgctttctgacccgacaggaggaacaatgttaaaagcgtatctcacgatataaataactctagtcgcgatcagtttagattataggcacatcttgcatatatatatgtataaaccttatgtgtgctgtatccttacatcaacacagtcattaattgtatttcttggggtaatgctgatgaagtattttctgg

SEQ ID NO: 8 - protein, 516 aa, Tagetes erecta
MSMRAGHMTATMAAFTCPRFMTSIRYTKQIKCNAAKSQLVVKQEIEEEEDYVKAGGSELLFVQMQQNKSMDAQSSLSQKLPRVPIGGGGDSNCILDLVVIGCGPAGLALAGESAKLGLNVALIGPDLPFTNNYGVWEDEFIGLGLEGCIEHVWRDTVVYLDDNDPILIGRAYGRVSRDLLHEELLTRCMESGVSYLSSKVERITEAPNGLSLIECEGNITIPCRLATVASGAASGKLLQYELGGPRVCVQTAYGIEVEVESIPYDPSLMVFMDYRDYTKHKSQSLEAQYPTFLYVMPMSPTKVFFEETCLASKEAMPFELLKTKLMSRLKTMGIRITKTYEEEWSYIPVGGSLPNTEQKNLAFGAAASMVHPATGYSVVRSLSEAPNYAAVIAKILGKGNSKQMLDHGRYTTNISKQAWETLWPLERKRQRAFFLFGLALIVQMDIEGTRTFFRTFFRLPTWMWWGFLGSSLSSTDLIIFAFYMFIIAPHSLRMGLVRHLLSDPTGGTMLKAYLTI

SEQ ID NO: 9 - DNA, 358 bp, Tagetes erecta
  Promoter: Sense promoter
aagcttaccgatagtaaaatcgttagttatgattaatacttgggaggtgggggattataggctttgttgtgagaatgttgagaaagaggtttgacaaatcggtgtttgaatgaggttaaatggagtttaattaaaataaagagaagagaaagattaagagggtgatggggatattaaagacggccaatatagtgatgccacgtagaaaaaggtaagtgaaaacatacaacgtggctttaaaagatggcttggctgctaatcaactcaactcaactcatatcctatccattcaaattcaattcaattctattgaatgcaaagcaaagcaaagcaaaggttgtttgttgttgttgtcgac

SEQ ID NO: 10 - DNA, 445 bp, Tagetes erecta
  misc_feature: Sense fragment
aagcttgcacgaggcaaagcaaaggttgtttgttgttgttgttgagagacactccaatccaaacagatacaaggcgtgactggatatttctctctcgttcctaacaacagcaacgaagaagaaaaagaatcattactaacaatcaatgagtatgagagctggacacatgacggcaacaatggcggcttttacatgccctaggtttatgactagcatcagatacacgaagcaaattaagtgcaacgctgctaaaagccagctagtcgttaaacaagagattgaggaggaagaagattatgtgaaagccggtggatcggagctgctttttgttcaaatgcaacagaataagtccatggatgcacagtctagcctatcccaaaagctcccaagggtaccaataggaggaggaggagacagtaactgtatactggatttggttgtcgac

SEQ ID NO: 11 - DNA, 446 bp, Tagetes erecta
  misc_feature: Antisense fragment
gaattcgcacgaggcaaagcaaaggttgtttgttgttgttgttgagagacactccaatccaaacagatacaaggcgtgactggatatttctctctcgttcctaacaacagcaacgaagaagaaaaagaatcattactaacaatcaatgagtatgagagctggacacatgacggcaacaatggcggcttttacatgccctaggtttatgactagcatcagatacacgaagcaaattaagtgcaacgctgctaaaagccagctagtcgttaaacaagagattgaggaggaagaagattatgtgaaagccggtggatcggagctgctttttgttcaaatgcaacagaataagtccatggatgcacagtctagcctatcccaaaagctcccaagggtaccaataggaggaggaggagacagtaactgtatactggatttggttggatcct

SEQ ID NO: 12 - DNA, 393 bp, Tagetes erecta
  misc_feature: Sense fragment
aagctttggattagcactgattgtccagatggatattgaggggacccgcacattcttccggactttcttccgcttgcccacatggatgtggtgggggtttcttggatcttcgttatcatcaactgacttgataatatttgcgttttacatgtttatcatagcaccgcatagcctgagaatgggtctggttagacatttgctttctgacccgacaggaggaacaatgttaaaagcgtatctcacgatataaataactctagtcgcgatcagtttagattataggcacatcttgcatatatatatgtataaaccttatgtgtgctgtatccttacatcaacacagtcattaattgtatttcttggggtaatgctgatgaagtattttctgtcgac

SEQ ID NO: 13 - DNA, 397 bp, Tagetes erecta
  misc_feature: Antisense fragment
gaattctctttggattagcactgattgtccagatggatattgaggggacccgcacattcttccggactttcttccgcttgcccacatggatgtggtgggggtttcttggatcttcgttatcatcaactgacttgataatatttgcgttttacatgtttatcatagcaccgcatagcctgagaatgggtctggttagacatttgctttctgacccgacaggaggaacaatgttaaaagcgtatctcacgatataaataactctagtcgcgatcagtttagattataggcacatcttgcatatatatatgtataaaccttatgtgtgctgtatccttacatcaacacagtcattaattgtatttcttggggtaatgctgatgaagtattttctggatcc

SEQ ID NO: 14 - DNA, 358 bp, Tagetes erecta; identical to SEQ ID NO: 9
  Promoter: Sense promoter

SEQ ID NO: 15 - DNA, 361 bp, Tagetes erecta
  promoter: Antisense promoter
ctcgagcttaccgatagtaaaatcgttagttatgattaatacttgggaggtgggggattataggctttgttgtgagaatgttgagaaagaggtttgacaaatcggtgtttgaatgaggttaaatggagtttaattaaaataaagagaagagaaagattaagagggtgatggggatattaaagacggccaatatagtgatgccacgtagaaaaaggtaagtgaaaacatacaacgtggctttaaaagatggcttggctgctaatcaactcaactcaactcatatcctatccattcaaattcaattcaattctattgaatgcaaagcaaagcaaagcaaaggttgtttgttgttgttggatcc

SEQ ID NO: 16 - DNA, 332 bp, Tagetes erecta
  CDS (1)..(330)
aagcttgcacgagcctctctctatttttacacttcaatggcggcagcaattgctgtcccttgtagctcaagaccatttggcttaggtcgaatgcggttacttggtcataaacccacaaccataacttgtcacttccccttttctttttctatcaaatcatttaccccaattgttaggggcagaagatgtactgtttgttttgttgccggtggcgacagtaatagtaacagtaataataatagtgacagtaatagtaataatccgggtctggatttaaacccggcggttatgaaccgtaaccgtttggttgaagaaaaaatggagaggtcgac

SEQ ID NO: 17 - protein, 110 aa, Tagetes erecta
KLARASLYFYTSMAAAIAVPCSSRPFGLGRMRLLGHKPTTITCHFPFSFSIKSFTPIVRGRRCTVCFVAGGDSNSNSNNNSDSNSNNPGLDLNPAVMNRNRLVEEKMERS

SEQ ID NO: 18 - DNA, 332 bp, Tagetes erceta; identical to SEQ ID NO: 16
  misc_feature: beta-Hydroxylase sense fragment

SEQ ID NO: 19 - DNA, 332 bp, Tagetes erecta
  misc_feature: beta-Hydroxylase antisense fragment
gaattcggcacgagcctctctctatttttacacttcaatggcggcagcaattgctgtcccttgtagctcaagaccatttggcttaggtcgaatgcggttacttggtcataaacccacaaccataacttgtcacttccccttttctttttctatcaaatcatttaccccaattgttaggggcagaagatgtactgtttgttttgttgccggtggcgacagtaatagtaacagtaataataatagtgacagtaatagtaataatccgggtctggatttaaacccggcggttatgaaccgtaaccgtttggttgaagaaaaaatggagaggatcc

SEQ ID NO: 20 - DNA, 19 bp, Artificial sequence
  Primer
tgccaaagtaactctttat

SEQ ID NO: 21 - DNA, 19 bp, Artificial sequence
  Primer
aggtgcatgaccaagtaac

SEQ ID NO: 22 - DNA, 1033 bp, Lycopersicon esculentum
  Promoter
aggtgcatgaccaagtaacaatttgattcctttccagcataacgtcatgttggttgcaaaaagaaggcaaagtagagcaagcaagcaagcaaagcatttttcttattttatattttgttgcggattccaccacccacttgaaaaattgacatgtcacaatgatttcgtatcctagtcttttattatttaacactctcacaatcccattactctacacctctttcattaagtcaacacacggttttcaaaaatccactaccctcccaccacctagaatcttttgttacctaccaacaccctcctttgttctctttatatattggtccaactaaatcaataagggaaagcatccttttggttggaggaattgctttcattctcactctttgtgtgttgatcaatggactagctaataacaagttcctcctctatatatttcaaaagaatggaacagaaacataaacgaaagacagagtacctgatgttgatgattcattgtctgtctggagctcccaaatgccttttatgcttacatattcataaccaacaacggctattaattataaaccaaaaacacgaaataagtttgtagcaaagtgaaattaggaatcttggagatggatccattagtagtaggataataggatatgatggaatttggttggggaacagtgataacttacgcttgcttccggcgccgggaaagttggaaaacctacaaagtacagaaatggatctgggccttgaagtgggctttttattaaagaaaaaaatacatctccgttatcaatcaccatcttcttctatctacaaattaaagaaggtaacaacagaacgtggtggatcatgtggttaggcattaattatttgctttgtttcgccgttttggtaacacacagacacagttccggtaagagcttttgcagccactctttatagttatttagaattggcgatcgaatcaatctcactccctccctcccttaagtcttgttgaatctgctgaattgttttataaagagttactttggca

SEQ ID NO: 23 - DNA, 18 bp, Artificial sequence
  Primer
atggaagctcttctcaag

SEQ ID NO: 24 - DNA, 22 bp, Artificial sequence
  Primer
ctattgctagattgccaatcag

SEQ ID NO: 25 - DNA, 28 bp, Artificial sequence
  Primer
gagctcactcactgatttccattgcttg

SEQ ID NO: 26 - DNA, 37 bp, Artificial sequence
  Primer
cgccgttaagtcgatgtccgttgatttaaacagtgtc

SEQ ID NO: 27 - DNA, 34 bp, Artificial sequence
  Primer
atcaacggacatcgacttaacggcgtttgtaaac

SEQ ID NO: 28 - DNA, 25 bp, Artificial sequence
  Primer
taagctttttgttgaagagatttgg

SEQ ID NO: 29 - DNA, 23 bp, Artificial sequence
  Primer
gaaaatacttcatcagcattacc

SEQ ID NO: 30 - DNA, 28 bp, Artificial sequence
  Primer
gtcgactacgtaagtttctgcttctacc

SEQ ID NO: 31 - DNA, 26 bp, Artificial sequence
  Primer
ggatccggtgatacctgcacatcaac

SEQ ID NO: 32 - DNA, 28 bp, Artificial sequence
  Primer
aagcttgcacgaggcaaagcaaaggttg

SEQ ID NO: 33 - DNA, 29 bp, Artificial sequence
  Primer
gtcgacaaccaaatccagtatacagttac

SEQ ID NO: 34 - DNA, 30 bp, Artificial sequence
  Primer
aggatccaaccaaatccagtatacagttac

SEQ ID NO: 35 - DNA, 28 bp, Artificial sequence
  Primer
gaattcgcacgaggcaaagcaaaggttg

SEQ ID NO: 36 - DNA, 25 bp, Artificial sequence
  Primer
aagctttggattagcactgattgtc

SEQ ID NO: 37 - DNA, 29 bp, Artificial sequence
  Primer
gtcgacagaaaatacttcatcagcattac

SEQ ID NO: 38 - DNA, 29 bp, Artificial sequence
  Primer
ggatccagaaaatacttcatcagcattac

SEQ ID NO: 39 - DNA, 27 bp, Artificial sequence
  Primer
gaattctctttggattagcactgattg

SEQ ID NO: 40 - DNA, 23 bp, Artificial sequence
  Primer
cgccttgtatctgtttggattgg

SEQ ID NO: 41 - DNA, 24 bp, Artificial sequence
  Primer
ctaacaatcaatgagtatgagagc

SEQ ID NO: 42 - DNA, 26 bp, Artificial sequence
  Primer
agagcaaggccagcaggaccacaacc

SEQ ID NO: 43 - DNA, 26 bp, Artificial sequence
  Primer
ccttgggagcttttgggataggctag

SEQ ID NO: 44 - DNA, 26 bp, Artificial sequence
  Primer
tcacgccttgtatctgtttggattgg

SEQ ID NO: 45 - DNA, 15 bp, Artificial sequence
  Primer
gtcgagtatggagtt

SEQ ID NO: 46 - DNA, 28 bp, Artificial sequence
  Primer
aagcttaccgatagtaaaatcgttagtt

SEQ ID NO: 47 - DNA, 31 bp, Artificial sequence
  Primer
ctcgagcttaccgatagtaaaatcgttagtt

SEQ ID NO: 48 - DNA, 28 bp, Artificial sequence
  Primer
gtcgacaacaacaacaaacaacctttgc

SEQ ID NO: 49 - DNA, 28 bp, Artificial sequence
  Primer
ggatccaacaacaacaaacaacctttgc

SEQ ID NO: 50 - DNA, 22 bp, Artificial sequence
  Primer
gagctctacaaattagggttac

SEQ ID NO: 51 - DNA, 23 bp, Artificial sequence
  Primer
aagcttattatttccaaattccg

(19) United States
(12) Patent Application Publication
(10) Pub. No.: US 2006/0059584 A1
(43) Pub. Date: Mar. 16, 2006

(54) Method for the production of $g(b)-carotinoids

(75) Inventors: Martin Klebsattel, Quedlinburg (DE); Matt Sauer, Quedlinburg (DE); Ralf Flachmann, Quedlinburg (DE); Christel Renate Schopfer, Quedlinburg (DE)

Correspondence Address: CONNOLLY BOVE LODGE & HUTZ, LLP, P O BOX 2207, WILMINGTON, DE, 19899, US

(73) Assignee: SunGene GmbH & Co. KGaA, Gatersleben (DE)

(21) Appl. No.: 10/524,971

(22) PCT Filed: Aug. 18, 2003
(86) PCT No.: PCT/EP03/09101

§ 371 Date: Feb. 18, 2005

(30) Foreign Application Priority Data
Aug. 20, 2002 (DE) 102 38 980.2
Aug. 20, 2002 (DE) 102 38 979.9
Dec. 16, 2002 (DE) 102 38 971.2

(51) Int. Cl. A01H 1/00; C12P 23/00; C12N 15/82; C12N 5/04

(52) U.S. Cl. 800/282; 435/67; 435/419; 435/468

(57) ABSTRACT

The present invention relates to a process for the production of β-carotenoids by culturing genetically modified plants, the genetically modified plants, and their use as foodstuffs and feedstuffs and for the production of β-carotenoid extracts.

The present invention relates to a process for the production of β-carotenoids by culturing genetically modified plants, the genetically modified plants, and their use as foodstuffs and feedstuffs and for the production of β-carotenoid extracts.

Carotenoids are synthesized de novo in bacteria, algae, fungi and plants. β-Carotenoids, that is carotenoids of the β-carotene pathway, such as, for example, β-carotene, β-cryptoxanthin, zeaxanthin, antheraxanthin, violaxanthin and neoxanthin are natural antioxidants and pigments which are produced as secondary metabolites by microorganisms, algae, fungi and plants.

β-Carotene is a vitamin A precursor and thus an important constituent in food, feed and cosmetic applications. It further serves as a pigmenting substance in many fields, such as, for example, in the beverages industry.

Zeaxanthin is one of the main pigments in the macula of the human eye and protects the sensitive visual cells by means of its special light absorption spectrum. Zeaxanthin is degraded by light irradiation and must be administered again with the food in order to obtain efficient protection of the macula and to avoid long-term damage, such as age-related macular degeneration (AMD). Zeaxanthin further serves as a substance for pigmenting animal products, in particular for the pigmentation of egg yolks, skin and meat of game birds by oral administration.

Many β-carotenoids are moreover of high economic interest, since in their property as color pigments and antioxidants they can be utilized as foodstuff additives, dyes, preservatives, feedstuffs and food supplements.

The preparation of β-carotenoids, such as, for example, β-carotene and zeaxanthin nowadays for the most part takes place by chemical synthesis processes.

Natural β-carotenoids, such as, for example, natural β-carotene, are obtained in biotechnological processes in small amounts by culturing microorganisms, algae or fungi or by fermentation of microorganisms optimized by genetic engineering and subsequent isolation.

Natural zeaxanthin is a constituent of “oleoresin”, an extract of dried petals of the plant Tagetes erecta. The content of zeaxanthin in oleoresin is, however, low, since the carotenoids in the petals of Tagetes erecta consist to the vast majority of carotenoids of the α-carotene pathway, such as, for example, lutein.

The increase in the β-carotenoid content in plants or in the corresponding plant tissues is therefore an important aim of the biotechnological optimization of plants.

Carlo Rosati et al. describe the overexpression of a β-cyclase from Arabidopsis thaliana using a fruit-specific promoter in tomato (Rosati C, Aquilani R, Dharmapuri S, Pallara P, Marusic C, Tavazza R, Bouvier F, Camara B, Giuliano G. Metabolic engineering of beta-carotene and lycopene content in tomato fruit. Plant J. 2000 November; 24(3): 413-9.). By this means, the lycopene present in the wild-type fruit is converted to β-carotene to an increased extent.

Sridhar Dharmapuri et al. describe the overexpression of a β-cyclase and the combination with the overexpression of a β-hydroxylase in tomato fruit (Dharmapuri S, Rosati C, Pallara P, Aquilani R, Bouvier F, Camara B, Giuliano G. Metabolic engineer-ing of xanthophyll content in tomato fruits, FEBS Lett. 2002 May 22; 519(1-3):30-4). The content of α-carotenoids, here lutein, is estimated. The amounts of lutein are in all cases between 1.0 and 2.0 μg/mg of fresh weight (wild-type: 1.9 μg/mg) and the proportion of lutein of the total carotenoids is in all cases described between 1.4 and 2.9% (wild-type: 2.8%). The proportion of lutein is not significantly reduced in any fruit of these transgenic plants.

EP 393690 B1, WO 91/13078 A1, EP 735137 A1, EP 747483 A1 and WO 97/36998 A1 describe β-cyclase genes.

EP 393690 describes a process for the production of carotenoids by utilization of at least one of the genes coding for phytoene synthase, phytoene dehydrogenase, β-cyclase and β-hydroxylase obtained from Erwinia uredovora.

WO91/13078 A1 describes a process for the production of carotenoids by utilization of the genes selected from GGPP synthase, phytoene synthase, phytoene dehydrogenase, β-cyclase and β-hydroxylase obtained from Erwinia herbicola.

WO 96/36717 describes a process for the production of carotenoids by utilization of genes coding for β-cyclase obtained from Capsicum annum.

EP 747 483 A1 describes a process for the production of carotenoids by utilization of the genes coding for GGPP synthase, phytoene synthase, phytoene dehydrogenase, β-cyclase and β-hydroxylase obtained from Flavobacterium.

WO 96/28014 describes and claims DNA sequences coding for a β-cyclase from Synechococcus sp. PCC7942, tobacco and tomato.

WO 00/08920 describes a novel β-cyclase gene from tomato (Bgene), the use of the regulation signals of the Bgene for the chromoplast-specific expression of foreign genes, and the use of the antisense DNA of Bgene for the reduction of the β-carotenoid content in tomato. WO 00/08920 further describes that the Bgene can be overexpressed for the production of carotenoids in higher plants.

WO 00/32788 describes a process for the manipulation of the carotenoid content in plants by means of β-cyclase genes from marigold. WO 00/32788 further describes genetically modified marigold plants which overexpress a β-cyclase. WO 00/32788 further describes genetically modified marigold plants having a reduced ε-cyclase activity.

All processes of the prior art in some cases indeed yield a higher content of β-carotenoids in the total carotenoid content, but without significantly lowering the amount of α-carotenoids. The specific lowering of the content of α-carotenoids according to the processes of the prior art, such as, for example, the reduction of the ε-cyclase activity, leads, however, to a decrease in the total carotenoid content.

The invention is therefore based on the object of making available an alternative process for the production of β-carotenoids by culturing genetically modified plants, or making available further transgenic plants which produce β-carotenoids, which do not have the disadvantages of the prior art outlined and yield a high content of β-carotenoids, with a simultaneously smaller amount of α-carotenoids.

Accordingly, a process for the production of β-carotenoids has been found by culturing genetically modified plants which, in comparison to the wild-type, have an increased β-cyclase activity in plant tissues, comprising photosynthetically inactive plastids, and the increased β-cyclase activity is caused by a β-cyclase comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, with the proviso that tomato is excluded as a plant.

Surprisingly, it has been found that the increase in the β-cyclase activity in plant tissues comprising photosynthetically inactive plastids, caused by a β-cyclase comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, in genetically modified plants with the exception of tomato leads to an increase in the content of β-carotenoids and to a lowering of the content of α-carotenoids.

β-Cyclase activity is understood as meaning the enzyme activity of a β-cyclase.

A β-cyclase is understood as meaning a protein which has the enzymatic activity to convert a terminal, linear radical of lycopene to a β-ionone ring.

In particular, a β-cyclase is understood as meaning a protein which has the enzymatic activity to convert lycopene to γ-carotene, γ-carotene to γ-carotene or lycopene to β-carotene.

Accordingly, β-cyclase activity is understood as meaning the amount of lycopene or γ-carotene reacted or the amount of γ-carotene or β-carotene formed in a certain time by the protein β-cyclase.

In the case of an increased β-cyclase activity compared with the wild-type, the amount of lycopene or γ-carotene reacted or the amount of γ-carotene or β-carotene formed in a certain time is increased in comparison to the wild-type by the protein β-cyclase.

Preferably, this increase in the β-cyclase activity is at least 5%, further preferably at least 20%, further preferably at least 50%, further preferably at least 100%, more preferably at least 300%, even more preferably at least 500%, in particular at least 600%, of the β-cyclase activity of the wild-type.

The determination of the β-cyclase activity in genetically modified plants according to the invention and in wild-type or reference plants is preferably carried out under the following conditions:

The activity of the β-cyclase is determined in vitro according to Fraser and Sandmann (Biochem. Biophys. Res. Comm. 185(1) (1992) 9-15). Potassium phosphate as buffer (pH 7.6), lycopene as substrate, stroma protein from paprika, NADP+, NADPH and ATP are added to a specific amount of plant extract.

The in-vitro assay is carried out in a volume of 250 μl. The batch contains 50 mM potassium phosphate (pH 7.6), different amounts of plant extract, 20 nM lycopene, 250 ∝g of chromoplastidic stroma protein from paprika, 0.2 mM NADP+, 0.2 mM NADPH and 1 mM ATP. NADP/NADPH and ATP are dissolved in 10 ml of ethanol with 1 mg of Tween 80 immediately before the addition to the incubation medium. After a reaction time of 60 minutes at 30° C., the reaction is ended by addition of chloroform/methanol (2:1). The reaction products extracted in chloroform are analyzed by means of HPLC.

An alternative assay using a radioactive substrate is described in Fraser and Sandmann (Biochem. Biophys. Res. Comm. 185(1) (1992) 9-15).

The term “photosynthetically inactive plastids” is understood as meaning plastids in which no photosynthesis takes place, such as, for example, chromoplasts, leucoplasts or amyloplasts.

Accordingly, the term “plant tissue comprising photosynthetically inactive plastids” is understood as meaning plant tissue or plant parts which comprise(s) plastids in which no photosynthesis takes place, that is, for example, plant tissue or plant parts which comprise(s) chromoplasts, leucoplasts or amyloplasts, such as, for example, flowers, fruits or tubers.

In one embodiment which is described below in detail, the plant tissue comprising photosynthetically inactive plastids is selected from the group consisting of flowers, fruits and tubers.

Depending on the starting plant or corresponding genetically modified plant used, the plant in this preferred embodiment has an increased β-cyclase activity in flowers, fruits or tubers in comparison to the wild-type.

It is advantageous here for each plant to choose the plant tissue comprising photosynthetically inactive plastids, that is preferably flowers, fruit or tubers, in which the highest total carotenoid content is already present in the wild-type.

The term “wild-type” is understood according to the invention as meaning the corresponding starting plant which is not genetically modified.

Depending on the context, the term “plant” can be understood as meaning the starting plant (wild-type) or a genetically modified plant according to the invention or both.

Preferably, and in particular in cases in which the plant or the wild-type cannot be clearly assigned, “wild-type” is understood as meaning, for the increase in the β-cyclase activity, for the increase in the hydroxylase activity described below, for the reduction of the endogenous β-hydroxylase activity described below, for the reduction of the ε-cyclase activity described below and the increase in the content of β-carotenoids, in each case a reference plant.

This reference plant is, for plants which as the wild-type have the highest content of carotenoids in flowers, preferably Tagetes erecta, Tagetes patula, Tagetes lucida, Tagetes pringlei, Tagetes palmeri, Tagetes minuta or Tagetes campanulata, particularly preferably Tagetes erecta.

This reference plant is, for plants which as the wild-type have the highest content of carotenoids in fruits, preferably corn.

This reference plant is, for plants which as the wild-type have the highest content of carotenoids in tubers, preferably Solanum tuberosum.

The increase in the β-cyclase activity can take place via various pathways, for example by switching off of inhibiting regulation mechanisms at the translation and protein level or by increasing the gene expression of a nucleic acid encoding a β-cyclase compared to the wild-type, for example by induction of the β-cyclase gene by activators or strong promoters or by incorporation of nucleic acids encoding a β-cyclase into the plant.

In a preferred embodiment, the increase in the β-cyclase activity compared with the wild-type takes place by the increase in the gene expression of a nucleic acid, encoding a β-cyclase, comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, compared to the wild-type.

In a further preferred embodiment, the increase in the gene expression of a nucleic acid encoding a β-cyclase takes place by incorporation of nucleic acids which encode β-cyclases comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2 in the plant.

In the transgenic plants according to the invention, in this embodiment compared with the wild-type at least one further β-cyclase gene is thus present under the control of a promoter which guarantees the expression of the β-cyclase gene in plant tissues comprising photosynthetically inactive plastids encoding a β-cyclase comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2. In this embodiment, the genetically modified plant according to the invention accordingly has, in plant tissues comprising photosynthetically inactive plastids, at least one exogenous (=heterologous) β-cyclase, comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, or has at least two endogenous nucleic acids, encoding a β-cyclase, comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2.

To this end, in principle any β-cyclase gene according to the invention, that is any nucleic acid which encodes a β-cyclase comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, can be used.

All nucleic acids mentioned in the description can be, for example, an RNA, DNA or cDNA sequence.

With genomic β-cyclase sequences from eukaryotic sources which comprise introns, in the case where the host plant is not able or cannot be made able to express the corresponding β-cyclase, preferably already processed nucleic acid sequences, such as the corresponding cDNAs, are to be used.

Examples of nucleic acids encoding a β-cyclase comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, and the corresponding β-cyclases comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, which can be used in the process according to the invention are, for example, sequences from
tomato (Bgene; WO 00/08920; nucleic acid: SEQ ID NO: 1, protein SEQ ID NO: 2).

Further natural examples of β-cyclases and β-cyclase genes which can be used in the process according to the invention can easily be found, for example, from various organisms whose genomic sequence is known, by identity comparisons of the amino acid sequences or the corresponding retranslated nucleic acid sequences from databases containing the sequences described above and in particular containing the sequence SEQ ID NO: 2.

Further natural examples of β-cyclases and β-cyclase genes can furthermore easily be found starting from the nucleic acid sequences described above, in particular starting from the sequence SEQ ID NO: 1 from various organisms whose genomic sequence is not known, by hybridization techniques in a manner known per se.

The parameters and conditions for identity comparisons and hybridization techniques used below also apply analogously for all further nucleic acids and proteins described below, which are used in preferred embodiments of the process according to the invention or of the genetically modified plants.

The hybridization can take place under moderate (low stringency) or preferably under stringent (high stringency) conditions.

Such hybridization conditions are described, for example, in Sambrook, J., Fritsch, E. F., Maniatis, T., in: Molecular Cloning (A Laboratory Manual), 2nd edition, Cold Spring Harbor Laboratory Press, 1989, pages 9.31-9.57 or in Current Protocols in Molecular Biology, John Wiley & Sons, N.Y. (1989), 6.3.1-6.3.6.

By way of example, the conditions during the washing step can be selected from the range of conditions restricted by those having low stringency (with 2×SSC at 50° C.) and those having high stringency (with 0.2×SSC at 50° C., preferably at 65° C.) (20×SSC: 0.3 M sodium citrate, 3 M sodium chloride, pH 7.0).

Moreover, the temperature during the washing step can be raised from moderate conditions at room temperature, 22° C., up to stringent conditions at 65° C.

The two parameters, salt concentration and temperature, can be varied simultaneously, one of the two parameters can also be kept constant and only the other can be varied. During the hybridization, denaturing agents such as, for example, formamide or SDS can also be employed. In the presence of 50% formamide, the hybridization is preferably carried out at 42° C.

Some exemplary conditions for the hybridization and washing step are given below:
(1) hybridization conditions using, for example,
   (i) 4×SSC at 65° C., or
   (ii) 6×SSC at 45° C., or
   (iii) 6×SSC at 68° C., 100 mg/ml of denatured fish sperm DNA, or
   (iv) 6×SSC, 0.5% SDS, 100 mg/ml of denatured, fragmented salmon sperm DNA at 68° C., or
   (v) 6×SSC, 0.5% SDS, 100 mg/ml of denatured, fragmented salmon sperm DNA, 50% formamide at 42° C., or
   (vi) 50% formamide, 4×SSC at 42° C., or
   (vii) 50% (vol/vol) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C., or
   (viii) 2× or 4×SSC at 50° C. (moderate conditions), or
   (ix) 30 to 40% formamide, 2× or 4×SSC at 42° C. (moderate conditions).
(2) washing steps for in each case 10 minutes with, for example,
   (i) 0.015 M NaCl/0.0015 M sodium citrate/0.1% SDS at 50° C., or
   (ii) 0.1×SSC at 65° C., or
   (iii) 0.1×SSC, 0.5% SDS at 68° C., or
   (iv) 0.1×SSC, 0.5% SDS, 50% formamide at 42° C., or
   (v) 0.2×SSC, 0.1% SDS at 42° C., or
   (vi) 2×SSC at 65° C. (moderate conditions).

In a preferred embodiment of the process according to the invention, nucleic acids are incorporated which encode a protein comprising the amino acid sequence SEQ ID NO: 2 or a sequence derived from this sequence by substitution, insertion or deletion of amino acids, which has an identity of at least 65%, preferably at least 70%, more preferably at least 75%, more preferably at least 80%, more preferably at least 85%, more preferably at least 90%, more preferably at least 95%, particularly preferably at least 97%, at the amino acid level with the sequence SEQ ID NO: 2 and has the enzymatic property of a β-cyclase.

A natural β-cyclase sequence, which can be found as described above by identity comparison of the sequences or from other organisms using hybridization techniques or an artificial β-cyclase sequence can be concerned here, which, starting from the sequence SEQ ID NO: 2, has been modified by artificial variation, for example by substitution, insertion or deletion of amino acids.

The term “substitution” is to be understood in the description as meaning the replacement of one or more amino acids by one or more amino acids. Preferably, “conservative replacements” are carried out in which the amino acid replaced has a similar property to the original amino acid, for example replacement of Glu by Asp, Gln by Asn, Val by Ile, Leu by Ile, Ser by Thr.

Deletion is the replacement of an amino acid by a direct bond. Preferred positions for deletions are the termini of the polypeptide and the linkages between the individual protein domains.

Insertions are additions of amino acids to the polypeptide chain, where formally a direct bond is replaced by one or more amino acids.

Identity between two proteins is understood as meaning the identity of the amino acids over the total protein length in each case, in particular the identity which is calculated by comparison with the aid of the Lasergene software of DNASTAR, inc. Madison, Wis. (USA) using the Clustal method (Higgins D G, Sharp P M. Fast and sensitive multiple sequence alignments on a microcomputer. Comput Appl. Biosci. 1989 April; 5(2):151-1) with adjustment of the following parameters:

| Multiple alignment parameter: | |
|---|---|
| Gap penalty | 10 |
| Gap length penalty | 10 |
| Pairwise alignment parameter: | |
| K-tuple | 1 |
| Gap penalty | 3 |
| Window | 5 |
| Diagonals saved | 5 |

A protein which has an identity of at least 20% at the amino acid level with a certain sequence is accordingly understood as meaning a protein which, in a comparison of its sequence with the determined sequence, in particular according to the above program logarithm with the above parameter set, has an identity of at least 20%.

A protein which has an identity of at least 60% at the amino acid level with the sequence SEQ ID NO: 2 is accordingly understood as meaning a protein which, in a comparison of its sequence with the sequence SEQ ID NO: 2, in particular according to the above program logarithm with the above parameter set, has an identity of at least 60%.

Suitable nucleic acid sequences are obtainable, for example, by retranslation of the polypeptide sequence according to the genetic code.

Preferably, those codons are used for this which are frequently used according to the plant-specific codon usage. The codon usage can be easily determined by means of computer analyses of other, known genes of the organisms concerned.

In a particularly preferred embodiment, a nucleic acid comprising the sequence SEQ ID NO: 1 is introduced into the plant.

All abovementioned β-cyclase genes can furthermore be prepared in a manner known per se by chemical synthesis from the nucleotide structural units such as, for example, by fragment condensation of individual overlapping, complementary nucleic acid structural units of the double helix. The chemical synthesis of oligonucleotides can be carried out, for example, in a known manner, according to the phosphoamidite method (Voet, Voet, 2nd edition, Wiley Press New York, pp. 896-897). The addition of synthetic oligonucleotides and filling of gaps with the aid of the Klenow fragment of the DNA polymerase and ligation reactions, and general cloning processes are described in Sambrook et al. (1989), Molecular cloning: A laboratory manual, Cold Spring Harbor Laboratory Press.

In the process according to the invention, the expression of the β-cyclase, comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, takes place under the control of regulation signals, preferably a promoter and plastidic transit peptides, which guarantee the expression of the β-cyclase in the plant tissues comprising photo-synthetically inactive plastids.

In a preferred embodiment, genetically modified plants are used which, in plant tissues comprising photosynthetically inactive plastids, have the highest expression rate of the β-cyclase comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2.

Preferably, this is achieved by carrying out the expression of the β-cyclase according to the invention under the control of a promoter which is specific for the plant tissue.

For the case described above where the expression is to take place in flowers, it is advantageous that the expression of the β-cyclase according to the invention takes place under the control of a flower-specific or more preferably petal-specific promoter.

For the case described above where the expression is to take place in fruits, it is advantageous that the expression of the β-cyclase according to the invention takes place under the control of a fruit-specific promoter.

For the case described above where the expression is to take place in tubers, it is advantageous that the expression of the β-cyclase according to the invention takes place under the control of a tuber-specific promoter.

In a preferred embodiment, genetically modified plants are cultured which, compared to the wild-type, additionally have an increased hydroxylase activity.

Hydroxylase activity is understood as meaning the enzyme activity of a β-carotene hydroxylase, which is called a hydroxylase below.

A hydroxylase is understood as meaning a protein which has the enzymatic activity to introduce a hydroxyl group into the, optionally substituted, β-ionone ring of carotenoids.

In particular, a hydroxylase is understood as meaning a protein which has the enzymatic activity to convert β-carotene to zeaxanthin.

Accordingly, hydroxylase activity is understood as meaning the amount of β-carotene reacted in a certain time or the amount of zeaxanthin formed by the protein hydroxylase.

In the case of an increased hydroxylase activity compared with the wild-type, in comparison to the wild-type the amount of β-carotene reacted or the amount of zeaxanthin formed is thus increased in a certain time by the protein hydroxylase.

Preferably, this increase in the hydroxylase activity is at least 5%, further preferably at least 20%, further preferably at least 50%, further preferably at least 100%, more preferably at least 300%, even more preferably at least 500%, in particular at least 600%, of the hydroxylase activity of the wild-type.

The “endogenous β-hydroxylase” described below is understood as meaning the plant-intrinsic, endogenous hydroxylase. The determination of the activity is carried out analogously.

The determination of the hydroxylase activity in genetically modified plants according to the invention and in wild-type or reference plants is preferably carried out under the following conditions:

The activity of the hydroxylase is determined in vitro according to Bouvier et al. (Biochim. Biophys. Acta 1391 (1998), 320-328). It is added to a precise amount of plant extract ferredoxin, ferredoxin-NADP oxidoreductase, catalase, NADPH, and beta-carotene with mono- and digalactosyl glycerides.

Particularly preferably, the determination of the hydroxylase activity is carried out under the following conditions according to Bouvier, Keller, d'Harlingue and Camara (Xanthophyll biosynthesis: molecular and functional characterization of carotenoid hydroxylases from pepper fruits (Capsicum annuum L.; Biochim. Biophys. Acta 1391 (1998), 320-328):

The in-vitro assay is carried out in a volume of 0.250 ml. The batch contains 50 mM potassium phosphate (pH 7.6), 0.025 mg of ferredoxin from spinach, 0.5 units of ferredoxin-NADP+oxidoreductase from spinach, 0.25 mM NADPH, 0.010 mg of beta-carotene (emulsified in 0.1 mg of Tween 80), 0.05 mM of a mixture of mono- and digalactosyl glycerides (1:1), 1 unit of catalase (1:1), 0.2 mg of bovine serum albumin and plant extract in a differing volume. The reaction mixture is incubated at 30° C. for 2 hours. The reaction products are extracted with organic solvents such as acetone or chloroform/methanol (2:1) and determined by means of HPLC.

The increase in the hydroxylase activity can take place by various pathways, for example by switching-off of inhibiting regulation mechanisms at the expression and protein level or by increase in the gene expression of nucleic acids encoding a hydroxylase compared to the wild-type.

The increase in the gene expression of the nucleic acids encoding a hydroxylase compared to the wild-type can likewise take place by various pathways, for example by induction of the hydroxylase gene by activators or by introduction of one or more hydroxylase gene copies, that is by introduction of at least one nucleic acid encoding a hydroxylase into the plant.

Increase in the gene expression of a nucleic acid encoding a hydroxylase is understood according to the invention also as meaning the manipulation of the expression of the plant-intrinsic, endogenous hydroxylase.

This can be achieved, for example, by modification of the promoter DNA sequence for hydroxylase-encoding genes. Such a modification, which can result in an increased expression rate of the gene, can be carried out, for example, by deletion or insertion of DNA sequences.

It is, as described above, possible to modify the expression of the endogenous hydroxylase by the application of exogenous stimuli. This can be carried out by means of special physiological conditions, that is by the application of foreign substances.

In addition, modified or increased expression of an endogenous hydroxylase gene can be achieved by a regulator protein not occurring in the plant which is not transformed interacting with the promoter of this gene.

Such a regulator can be a chimeric protein, which consists of a DNA binding domain and a transcription activator domain, such as described, for example, in WO 96/06166.

In specific preferred plants, in which the main focus of the biosynthesis is on the α-carotenoid pathway, such as, for example, plants of the genus Tagetes, it is advantageous to reduce the endogenous β-hydroxylase activity and to increase the activity of exogenous hydroxylases.

In a preferred embodiment, the increase in the gene expression of a nucleic acid encoding a hydroxylase takes place by introduction of at least one nucleic acid encoding a hydroxylase into the plant.

To this end, in principle any hydroxylase gene, that is any nucleic acid which encodes a hydroxylase, can be used.

In the case of genomic hydroxylase sequences from eukaryotic sources which comprise introns, for the case where the host plant i's not able or cannot be made able to express the corresponding hydroxylase, already processed nucleic acid sequences, such as the corresponding cDNAs, are preferably to be used.

Examples of hydroxylase genes are:
a nucleic acid, encoding a hydroxylase from Haematococcus pluvialis, Accession No. AX038729, WO 0061764); (nucleic acid: SEQ ID NO: 3, protein: SEQ ID NO: 4),
and hydroxylases of the following accession numbers:
   |emb|CAB55626.1, CM70427.1, CAA70888.1, CAB55625.1, AF499108_{—}1, AF315289_{—}1, AF296158_{—}1, AAC49443.1, NP_{—}194300.1, NP_{—}200070.1, AAG10430.1, CAC06712.1, AAM88619.1, CAC95130.1, AAL80006.1, AF162276_{—}1, M053295.1, AAN85601.1, CRTZ_ERWHE, CRTZ_PANAN, BAB79605.1, CRTZ_ALCSP, CRTZ_AGRAU, CAB56060.1, ZP_{—}00094836.1, MC44852.1, BAC77670.1, NP_{—}745389.1, NP_{—}344225.1, NP_{—}849490.1, ZP_{—}00087019.1, NP_{—}503072.1, NP_{—}852012.1, NP_{—}115929.1, ZP_{—}00013255.1

A particularly preferred hydroxylase is furthermore the hydroxylase from tomato (Acc. No. LEY14810) (nucleic acid: SEQ ID NO: 5; protein: SEQ ID NO. 6).

In the preferred transgenic plants according to the invention, at least one further hydroxylase gene is present in this preferred embodiment compared with the wild-type.

In this preferred embodiment, the genetically modified plant has, for example, at least one exogenous nucleic acid encoding a hydroxylase, or at least two endogenous nucleic acids encoding a hydroxylase.

Preferably, in the preferred embodiment described above, as hydroxylase genes, nucleic acids which encode proteins comprising the amino acid sequence SEQ ID NO: 6 or a sequence derived from this sequence by substitution, insertion or deletion of amino acids, which have an identity of at least 20%, preferably of at least 50%, more preferably of at least 70%, even more preferably of at least 90%, most preferably of at least 95% at the amino acid level with the sequence SEQ ID NO: 6, and which have the enzymatic property of a hydroxylase, are used.

Further examples of hydroxylases and hydroxylase genes can easily be found, for example, from various organisms whose genomic sequence is known, as described above, by homology comparisons of the amino acid sequences or the corresponding retranslated nucleic acid sequences from databases with the sequence SEQ ID NO: 6.

Further examples of hydroxylases and hydroxylase genes can furthermore, for example, easily be found starting from the sequence SEQ ID NO: 5 of various organisms whose genomic sequence is not known, as described above, by hybridization and PCR techniques in a manner known per se.

In a further particularly preferred embodiment, for increasing the hydroxylase activity, nucleic acids are introduced into organisms which encode proteins comprising the amino acid sequence of the hydroxylase of the sequence SEQ ID NO: 6.

Suitable nucleic acid sequences are obtainable, for example, by retranslation of the polypeptide sequence according to the genetic code.

Preferably, for this those codons are used which, corresponding to the plant-specific codon usage, are frequently used. The codon usage can easily be determined by means of computer analyses of other, known genes of the organisms concerned.

In a particularly preferred embodiment, a nucleic acid comprising the sequence SEQ ID NO: 5 is introduced into the organism.

All abovementioned hydroxylase genes can furthermore be prepared in a manner known per se by chemical synthesis from the nucleotide structural units such as, for example, by fragment condensation of individual overlapping, complementary nucleic acid structural units of the double helix. The chemical synthesis of oligonucleotides can be carried out, for example, in a known manner, according to the phosphoamidite method (Voet, Voet, 2nd edition, Wiley Press New York, page 896-897). The addition of synthetic oligonucleotides and filling of gaps with the aid of the Klenow fragment of the DNA polymerase and ligation reactions, and general cloning processes are described in Sambrook et al. (1989), Molecular cloning: A laboratory manual, Cold Spring Harbor Laboratory Press.

Preferably, the expression of the hydroxylase in the process according to the invention takes place under the control of regulation signals, preferably a promoter and plastid transit peptides which guarantee the expression of the hydroxylase in the plant tissues comprising photosynthetically inactive plastids.

In a particularly preferred embodiment, genetically modified plants are used which have the highest expression rate of the hydroxylase in plant tissues comprising photosynthetically inactive plastids.

Preferably, this is achieved by the expression of the hydroxylase taking place under the control of a promoter specific for the plant tissue.

For the case described above, where the expression is to take place in flowers, it is advantageous that the additional expression of the hydroxylase takes place under the control of a flower-specific or preferably petal-specific promoter.

For the case described above, where the expression is to take place in fruits, it is advantageous that the additional expression of the hydroxylase takes place under the control of a fruit-specific promoter.

For the case described above, where the expression is to take place in tubers, it is advantageous that the additional expression of the hydroxylase takes place under the control of a tuber-specific promoter.

In a further preferred embodiment of the process, the genetically modified plants, compared with the wild-type, additionally have a reduced activity of at least one of the activities selected from the group consisting of ε-cyclase activity and endogenous β-hydroxylase activity.

ε-Cyclase activity is understood as meaning the enzyme activity of an ε-cyclase.

An ε-cyclase is understood as meaning a protein which has the enzymatic activity to convert a terminal, linear residue of lycopene to an ε-ionone ring.

An ε-cyclase is therefore in particular understood as meaning a protein which has the enzymatic activity to convert lycopene to δ-carotene.

Accordingly, ε-cyclase activity is understood as meaning the amount of lycopene reacted or the amount of δ-carotene formed in a certain time by the protein ε-cyclase.

In the case of a reduced ε-cyclase to activity compared with the wild-type, in comparison to the wild-type, the amount of lycopene reacted or the amount of δ-carotene formed in a certain time by the protein ε-cyclase is thus reduced.

The determination of the ε-cyclase activity in genetically modified plants according to the invention and in wild-type or reference plants is preferably carried out under the following conditions:

The ε-cyclase activity can be determined in vitro according to Fraser and Sandmann (Biochem. Biophys. Res. Comm. 185(1) (1992) 9-15), if potassium phosphate as buffer (pH 7.6), lycopene as substrate, stroma protein from paprika, NADP+, NADPH and ATP are added to a defined amount of plant extract.

The determination of the ε-cyclase activity in genetically modified plants according to the invention and in wild-type or reference plants is particularly preferably carried out according to Bouvier, d'Harlingue and Camara (Molecular Analysis of carotenoid cyclase inhibition; Arch. Biochem. Biophys. 346(1) (1997) 53-64):

The in-vitro assay is carried out in a volume of 0.25 ml. The batch contains 50 mM of potassium phosphate (pH 7.6), different amounts of plant extract, 20 nM lycopene, 0.25 mg of chromoplastidic stroma protein from paprika, 0.2 mM NADP+, 0.2 mM NADPH and 1 mM ATP. NADP/NADPH and ATP are dissolved in 0.01 ml of ethanol with 1 mg of Tween 80 immediately before the addition to the incubation medium. After a reaction time of 60 minutes at 30° C., the reaction is ended by addition of chloroform/methanol (2:1). The reaction products extracted in chloroform are analyzed by means of HPLC.

An alternative assay using radioactive substrate is described in Fraser and Sandmann (Biochem. Biophys. Res. Comm. 185(1) (1992) 9-15). A further analytical method is described in Beyer, Kröncke and Nievelstein (On the mechanism of the lycopene isomerase/cyclase reaction in Narcissus pseudonarcissus L. chromoplast, J. Biol. Chem. 266(26) (1991) 17072-17078).

Endogenous β-hydroxylase activity is understood as meaning the enzyme activity of the endogenous, plant-intrinsic β-hydroxylase.

An endogenous β-hydroxylase is understood as meaning an endogenous, plant-intrinsic hydroxylase as described above. If, for example, Tagetes erecta is the target plant to be genetically modified, the endogenous β-hydroxylase is to be understood as meaning the β-hydroxylase of Tagetes erecta.

An endogenous β-hydroxylase is accordingly understood in particular as meaning a plant-intrinsic protein, which has the enzymatic activity to convert β-carotene to zeaxanthin.

Accordingly, endogenous β-hydroxylase activity is understood as meaning the amount of α-carotene reacted or the amount of zeaxanthin formed in a certain time by the protein endogenous β-hydroxylase.

In the case of a reduced endogenous β-hydroxylase activity compared with the wild-type, in comparison to the wild-type, the amount of β-carotene reacted or the amount of zeaxanthin formed in a certain time by the protein endogenous β-hydroxylase is thus reduced.

Preferably, this reduction of the endogenous β-hydroxylase activity is at least 5%, further preferably at least 20%, further preferably at least 50%, further preferably 100%.

Particularly preferably, the endogenous β-hydroxylase activity is completely switched off.

It has surprisingly been found that in the case of plants which in the majority produce carotenoids of the α-carotene pathway, such as, for example, lutein, such as, for example, plants of the genus Tagetes, it is advantageous to reduce the activity of the endogenous β-hydroxylase and optionally to increase the activity of a heterologous hydroxylase. Particularly preferably, hydroxylases or functional equivalents thereof are used here which originate from plants which in the majority produce carotenoids of the β-carotene pathway, such as, for example, the β-hydroxylase from tomato described above (nucleic acid: SEQ ID No. 5, protein: SEQ ID No. 6).

The determination of the endogenous β-hydroxylase activity is carried out as described above analogously to the determination of the hydroxylase activity.

A reduced ε-cyclase activity or hydroxylase activity is preferably the partial or essentially complete suppression or blocking, based on different cytobiological mechanisms, of the functionality of an ε-cyclase or hydroxylase in a plant cell, plant or a part derived therefrom, tissue, organ, cells or seeds.

The reduction of the enzyme activities according to the invention in plants compared to the wild-type can take place, for example, by reduction of the amount of protein, or the amount of mRNA in the plant. Accordingly, a reduced enzyme activity compared with the wild-type can be determined directly or can take place via the determination of the amount of protein or the amount of mRNA of the plants according to the invention in comparison to the wild-type.

A reduction of the ε-cyclase activity comprises a quantitative decrease in an ε-cyclase down to an essentially complete absence of the ε-cyclase (i.e. lacking detectability of ε-cyclase activity or lacking immunological detectability of the ε-cyclase). Preferably, the ε-cyclase activity (or the amount of ε-cyclase protein or the amount of ε-cyclase mRNA) in the plants, particularly preferably in flowers in comparison to the wild-type is reduced by at least 5%, further preferably by at least 20%, further preferably by at least 50%, further preferably by 100%. In particular, “reduction” also means the complete absence of the ε-cyclase activity (or of the ε-cyclase protein or of the ε-cyclase mRNA).

A reduction in the endogenous β-hydroxylase activity comprises a quantitative decrease in an endogenous β-hydroxylase down to an essentially complete absence of the endogenous β-hydroxylase (i.e. lacking detectability of endogenous β-hydroxylase activity or lacking immunological detectability of the endogenous β-hydroxylase). Preferably, the endogenous β-hydroxylase activity (or the amount of endogenous β-hydroxylase protein or the amount of endogenous β-hydroxylase mRNA) in the plant, particularly preferably in flowers in comparison to the wild-type, is reduced by at least 5%, further preferably by at least 20%, further preferably by at least 50%, further preferably by 100%. In particular, “reduction” also means the complete absence of the endogenous β-hydroxylase activity (or of the endogenous β-hydroxylase protein or of the endogenous β-hydroxylase mRNA).

Preferably, the reduction of the ε-cyclase activity and/or the endogenous β-hydroxylase activity in plants takes place by at least one of the following processes:
a) introduction of at least one double-stranded ε-cyclase ribonucleic acid sequence and/or endogenous β-hydroxylase ribonucleic acid sequence or an expression cassette or expression cassettes guaranteeing their expression in plants. Those processes are included in which the dsRNA is directed against a gene (that is DNA sequences such as the promoter sequence) or a transcript (that is mRNA sequences),
b) introduction of at least one ε-cyclase antisense ribonucleic acid sequence and/or endogenous β-hydroxylase antisense ribonucleic acid sequence or an expression cassette or expression cassettes guaranteeing their expression in plants. Those processes are included in which the antisense RNA is directed against a gene (that is genomic DNA sequences) or a gene transcript (that is RNA sequences). α-Anomeric nucleic acid sequences are included,
c) introduction of at least one ε-cyclase antisense ribonucleic acid sequence and/or endogenous β-hydroxylase antisense ribonucleic acid sequence in each case combined with a ribozyme or an expression cassette or expression cassettes guaranteeing their expression in plants,
d) introduction of at least one ε-cyclase sense ribonucleic acid sequence and/or endogenous-β-hydroxylase sense ribonucleic acid sequence for the induction of a cosuppression or an expression cassette or expression cassettes guaranteeing their expression in plants,
e) introduction of at least one DNA- or protein-binding factor against an ε-cyclase gene, RNA or protein and/or endogenous β-hydroxylase gene, RNA or protein or an expression cassette or expression cassettes guaranteeing its expression in plants,
f) introduction of at least one viral nucleic acid sequence or nucleic acid sequences bringing about the ε-cyclase-RNA and/or endogenous β-hydroxylase RNA degradation or an expression cassette or expression cassettes guaranteeing their expression in plants,
g) introduction of at least one construct for the production of an insertion, deletion, inversion or mutation in an ε-cyclase gene and/or endogenous β-hydroxylase gene in plants. The method comprises the introduction of at least one construct for the production of a loss of function, such as, for example, the generation of stop codons or a shift in the reading frame, onto a gene, for example by production of an insertion, deletion, inversion or mutation in a gene. Preferably, knockout mutants can be generated by means of specific insertion in said gene by homologous recombination or introduction of sequence-specific nucleases against the corresponding gene sequences.

It is known to the person skilled in the art that further processes can also be employed in the context of the present invention for the decrease in an ε-cyclase and/or endogenous β-hydroxylase or its activity or function. For example, the introduction of a dominant-negative variant of an ε-cyclase or endogenous β-hydroxylase or an expression cassette guaranteeing their expression can also be advantageous. Here, any individual one of these processes can bring about a decrease in the amount of protein, amount of mRNA and/or activity of an ε-cyclase or endogenous β-hydroxylase. Combined use is also conceivable. Further methods are known to the person skilled in the art and can include the hindrance or suppression of the processing of the ε-cyclase or endogenous β-hydroxylase, the transport of the ε-cyclase or endogenous β-hydroxylase or their mRNAs, inhibition of the ribosome addition, inhibition of RNA splicing, induction of an enzyme degrading ε-cyclase RNA or endogenous β-hydroxylase RNA and/or inhibition of translational elongation or termination.

The individual preferred processes may as a result be described by exemplary embodiments:
a) introduction of a double-stranded ε-cyclase ribonucleic acid sequence (ε-cyclase dsRNA) or double-stranded endogenous β-hydroxylase ribonucleic acid sequence (endogenous β-hydroxylase dsRNA)

The process of gene regulation by means of double-stranded RNA (“double-stranded RNA interference”; dsRNAi) is known and described, for example, in Matzke M A et al. (2000) Plant Mol Biol 43:401-415; Fire A. et al (1998) Nature 391:806-811; WO 99/32619; WO 99/53050; WO 00/68374; WO 00/44914; WO 00/44895; WO 00/49035 or WO 00/63364. Reference is hereby expressly made to the processes and methods described in the citations indicated.

“Double-stranded ribonucleic acid sequence” is understood according to the invention as meaning one or more ribonucleic acid sequences, which on the basis of complementary sequences in theory, for example according to the base pair rules of Watson and Crick and/or in reality, for example on the basis of hybridization experiments, are able in vitro and/or in vivo to form double-stranded RNA structures.

The person skilled in the art is aware that the formation of double-stranded RNA structures represents an equilibrium state. Preferably, the ratio of double-stranded molecules to corresponding dissociated forms is at least 1 to 10, preferably 1:1, particularly preferably 5:1, most preferably 10:1.

A double-stranded ε-cyclase ribonucleic acid sequence or alternatively ε-cyclase dsRNA is preferably understood as meaning an RNA molecule which has a region with double-strand structure and in this region comprises a nucleic acid sequence which
a) is identical with at least one part of the plant-intrinsic ε-cyclase transcript and/or
b) is identical with at least one part of the plant-intrinsic ε-cyclase promoter sequence.

In the process according to the invention, for the reduction of the ε-cyclase activity an RNA is therefore preferably introduced into the plant which has a region with double-strand structure and in this region comprises a nucleic acid sequence which
a) is identical with at least one part of the plant-intrinsic ε-cyclase transcript and/or
b) is identical with at least one part of the plant-intrinsic ε-cyclase promoter sequence.

The term “ε-cyclase transcript” is understood as meaning the transcribed part of an ε-cyclase gene, which in addition to the ε-cyclase encoding sequence, for example, also comprises nonencoding sequences, such as, for example, also UTRs.

By an RNA, which “is identical with at least one part of the plant-intrinsic ε-cyclase promoter sequence”, it is preferably meant that the RNA sequence is identical with at least one part of the theoretical transcript of the ε-cyclase promoter sequence, that is the corresponding RNA sequence.

“One part” of the plant-intrinsic ε-cyclase transcript or of the plant-intrinsic ε-cyclase-promoter sequence is understood as meaning subsequences which can extend from a few base pairs as far as complete sequences of the transcript or of the promoter sequence. The person skilled in the art can easily determine the optimum length of the subsequences by routine experiments.

A double-stranded endogenous β-hydroxylase ribonucleic acid sequence or alternatively endogenous β-hydroxylase dsRNA is preferably understood as meaning an RNA molecule which has a region having double-strand structure and in this region comprises a nucleic acid sequence which
a) is identical with at least one part of the plant-intrinsic, endogenous β-hydroxylase transcript and/or
b) is identical with at least one part of the plant-intrinsic, endogenous β-hydroxylase promoter sequence.

In the process according to the invention, for the reduction of the endogenous β-hydroxylase activity an RNA is therefore preferably introduced into the plant which has a region having double-strand structure and in this region comprises a nucleic acid sequence which
a) is identical with at least one part of the plant-intrinsic, endogenous β-hydroxylase transcript and/or
b) is identical with at least one part of the plant-intrinsic, endogenous β-hydroxylase promoter sequence.

The term “endogenous β-hydroxylase transcript” is understood as meaning the transcribed part of an endogenous β-hydroxylase gene, which in addition to the endogenous β-hydroxylase encoding sequence, for example, also comprises nonencoding sequences, such as, for example, also UTRs.

By an RNA which “is identical with at least one part of the plant-intrinsic, endogenous β-hydroxylase promoter sequence”, it is preferably meant that the RNA sequence is identical with at least one part of the theoretical transcript of the endogenous β-hydroxylase promoter sequence, that is of the corresponding RNA sequence.

“One part” of the plant-intrinsic, endogenous β-hydroxylase transcript or of the plant-intrinsic endogenous β-hydroxylase promoter sequence is understood as meaning subsequences which can extend from a few base pairs as far as complete sequences of the transcript or of the promoter sequence. The person skilled in the art can easily determine the optimum length of the subsequences by routine experiments.

As a rule, the length of the subsequences is at least 10 bases and at most 2 kb, preferably at least 25 bases and at most 1.5 kb, particularly preferably at least 50 bases and at most 600 bases, very particularly preferably at least 100 bases and at most 500, most preferably at least 200 bases or at least 300 bases and at most 400 bases.

Preferably, the subsequences are chosen such that a specificity as high as possible is achieved and activities of other enzymes are not reduced whose decrease is not desired. It is therefore advantageous to choose for the subsequences of the dsRNA parts of the transcripts and/or subsequences of the promoter sequences which do not occur in other activities.

In a particularly preferred embodiment, the dsRNA therefore comprises a sequence which is identical with one part of the plant-intrinsic ε-cyclase transcript or endogenous β-hydroxylase transcript and comprises the 5′ end or the 3′ end of the plant-intrinsic nucleic acid, encoding an ε-cyclase or endogenous β-hydroxylase. In particular, untranslated regions in 5′ or 3′ of the transcript are suitable for preparing selective double-strand structures.

A further subject of the invention relates to double-stranded RNA molecules (dsRNA molecules), which on introduction into a plant organism (or a cell, tissue, organ or proliferation material derived therefrom) bring about the decrease in an ε-cyclase or an endogenous β-hydroxylase.

A double-stranded RNA molecule for the reduction of the expression of an ε-cyclase (ε-cyclase dsRNA) here preferably comprises
a) a “sense” RNA strand comprising at least one ribonucleotide sequence, which is essentially identical to at least one part of a “sense” RNA ε-cyclase transcript, and
b) an “antisense” RNA strand, which is essentially, preferably completely, complementary to the RNA “sense” strand under a).

For the transformation of the plant with an endogenous β-hydroxylase dsRNA, a nucleic acid construct is preferably used which is introduced into the plant and which is transcribed in the plant into the endogenous β-hydroxylase dsRNA.

A double-stranded RNA molecule for the reduction of the expression of an endogenous β-hydroxylase (endogenous β-hydroxylase dsRNA) here preferably comprises
a) a “sense” RNA strand comprising at least one ribonucleotide sequence, which is essentially identical to at least one part of a “sense” RNA endogenous β-hydroxylase transcript, and
b) an “antisense” RNA strand, which is essentially, preferably completely, complementary to the RNA “sense” strand under a).

For the transformation of the plant with an endogenous β-hydroxylase dsRNA, a nucleic acid construct is preferably used which is introduced into the plant and which is transcribed in the plant into the endogenous β-hydroxylase dsRNA.

These nucleic acid constructs are also called expression cassettes or expression vectors below.

With respect to the dsRNA molecules, ε-cyclase nucleic acid sequence, or the corresponding transcript for the preferred plant Tagetes erecta, is preferably understood as meaning the sequence as set forth in SEQ ID NO: 8 or a part thereof.

With respect to the dsRNA molecules, endogenous β-hydroxylase nucleic acid sequence, or the corresponding transcript for the preferred plant Tagetes erecta is preferably understood as meaning the sequence as set forth in SEQ ID NO: 16 or a part thereof.

“Essentially identical” means that the dsRNA sequence can also have insertions, deletions and individual point mutations in comparison to the target sequence and nevertheless brings about an efficient decrease in the expression. Preferably, the homology is at least 75%, even more preferably at least 80%, very particularly preferably at least 90%, most preferably 100%, between the “sense” strand of an inhibitory dsRNA and at least one part of the “sense” RNA transcript, or between the “antisense” strand and the complementary strand of the corresponding gene.

A 100% strength sequence identity between dsRNA and a gene transcript is not absolutely necessary in order to bring about an efficient decrease in the protein expression. Consequently, there is the advantage that the process is tolerant to sequence differences, such as can be present as a result of genetic mutations, polymorphisms or evolutionary divergences. Thus, it is, for example, possible with the dsRNA, which was generated from the ε-cyclase sequence or endogenous β-hydroxylase sequence of the one organism, to suppress the ε-cyclase expression or endogenous β-hydroxylase expression in another organism. For this purpose, the dsRNA preferably comprises sequence regions of gene transcripts which correspond to conserved regions. Said conserved regions can easily be derived from sequence comparisons.

“Essentially complementary” means that the “antisense” RNA strand can also contain insertions, deletions and individual point mutations in comparison to the complement of the “sense” RNA strand. Preferably, the homology is at least 80%, even more preferably at least 90%, very particularly preferably at least 95%, most preferably 100%, between the “antisense” RNA strand and the complement of the “sense” RNA strand.

In a further embodiment the ε-cyclase dsRNA comprises
a) a “sense” RNA strand comprising at least one ribonucleotide sequence which is essentially identical to at least one part of the promoter sequence of an ε-cyclase gene, and
b) an “antisense” RNA strand, which is essentially—preferably completely—complementary to the RNA “sense” strand under a).

Preferably, the promoter region of an ε-cyclase for the preferred plant Tagetes erecta is understood as meaning a sequence as set forth in SEQ ID NO: 9 or a part thereof.

For the preparation of the ε-cyclase dsRNA sequences for the reduction of the ε-cyclase activity, in particular for the preferred plant Tagetes erecta, particularly preferably the following subsequences are used:
SEQ ID NO: 10: sense fragment of the 5′ terminal region of the ε-cyclase
SEQ ID NO: 11: antisense fragment of the 5′ terminal region of the ε-cyclase
SEQ ID NO: 12: sense fragment of the 3′ terminal region of the ε-cyclase
SEQ ID NO: 13: antisense fragment of the 3′ terminal region of the ε-cyclase
SEQ ID NO: 14: sense fragment of the ε-cyclase promoter
SEQ ID NO: 15: antisense fragment of the ε-cyclase promoter

In a further embodiment the endogenous β-hydroxylase dsRNA comprises
a) a “sense” RNA strand comprising at least one ribonucleotide sequence which is essentially identical to at least one part of the promoter sequence of an endogenous β-hydroxylase gene, and
b) an “antisense” RNA strand which is essentially—preferably completely—complementary to the RNA “sense” strand under a).

For the preparation of the endogenous β-hydroxylase dsRNA sequences for the reduction of the endogenous β-hydroxylase activity, in particular for the preferred plant Tagetes erecta, the following subsequences are particularly preferably used:
SEQ ID NO: 18: sense fragment of the 5′ terminal region of the endogenous β-hydroxylase
SEQ ID NO: 19: antisense fragment of the 5′ terminal region of the endogenous β-hydroxylase

The dsRNA can consist of one or more strands of polyribonucleotides. Of course, it is possible in order to achieve the same object also to introduce a number of individual dsRNA molecules, which in each case comprise one of the ribonucleotide sequence sections defined above, in the cell or the organism.

The double-stranded dsRNA structure can be formed starting from two complementary, separate RNA strands or—preferably—starting from an individual, self-complementary RNA strand. In this case, “sense” RNA strand and “antisense” RNA strand are preferably connected to one another covalently in the form of an inverted “repeat”.

As described, for example, in WO 99/53050, the dsRNA can also comprise a hairpin structure by connecting a “sense” and “antisense” strand by a connecting sequence (“linker”; for example an intron). The self-complementary dsRNA structures are preferred, since they only necessitate the expression of an RNA sequence and always comprise the complementary RNA strands in an equimolar ratio. Preferably, the connecting sequence is an intron (e.g. an intron of the ST-LS1 gene from potato; Vancanneyt G F et al. (1990) Mol Gen Genet 220(2):245-250).

The nucleic acid sequence coding for a dsRNA can contain further elements such as, for example, transcription termination signals or polyadenylation signals.

If the dsRNA, however, is directed against the promoter sequence of an enzyme, it preferably comprises no transcription termination signals or polyadenylation signals. This makes possible a retention of the dsRNA in the nucleus of the cell and prevents a distribution of the dsRNA in the entire plant (“spreading”).

If the two strands of the dsRNA in a cell or plant are to be brought together, this can occur, by way of example, in the following manner:
a) transformation of the cell or plant with a vector which comprises both expression cassettes,
b) cotransformation of the cell or plant with two vectors, one comprising the expression cassettes containing the “sense” strand, the other comprising the expression cassettes containing the “antisense” strand,
c) crossing of two individual plant lines, one comprising the expression cassettes containing the “sense” strand, the other comprising the expression cassettes containing the “antisense” strand.

The formation of the RNA duplex can be initiated either outside the cell or within the same.

The dsRNA can be synthesized either in vivo or in vitro. To this end, a DNA sequence coding for a dsRNA can be brought into an expression cassette under control of at least one genetic control element (such as, for example, a promoter). A poly-adenylation is not necessary, likewise no elements for the initiation of a translation have to be present. Preferably, the expression cassette for the MP dsRNA is present on the transformation construct or the transformation vector.

The expression cassettes coding for the “antisense” and/or the “sense” strand of an ε-cyclase dsRNA or for the self-complementary strand of the dsRNA, are for this purpose preferably inserted into a transformation vector and introduced into the plant cell using the processes described below. For the process according to the invention, a stable insertion into the genome is advantageous.

The dsRNA can be introduced in an amount which makes possible at least one copy per cell. Higher amounts (e.g. at least 5, 10, 100, 500 or 1000 copies per cell) can optionally bring about a more efficient decrease.
b) introduction of an antisense ribonucleic acid sequence of an ε-cyclase (ε-cyclase antisense RNA) or introduction of an antisense ribonucleic acid sequence of an endogenous β-hydroxylase (endogenous β-hydroxylase antisense RNA)

Processes for the decrease in a certain protein by means of the “antisense” technology have been described frequently—even in plants—(Sheehy et al. (1988) Proc Natl Acad Sci USA 85: 8805-8809; U.S. Pat. No. 4,801,340; Mol J N et al. (1990) FEBS Lett 268(2):427-430).

The antisense nucleic acid molecule hybridizes or binds with the cellular mRNA and/or genomic DNA coding for the ε-cyclase or endogenous β-hydroxylase to be decreased. By this means, the transcription and/or translation of the ε-cyclase or endogenous β-hydroxylase is suppressed.

The hybridization can result in a conventional manner via the formation of a stable duplex or—in the case of genomic DNA—by binding of the antisense nucleic acid molecule with the duplex of the genomic DNA by means of specific interaction in the major groove of the DNA helix.

An ε-cyclase antisense RNA can be derived using the nucleic acid sequence coding for this ε-cyclase, for example the nucleic acid sequence as set forth in SEQ ID NO: 7 according to the base pair rules of Watson and Crick. The ε-cyclase antisense RNA can be complementary to the entire transcribed mRNA of the ε-cyclase, be restricted to the coding region or consist only of an oligonucleotide which is complementary to a part of the coding or noncoding sequence of the mRNA. Thus the oligonucleotide, for example, can be complementary to the region which comprises the translation start for the ε-cyclase.

An endogenous β-hydroxylase antisense RNA can be derived using the nucleic acid sequence coding for this endogenous β-hydroxylase, for example the nucleic acid sequence set forth in SEQ ID NO: 16 according to the base pair rules of Watson and Crick. The endogenous β-hydroxylase antisense RNA can be complementary to the entire transcribed mRNA of the endogenous β-hydroxylase, be restricted to the coding region or consist only of an oligonucleotide which is complementary to a part of the coding or noncoding sequence of the mRNA. Thus the oligonucleotide, for example, can be complementary to the region which comprises the translation start for the endogenous β-hydroxylase.

The antisense RNAs can have a length of, for example, 5, 10, 15, 20, 25, 30, 35, 40, 45 or 50 nucleotides, but can also be longer and comprise at least 100, 200, 500, 1000, 2000 or 5000 nucleotides. The antisense RNAs are preferably expressed recombinantly in the target cell in the context of the process according to the invention.

A further subject of the invention relates to transgenic expression cassettes comprising a nucleic acid sequence coding for at least one part of an ε-cyclase or endogenous β-hydroxylase, said nucleic acid sequence being functionally linked to a promoter functional in plant organisms in antisense orientation.

Said expression cassettes can be part of a transformation construct or transformation vector, or else be introduced in the context of a cotransformation.

In a further preferred embodiment, the expression of an ε-cyclase or endogenous β-hydroxylase can be inhibited by nucleotide sequences which are complementary to the regulatory region of an ε-cyclase gene or endogenous β-hydroxylase gene (e.g. promoter and/or enhancer) and form triple-helical structures with the DNA double helix there, such that the transcription of the ε-cyclase gene or endogenous β-hydroxylase gene is reduced. Appropriate processes have been described (Helene C (1991) Anticancer Drug Res 6(6):569-84; Helene C et al. (1992) Ann NY Acad Sci 660:27-36; Maher L J (1992) Bioassays 14(12):807-815).

In a further embodiment, the antisense RNA can be an α-anomeric nucleic acid. α-Anomeric nucleic acid molecules of this type form specific double-stranded hybrids with complementary RNA in which—in contrast to the conventional β-nucleic acids—the two strands run parallel to one another (Gautier C et al. (1987) Nucleic Acids Res 15:6625-6641).
c) introduction of an ε-cyclase antisense RNA or endogenous β-hydroxylase antisense RNA combined with a ribozyme

Advantageously, the antisense strategy described above can be coupled with a ribozyme process. Catalytic RNA molecules or ribozymes can be adapted to any desired target RNA and cleave the phosphodiester structure at specific positions, whereby the target RNA is functionally deactivated (Tanner NK (1999) FEMS Microbiol Rev 23(3):257-275). The ribozyme is not itself modified thereby, but is able to cleave further target RNA molecules analogously, whereby it obtains the properties of an enzyme. The incorporation of ribozyme sequences in “antisense” RNAs imparts precisely to these “antisense” RNAs this enzymatic, RNA-cleaving property and thus increases their efficiency in the inactivation of the target RNA. The preparation and use of appropriate ribozyme “antisense” RNA molecules has been described (inter alia in Haselhoff et al. (1988) Nature 334: 585-591); Haselhoff and Gerlach (1988) Nature 334:585-591; Steinecke P et al. (1992) EMBO J 11(4):1525-1530; de Feyter Ret al. (1996) Mol Gen Genet. 250(3):329-338).

In this manner, ribozymes (e.g. “hammerhead” ribozymes; Haselhoff and Gerlach (1988) Nature 334:585-591) can be used in order to catalytically cleave the mRNA of an ε-cyclase to be decreased and thus to prevent translation. Ribozyme technology can increase the efficiency of an antisense strategy. Processes for the expression of ribozymes for the reduction of certain proteins have been described (EP 0 291 533, EP 0 321 201, EP 0 360 257). In plant cells, ribozyme expression has likewise been described (Steinecke P et al. (1992) EMBO J 11 (4):1525-1530; de Feyter R et al. (1996) Mol Gen Genet. 250(3):329-338). Suitable target sequences and ribozymes can be determined, for example, as described in “Steinecke P, Ribozymes, Methods in Cell Biology 50, Galbraith et al. eds, Academic Press, Inc. (1995), pp. 449460”, by secondary structure calculations of ribozyme and target RNA, and by their interaction (Bayley C C et al. (1992) Plant Mol. Biol. 18(2):353-361; Lloyd A M and Davis R W et al. (1994) Mol Gen Genet. 242(6):653-657). For example, derivatives of the Tetrahymena L-19 IVS RNA can be constructed which have regions complementary to the mRNA of the ε-cyclase to be suppressed (see also U.S. Pat. No. 4,987,071 and U.S. Pat. No. 5,116,742). Alternatively, such ribozymes can also be identified from a library of various ribozymes by means of a selection process (Bartel D and Szostak J W (1993) Science 261:1411-1418).
d) introduction of a sense ribonucleic acid sequence of an ε-cyclase or endogenous β-hydroxylase (ε-cyclase sense RNA or endogenous β-hydroxylase sense RNA) for the induction of cosuppression

The expression of an ε-cyclase ribonucleic acid sequence or endogenous β-hydroxylase ribonucleic acid sequence (or a part thereof) in sense orientation can lead to cosuppression of the corresponding ε-cyclase gene or endogenous β-hydroxylase gene. The expression of sense RNA with homology to an endogenous gene can decrease or switch off the expression thereof, in a similar manner to that which has been described for antisense arrangements (Jorgensen et al. (1996) Plant Mol Biol 31(5):957-973; Goring et al. (1991) Proc Natl Acad Sci USA 88:1770-1774; Smith et al. (1990) Mol Gen Genet 224:447-481; Napoli et al. (1990) Plant Cell 2:279-289; Van der Krol et al. (1990) Plant Cell 2:291-99). Here, the construct introduced can completely or only partially represent the homologous gene to be decreased. The possibility of translation is not necessary. The application of this technology to plants has been described (e.g. Napoli et al. (1990) Plant Cell 2:279-289) in U.S. Pat. No. 5,034,323.

Preferably, the cosuppression for the particularly preferred plant Tagetes erecta is realized using a sequence which is essentially identical to at least one part of the nucleic acid sequence coding for an ε-cyclase or endogenous β-hydroxylase, for example the nucleic acid sequence as set forth in SEQ ID NO: 7 or SEQ. ID. NO. 16.

Preferably, the sense RNA is chosen such that a translation of the corresponding protein or a part thereof cannot occur. For this, it is possible, for example, to choose the 5′-untranslated or 3′-untranslated region or else to delete or mutate the ATG start codon.
e) introduction of DNA- or protein-binding factors against ε-cyclase genes, RNAs or proteins or against endogenous β-hydroxylase genes, RNAs or proteins

A decrease in an ε-cyclase or β-hydroxylase expression is also possible with specific DNA-binding factors, e.g. with factors of the type consisting of the zinc-finger transcription factors. These factors are added to the genomic sequence of the endogenous target gene, preferably in the regulatory regions, and bring about a decrease in the expression. Corresponding processes for the preparation of appropriate factors have been described (Dreier B et al. (2001) J Biol Chem 276(31):29466-78; Dreier B et al. (2000) J Mol Biol 303(4):489-502; Beerli R R et al. (2000) Proc Natl Acad Sci USA 97 (4):1495-1500; Beerli R R et al. (2000) J. Biol Chem 275(42):32617-32627; Segal D J and Barbas C F 3rd. (2000) Curr Opin Chem Biol 4(1):34-39; Kang J S and Kim J S (2000) J Biol Chem 275(12):8742-8748; Beerli R R et al. (1998) Proc Natl Acad Sci USA 95(25):14628-14633; Kim J S et al. (1997) Proc Natl Acad Sci USA 94(8):3616-3620; Klug A (1999) J. Mol. Biol. 293(2):215-218; Tsai S Y et al. (1998) Adv Drug Deliv Rev 30(1-3):23-31; Mapp A K et al. (2000) Proc Natl Acad Sci USA 97(8):3930-3935; Sharrocks A D et al. (1997) nt J Biochem Cell Biol 29(12):1371-1387; Zhang L et al. (2000) J Biol Chem 275(43):33850-33860).

The selection of these factors can be carried out using any desired piece of an ε-cyclase gene or endogenous β-hydroxylase gene. Preferably, this segment lies in the area of the promoter region. For gene suppression, it can, however, also lie in the area of the coding exons or introns.

Furthermore, factors can be introduced into a cell which inhibit the ε-cyclase or endogenous β-hydroxylase itself. These protein-binding factors can be, for example, aptamers (Famulok M and Mayer G (1999) Curr Top Microbiol Immunol 243:123-36) or antibodies or antibody fragments or single-chain antibodies. The obtainment of these factors has been described (Oweh M et al. (1992) Biotechnology (N Y) 10(7):790-794; Franken E et al. (1997) Curr Opin Biotechnol 8(4):411-416; Whitelam (1996) Trend Plant Sci 1:286-272).
f) introduction of the viral nucleic acid sequences and expression constructs bringing about ε-cyclase RNA degradation or endogenous β-hydroxylase RNA degradation

The ε-cyclase or endogenous β-hydroxylase expression can effectively also be realized by induction of specific RNA degradation by the plant with the aid of a viral expression system (Amplikon; Angell S M et al. (1999) Plant J 20(3):357-362). These systems—also described as “VIGS” (viral induced gene silencing)—introduce into the plant nucleic acid sequences having homology to the transcript of an enzyme activity to be reduced by means of viral vectors.

Transcription is then switched off—presumably mediated by plant defence mechanisms against viruses. Appropriate techniques and processes have been described (Ratcliff F et al. (2001) Plant J 25(2):237-45; Fagard M and Vaucheret H (2000) Plant Mol Biol 43(2-3):285-93; Anandalakshmi R et al. (1998) Proc Natl Acad Sci USA 95(22):13079-84; Ruiz M T (1998) Plant Cell 10(6):937-46).

Preferably, the VIGS-mediated decrease is realized using a sequence which is essentially identical to at least a part of the nucleic acid sequence coding for an ε-cyclase or an endogenous β-hydroxylase, for example the nucleic acid sequence as set forth in SEQ ID NO: 7 or 16.
g) introduction of constructs for the production of a functional loss or of a functional decrease of ε-cyclase genes or endogenous β-hydroxylase genes

Numerous processes are known to the person skilled in the art by which genomic sequences can be specifically modified. These include, in particular, processes such as the production of knockout mutants by means of specific homologous recombination, e.g. by generation of stop codons, shifts in the reading frame etc. (Hohn B and Puchta H (1999) Proc Natl Acad Sci USA 96:8321-8323) or the specific deletion or inversion of sequences by means of, for example, sequence-specific recombinases or nucleases (see below).

The decrease in the amount of enzyme, enzyme function and/or activity can also be realized by a specific insertion of nucleic acid sequences (e.g. of the nucleic acid sequence to be inserted in the context of the process according to the invention) into the sequence coding for an ε-cyclase or endogenous β-hydroxylase (e.g. by means of intermolecular homologous recombination). In the context of this embodiment, a DNA construct is preferably used which comprises at least a part of the sequence of an ε-cyclase gene or endogenous β-hydroxylase gene or adjacent sequences, and can specifically be recombined with this in the target cell, such that by a deletion, addition or substitution of at least one nucleotide the ε-cyclase gene or endogenous β-hydroxylase gene is modified such that the functionality of the gene is reduced or entirely abolished.

The modification can also relate to the regulative elements (e.g. the promoter) of the genes such that the coding sequence remains unchanged, but expression (transcription and/or translation) does not occur or is reduced. In the case of conventional homologous recombination, the sequence to be inserted is flanked on its 5′ and/or 3′ end by further nucleic acid sequences (A′ or B′), which have an adequate length and homology to corresponding sequences of the ε-cyclase gene or endogenous β-hydroxylase gene (A or B) for the making possible of homologous recombination. The length is, as a rule, in a range from several hundred bases to several kilobases (Thomas K R and Capecchi M R (1987) Cell 51:503; Strepp et al. (1998) Proc Natl Acad Sci USA 95(8):4368-4373). For homologous recombination, the plant cell is transformed with the recombination construct using the process described below and successfully recombined clones based on the ε-cyclase or endogenous β-hydroxylase which is inactivated as a result are selected.

In a further preferred embodiment, the efficiency of recombination is increased by combination with processes which promote homologous recombination. Such processes have been described and comprise, by way of example, the expression of proteins such as RecA or treatment with PARP inhibitors. It was possible to show that intrachromosomal homologous recombination in tobacco plants can be increased by the use of PARP inhibitors (Puchta H et al. (1995) Plant J 7:203-210). By the use of these inhibitors, the rate of homologous recombination in the recombination constructs after induction of the sequence-specific DNA double-strand breakage and thus the efficiency of the deletion of the transgene sequences can be further increased. Various PARP inhibitors can be employed here. Preferably, inhibitors such as 3-amino-benzamide, 8-hydroxy-2-methylquinazolin-4-one (NU1025), 1,11b-dihydro-[2H]benzopyrano[4,3,2-de]isoquinolin-3-one (GPI 6150), 5-aminoisoquinolinone, 3,4-dihydro-5-[4-(1-piperidinyl)butoxy]-1(2H)-isoquinolinone or the substances described in WO 00/26192, WO 00/29384, WO 00/32579, WO 00/64878, WO 00/68206, WO 00/67734, WO 01/23386 and WO 01/23390 are included.

Further suitable methods are the introduction of nonsense mutations into endogenous marker protein genes, for example by means of introduction of RNA/DNA oligo-nucleotides into the plants (Zhu et al. (2000) Nat Biotechnol 18(5):555-558) or the generation of knockout mutants with the aid of, for example, T-DNA mutagenesis (Koncz et al., Plant Mol. Biol. 1992, 20(5):963-976). Point mutations can also be produced by means of DNA-RNA hybrids, which are also known as “chimeraplasty” (Cole-Strauss et al. (1999) Nucl Acids Res 27(5):1323-1330; Kmiec (1999) Gene therapy American Scientist 87(3):240-247).

In a particularly preferred embodiment of the process according to the invention, the reduction of the ε-cyclase activity compared to the wild-type is carried out by:
a) introduction of at least one double-stranded ε-cyclase ribonucleic acid sequence or an expression cassette or expression cassettes guaranteeing its expression in plants and/or
b) introduction of at least one ε-cyclase antisense ribonucleic acid sequence or an expression cassette guaranteeing its expression in plants.

In a very particularly preferred embodiment, the reduction of the ε-cyclase activity compared to the wild-type takes place by introduction of at least one double-stranded ε-cyclase ribonucleic acid sequence or an expression cassette or expression cassettes guaranteeing its expression in plants.

Preferably, the transcription of the ε-cyclase dsRNA sequences in the process according to the invention takes place under the control of regulation signals, preferably a promoter and plastidic transit peptides, which guarantee the transcription of the ε-cyclase dsRNA sequences in the plant tissues containing photosynthetically inactive plastids.

In a particularly preferred embodiment, genetically modified plants are used which, in plant tissues comprising photosynthetically inactive plastids, have the highest transcription rate of the ε-cyclase dsRNA sequences.

Preferably, this is achieved by carrying out the transcription of the ε-cyclase dsRNA sequences under the control of a promoter specific for the plant tissue.

For the case described above where the expression is to take place in flowers, it is advantageous that the transcription of the ε-cyclase dsRNA sequences takes place under the control of a flower-specific or preferably petal-specific promoter.

For the case described above where the expression is to take place in fruits, it is advantageous that the transcription of the ε-cyclase dsRNA sequences takes place under the control of a fruit-specific promoter.

For the case described above where the expression is to take place in tubers, it is advantageous that the transcription of the ε-cyclase dsRNA sequences takes place under the control of a tuber-specific promoter.

In a particularly preferred embodiment of the process according to the invention, the reduction of the endogenous β-hydroxylase activity compared to the wild-type is carried out by:
a) introduction of at least one double-stranded endogenous β-hydroxylase ribonucleic acid sequence or an expression cassette or expression cassettes guaranteeing its expression in plants and/or
b) introduction of at least one endogenous β-hydroxylase antisense ribonucleic acid sequence or an expression cassette guaranteeing its expression in plants.

In a very particularly preferred embodiment, the reduction of the endogenous β-hydroxylase activity compared with the wild-type is carried out by introduction of at least one double-stranded endogenous β-hydroxylase ribonucleic acid sequence or an expression cassette or expression cassettes guaranteeing its expression in plants.

Preferably, the transcription of the endogenous β-hydroxylase dsRNA sequences in the process according to the invention is carried out under the control of regulation signals, preferably a promoter and plastidic transit peptides, which guarantee the transcription of the endogenous β-hydroxylase dsRNA sequences in the plant tissues comprising photosynthetically inactive plastids.

In a particularly preferred embodiment, genetically modified plants are used which, in plant tissues comprising photosynthetically inactive plastids, have the highest transcription rate of the endogenous β-hydroxylase dsRNA sequences.

Preferably, this is achieved by carrying out the transcription of the endogenous β-hydroxylase dsRNA sequences under the control of a promoter specific for the plant tissue.

For the case described above where the expression is to take place in flowers, it is advantageous that the transcription of the endogenous β-hydroxylase dsRNA sequences takes place under the control of a flower-specific or more preferably petal-specific promoter.

For the case described above where the expression is to take place in fruits, it is advantageous that the transcription of the endogenous β-hydroxylase dsRNA sequences takes place under the control of a fruit-specific promoter.

For the case described above where the expression is to take place in tubers, it is advantageous that the transcription of the endogenous β-hydroxylase dsRNA takes place under the control of a tuber-specific promoter.

Particularly preferably, genetically modified plants having the following combinations of genetic modifications are used in the process according to the invention:

Genetically modified plants which in comparison to the wild-type have an increased β-cyclase activity according to the invention and an increased hydroxylase activity, genetically modified plants which in comparison to the wild-type have an increased β-cyclase activity according to the invention and a reduced ε-cyclase activity, genetically modified plants which in comparison to the wild-type have an increased β-cyclase activity according to the invention and a reduced, endogenous β-hydroxylase activity,
genetically modified plants which in comparison to the wild-type have an increased β-cyclase activity according to the invention, an increased hydroxylase activity and a reduced ε-cyclase activity,
genetically modified plants which in comparison to the wild-type have an increased cyclase activity according to the invention, a reduced ε-cyclase activity and a reduced, endogenous β-hydroxylase activity,
genetically modified plants which in comparison to the wild-type have an increased β-cyclase activity according to the invention, an increased hydroxylase activity and a reduced, endogenous β-hydroxylase activity,
genetically modified plants which in comparison to the wild-type have an increased β-cyclase activity according to the invention, an increased hydroxylase activity and a reduced ε-cyclase activity and a reduced, endogenous β-hydroxylase activity.

The production of these genetically modified plants can, as described below, be carried out, for example, by introducing individual nucleic acid constructs (expression cassettes) or by introducing multiple constructs, which contain up to two, three or four of the activities described.

In the process according to the invention for the preparation of β-carotenoids, the step of culturing the genetically modified plants, also called transgenic plants below, is preferably followed by harvesting of the plants and the isolation of the β-carotenoids from the plants or the plant tissues comprising photosynthetically inactive plastids.

The transgenic plants are raised on nutrient media in a manner known per se and suitably harvested.

The isolation of β-carotenoids from the harvested plant tissues comprising photosynthetically inactive plastids, such as, for example, flowers, fruits or tubers, is carried out in a manner known per se, for example by drying and subsequent extraction and optionally further chemical or physical purification processes, such as, for example, precipitation methods, crystallography, thermal separation processes, such as rectification processes, or physical separation processes, such as, for example, chromatography. The isolation of β-carotenoids from the plant tissues comprising photosynthetically inactive plastids, such as, for example, flowers, fruits or tubers is carried out, for example, preferably by means of organic solvents such as acetone, hexane, ether or tert-methyl butyl ether.

Further isolation processes of β-carotenoids, in particular from petals, are described, for example, in Egger and Kleinig (Phytochemistry (1967) 6, 437-440) and Egger (Phytochemistry (1965) 4, 609-618).

Preferably, the β-carotenoids are selected from the group consisting of β-carotene, β-cryptoxanthin, zeaxanthin, antheraxanthin, violaxanthin and neoxanthin.

Preferred α-carotenoids are β-carotene and zeaxanthin, particularly preferably zeaxanthin.

Preferred plant tissues are those comprising photosynthetically inactive plastids, selected from the group consisting of flower, fruit and tuber.

In a preferred embodiment of the process according to the invention, the genetically modified plant used which in comparison to the wild-type has an increased β-cyclase activity in flowers is a plant selected from the families Ranunculaceae, Berberidaceae, Papaveraceae, Cannabaceae, Rosaceae, Fabaceae, Linaceae, Vitaceae, Brassiceae, Cucurbitaceae, Primulaceae, Caryophyllaceae, Amaranthaceae, Gentianaceae, Geraniaceae, Caprifoliaceae, Oleaceae, Tropaeolaceae, Solanaceae, Scrophulariaceae, Asteraceae, Liliaceae, Amaryllidaceae, Poaceae, Orchidaceae, Malvaceae, Illiaceae or Lamiaceae.

Particularly preferred plants are those selected from the plant genera Marigold, Tagetes erecta, Tagetes patula, Acacia, Aconitum, Adonis, Arnica, Aqulegia, Aster, Astragalus, Bignonia, Calendula, Calendula officinalis, Caltha, Campanula, Canna, Centaurea, Cheiranthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hisbiscus, Heliopsis, Hypericum, Hypochoeris, Impatiens, Iris, Jacaranda, Kerria, Laburnum, Lathyrus, Leontodon, Lilium, Linum, Lotus, Lycopersicon, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Solanum tuberosum, Sorbus, Spartium, Tecoma, Torenia, Tragopogon, Trollius, Tropaeolum, Tulipa, Tussilago, Ulex, Viola or Zinnia.

In a further preferred embodiment of the process according to the invention, the genetically modified plant used which, in comparison to the wild-type, has an increased β-cyclase activity in fruit, is a plant selected from the plant genera Actinophloeus, Aglaeonema, Ananas, Arbutus, Archontophoenix, Area, Aronia, Asparagus, Avocado, Attalea, Berberis, Bixia, Brachychilum, Bryonia, Caliptocalix, Capsicum, Carica, Celastrus, Citrullus, Citrus, Convallaria, Cotoneaster, Crataegus, Cucumis, Cucurbita, Cuscuta, Cycas, Cyphomandra, Dioscorea, Diospyrus, Dura, Elaeagnus, Elaeis, Erythroxylon, Euonymus, Erbse, Ficus, Fortunella, Fragaria, Gardinia, Gonocaryum, Gossypium, Guava, Guilielma, Hibiscus, Hippophaea, Iris, Kiwi, Lathyrus, Lonicera, Luffa, Lycium, Lycopersicum, Mais, Malpighia, Mangifera, Mormodica, Murraya, Musa, Nenga, Orange, Palisota, Pandanus, Passiflora, Persea, Physalis, Prunus, Ptychandra, Punica, Pyracantha, Pyrus, Ribes, Rosa, Rubus, Sabal, Sambucus, Seaforita, Shepherdia, Solanum, Sorbus, Synaspadix, Tabemae, Tamus, Taxus, Trichosanthes, Triphasia, Vaccinium, Viburnum, Vignia, Vitis or Zucchini.

In a further preferred embodiment of the process according to the invention, the genetically modified plant used which, in comparison to the wild-type, has an increased β-cyclase activity in tubers, is a plant selected from the plant genera red beet, radishes and Solanum tuberosum.

Particularly preferred plants have, as the wild-type, a higher proportion of α-carotenoids than β-carotenoids in the total carotenoid content in plant tissues comprising photosynthetically inactive plastids.

Particularly preferred plants are Marigold, Tagetes erecta, Tagetes patula where the preparation of the β-carotenoids, preferably zeaxanthin, takes place in flowers, particularly preferably in the petals.

Below, the production of genetically modified plants having increased β-cyclase activity in plant tissues comprising photosynthetically inactive plastids, such as, for example, flowers, fruits or tubers, is described as an example. The increase in further activities, such as, for example, the hydroxylase activity can be carried out analogously using nucleic acid sequences encoding a hydroxylase instead of nucleic acid sequences encoding a β-cyclase. The reduction of further activities, such as, for example, the reduction of the ε-cyclase activity and/or the endogenous β-hydroxylase activity can be carried out analogously using antisense nucleic acid sequences or inverted-repeat nucleic acid sequences instead of nucleic acid sequences encoding a β-cyclase.

The transformation can be carried out individually in the combinations of genetic modifications or by means of multiple constructs.

The transgenic plants are preferably produced by transformation of the starting plants, using a nucleic acid construct which comprises the nucleic acids described above encoding a β-cyclase, which are functionally linked with one or more regulation signals which guarantee transcription and translation in plants.

These nucleic acid constructs, in which the coding nucleic acid sequences are functionally linked with one or more regulation signals which guarantee transcription and translation in plants, are also called expression cassettes below.

The invention furthermore relates to nucleic acid constructs containing at least one nucleic acid encoding a β-cyclase and additionally at least one further nucleic acid, selected from the group consisting of
a) nucleic acids encoding a β-hydroxylase,
b) double-stranded endogenous β-hydroxylase ribonucleic acid sequence and/or endogenous β-hydroxylase antisense ribonucleic acid sequences and
c) double-stranded ε-cyclase ribonucleic acid sequence and/or ε-cyclase antisense ribonucleic acid sequence,
   the nucleic acids being functionally linked with one or more regulation signals which guarantee transcription and translation in plants.

It is, in particular in plants, technically only possible with difficulty to realize an increase in or a lowering of a number of activities using a nucleic acid construct. Therefore, combinations of nucleic acid constructs are preferably used in order to increase or to lower the activities, in particular by more than 3 activities, in plants.

It is, however, also possible to cross genetically modified plants which already comprise modified activities. For example, it is possible by crossing genetically modified plants which in each case comprise two modified activities to produce plants having four modified activities. Same can also be achieved by introducing a combination of two nucleic acid constructs which in each case modify 2 activities in the plants.

In a preferred embodiment, the preferred genetically modified plants are produced by introducing combinations of nucleic acid constructs.

Preferably, the regulation signals comprise one or more promoters, which guarantee transcription and translation in plant tissues comprising photosynthetically inactive plastids, such as, for example, flowers, fruits or tubers.

The expression cassettes contain regulation signals, that is regulative nucleic acid sequences, which control the expression of the coding sequence in the host cell. According to a preferred embodiment, an expression cassette comprises upstream, i.e. at the 5′ end of the coding sequence, a promoter and downstream, i.e. at the 3′ end, a polyadenylation signal and optionally further regulatory elements, which are operatively linked with the intermediate coding sequence for at least one of the genes described above. Operative linkage is understood as meaning the sequential arrangement of promoter, coding sequence, terminator and optionally further regulative elements in such a way that each of the regulative elements can fulfill its function in the expression of the coding sequence as intended.

Below, the preferred nucleic acid constructs, expression cassettes and vectors for plants, and processes for the production of transgenic plants, and the transgenic plants themselves are described by way of example.

The sequences preferred for the operative linkage but not restricted thereto are targeting sequences for guaranteeing the subcellular location in the apoplast, in the vacuole, in plastids, in the mitochondrium, in the endoplasmic reticulum (ER), in the cell nucleus, in elaioplasts or other compartments and translation amplifiers such as the 5′-guide sequence from the tobacco mosaic virus (Gallie et al., Nucl. Acids Res. 15 (1987), 8693-8711).

A suitable promoter according to the invention of the expression cassette is fundamentally any promoter which can control the expression of foreign genes in plant tissues comprising photosynthetically inactive plastids, such as, for example, flower, fruit or tuber.

“Constitutive” promoter means those promoters which guarantee expression in numerous, preferably all, tissues over a relatively large period of development of the plant, preferably at all times in the development of the plant.

Preferably, a plant promoter or a promoter is used in particular which originates from a plant virus. In particular, the promoter of the 35S transcript of the CaMV cauliflower mosaic virus (Franck et al. (1980) Cell 21:285-294; Odell et al. (1985) Nature 313:810-812; Shewmaker et al. (1985) Virology 140:281-288; Gardner et al. (1986) Plant Mol Biol 6:221-228) or der 19S CaMV promoter (U.S. Pat. No. 5,352,605; WO 84/02913; Benfey et al. (1989) EMBO J 8:2195-2202) is preferred.

A further suitable constitutive promoter is the pds promoter (Pecker et al. (1992) Proc. Natl. Acad. Sci USA 89: 4962-4966) or the “Rubisco small subunit (SSU)” promoter (U.S. Pat. No. 4,962,028), the Legumin B promoter (GenBank Acc. No. X03677), the promoter of nopaline synthase from Agrobacterium, the TR double promoter, der OCS (octopine synthase) promoter from Agrobacterium, the ubiquitin promoter (Holtorf S et al. (1995) Plant Mol Biol 29:637-649), the ubiquitin 1 promoter (Christensen et al. (1992) Plant Mol Biol 18:675-689; Bruce et al. (1989) Proc Natl Acad Sci USA 86:9692-9696), the Smas promoter, the cinnamyl alcohol dehydrogenase promoter (U.S. Pat. No. 5,683,439), the promoters of vacuolar ATPase subunits or the promoter of a proline-rich protein from wheat (WO 91/13991), the Pnit promoter (Y07648.L, Hillebrand et al. (1998), Plant. Mol. Biol. 36, 89-99, Hillebrand et al. (1996), Gene, 170, 197-200, the ferredoxin NADPH oxidoreductase promoter (database entry AB011474, position 70127 to 69493), the TPT promoter (WO 03006660), the “Superpromotor” (U.S. Pat. No. 5,955,646), the 34S promoter (U.S. Pat. No. 6,051,753), and further promoters of genes whose constitutive expression in plants is known to the person skilled in the art.

The expression cassettes can also comprise a chemically inducible promoter (review article: Gatz et al. (1997) Annu Rev Plant Physiol Plant Mol Biol 48:89-108), by means of which the expression of the β-cyclase gene in the plant can be controlled at a certain time. Promoters of this type, such as, for example, the PRP1 promoter (Ward et al. (1993) Plant Mol Biol 22:361-366), a promoter inducible by salicylic acid (WO 95/19443), a promoter inducible by benzenesulfonamide (EP 0 388 186), a promoter inducible by tetracycline (Gatz et al. (1992) Plant J 2:397-404), a promoter inducible by abscisic acid (EP 0 335 528) or a promoter inducible by ethanol or cyclohexanone (WO 93/21334) can likewise be used.

Furthermore, promoters are preferred which are induced by biotic or abiotic stress such as, for example, the pathogen-inducible promoter of the PRP1 gene (Ward et al. (1993) Plant Mol Biol 22:361-366), the heat-inducible hsp70 or hsp80 promoter from tomato (U.S. Pat. No. 5,187,267), the cold-inducible alpha-amylase promoter from the potato (WO 96/12814), the light-inducible PPDK promoter or the wounding-induced pinII promoter (EP375091).

Pathogen-inducible promoters include those from genes which are induced as a result of a pathogen attack such as, for example, genes of PR proteins, SAR proteins, β-1,3-glucanase, chitinase etc. (for example Redolfi et al. (1983) Neth J Plant Pathol 89:245-254; Uknes, et al. (1992) The Plant Cell 4:645-656; Van Loon (1985) Plant Mol Viral 4:111-116; Marineau et al. (1987) Plant Mol Biol 9:335-342; Matton et al. (1987) Molecular Plant-Microbe Interactions 2:325-342; Somssich et al. (1986) Proc Natl Acad Sci USA 83:2427-2430; Somssich et al. (1988) Mol Gen Genetics 2:93-98; Chen et al. (1996) Plant J 10:955-966; Zhang and Sing (1994) Proc Natl Acad Sci USA 91:2507-2511; Warner, et al. (1993) Plant J 3:191-201; Siebertz et al. (1989) Plant Cell 1:961-968(1989).

Also included are wounding-inducible promoters such as that of the pinII gene (Ryan (1990) Ann Rev Phytopath 28:425-449; Duan et al. (1996) Nat Biotech. 14:494-498), of the wun1 and wun2 gene (U.S. Pat. No. 5,428,148), of the win1 and win2 gene (Stanford et al. (1989) Mol Gen Genet 215:200-208), of systemin (McGurl et al. (1992) Science 225:1570-1573), of the WIP1 gene (Rohmeier et al. (1993) Plant Mol Biol 22:783-792; Ekelkamp et al. (1993) FEBS Letters 323:73-76), of the MPI gene (Corderok et al. (1994) The Plant J 6(2):141-150) and the like.

Further suitable promoters are, for example, fruit ripening-specific promoters, such as, for example the fruit ripening-specific promoter from tomato (WO 94/21794, EP 409 625). Development-dependent promoters in some cases include the tissue-specific promoters, since the formation of individual tissue naturally takes place in a development-dependent manner.

Furthermore, those promoters are in particular preferred which ensure expression in tissues or plant tissues, in which, for example, the biosynthesis of β-carotenoids or their precursors takes place. Preferred promoters are, for example, those with specificities for the anthers, ovaries, petals, sepals, flowers, leaves, stalks and roots and combinations hereof.

Tuber-, storage root- or root-specific promoters are, for example, the class I patatin promoter (B33) or the promoter of cathepsin D inhibitor from potato.

Flower-specific promoters are, for example, the phytoene synthase promoter (WO 92/16635), the promoter of the P-rr gene (WO 98/22593), the EPSPS promoter (M37029), the DFR-A promoter (X79723), the B-gene promoter (WO 0008920) and the CHRC promoter (WO 98/24300; Vishnevetsky et al. (1996) Plant J. 10, 1111-1118), the promoter P76 and P84 (DE patent application 10247599.7), and the promoters of the Arabidopsis gene loci At5g33370 (as a result of M1 promoter), At5g22430 (as a result of M2 promoter) and At1g26630 (as a result of M3 promoter).

Further promoters suitable for expression in plants are described in Rogers et al. (1987) Methods in Enzymol 153:253-277; Schardl et al. (1987) Gene 61:1-11 and Berger et al. (1989) Proc Natl Acad Sci USA 86:8402-8406).

All promoters described in the present application make possible the expression of the β-cyclase in plant tissues comprising photosynthetically inactive plastids, such as, for example, flower, fruit or tuber.

Preferred promoters are promoters which are specific for plant tissues comprising photosynthetically inactive plastids.

In the process according to the invention, as mentioned above, depending on the plant used, constitutive, flower-specific and in particular floral leaf-specific, fruit specific and tuber-specific promoters are particularly preferred.

The present invention therefore in particular relates to a nucleic acid construct, comprising, functionally linked, a flower-specific or in particular a floral leaf-specific promoter and a nucleic acid encoding a β-cyclase, comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2.

The present invention therefore in particular relates to a nucleic acid construct, comprising, functionally linked, a fruit-specific promoter and a nucleic acid encoding a β-cyclase, comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, with the proviso that the natural promoter of the β-cyclase is excluded.

The present invention therefore in particular relates to a nucleic acid construct, comprising, functionally linked, a tuber-specific promoter and a nucleic acid encoding a β-cyclase, comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, with the proviso that the natural promoter of the β-cyclase is excluded.

The present invention therefore in particular relates to a nucleic acid construct, comprising, functionally linked, a constitutive promoter and a nucleic acid encoding a β-cyclase, comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, with the proviso that the natural promoter of the β-cyclase is excluded.

The preparation of an expression cassette is preferably carried out by fusion of a suitable promoter with a nucleic acid described above encoding a β-cyclase and preferably a nucleic acid inserted between promoter and nucleic acid sequence, which nucleic acid codes for a plastid-specific transit peptide, and a polyadenylation signal according to customary recombination and cloning techniques, such as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1987).

The preferably inserted nucleic acids encoding a plastid transit peptide guarantee the location in plastids and in particular in chromoplasts.

It is also possible to use expression cassettes whose nucleic acid sequence codes for a β-cyclase fusion protein, a part of the fusion protein being a transit peptide which controls the translocation of the polypeptide. For the chromoplasts, specific transit peptides are preferred which, after translocation of the β-cyclase in the chromoplasts of the β-cyclase part, are enzymatically cleaved.

In particular, the transit peptide is preferred which is derived from the plastidic Nicotiana tabacum transketolase or another transit peptide (e.g. the transit peptide of the small subunit of the Rubisco (rbcS) or ferredoxin NADP oxidoreductase and isopentenyl pyrophosphate isomerase-2) or its functional equivalent.

Particularly preferred nucleic acid sequences are those from three cassettes of the plastid transit peptide of the plastidic transketolase from tobacco in three reading frames as KpnI/BamHI fragments having an ATG codon in the NcoI cleavage site:

pTP09
KpnI_GGTACCATGGCGTCTTCTTCTTCTCTCACTCTCTCTCAAGCTATC
CTCTCTCGTTCTGTCCCTCGCCATGGCTCTGCCTCTTCTTCTCAACTTTC
CCCTTCTTCTCTCACTTTTTCCGGCCTTAAATCCAATCCCAATATCACCA
CCTCCCGCCGCCGTACTCCTTCCTCCGCCGCCGCCGCCGCCGTCGTAAGG
TCACCGGCGATTCGTGCCTCAGCTGCAACCGAAACCATAGAGAAAACTGA
GACTGCGGGATCC_BamHI
pTP10
KpnI_GGTACCATGGCGTCTTCTTCTTCTCTCACTCTCTCTCAAGCTATC
CTCTCTCGTTCTGTCCCTCGCCATGGCTCTGCCTCTTCTTCTCAACTTTC
CCCTTCTTCTCTCACTTTTTCCGGCCTTAAATCCAATCCCAATATCACCA
CCTCCCGCCGCCGTACTCCTTCCTCCGCCGCCGCCGCCGCCGTCGTAAGG
TCACCGGCGATTCGTGCCTCAGCTGCAACCGAAACCATAGAGAAAACTGA
GACTGCGCTGGATCC_BamH I
pTP11
KpnI_GGTACCATGGCGTCTTCTTCTTCTCTCACTCTCTCTCAAGCTATC
CTCTCTCGTTCTGTCCCTCGCCATGGCTCTGCCTCTTCTTCTCAACTTTC
CCCTTCTTCTCTCACTTTTTCCGGCCTTAAATCCAATCCCAATATCACCA
CCTCCCGCCGCCGTACTCCTTCCTCCGCCGCCGCCGCCGCCGTCGTAAGG
TCACCGGCGATTCGTGCCTCAGCTGCAACCGAAACCATAGAGAAAACTGA
GACTGCGGGGATCC_BamHI.

Further examples of a plastid transit peptide are the transit peptide of the plastidic isopentenyl pyrophosphate isomerase-2 (IPP-2) from Arabisopsis thaliana and the transit peptide of the small subunit of ribulose bisphosphate carboxylase (rbcS) from pea (Guerineau, F, Woolston, S, Brooks, L, Mullineaux, P (1988) An expression cassette for targeting foreign proteins into the chloroplasts. Nucl. Acids Res. 16: 11380).

The nucleic acids according to the invention can be prepared synthetically or obtained naturally or comprise a mixture of synthetic and natural nucleic acid constituents, and consist of various heterologous gene segments of various organisms.

As described above, synthetic nucleotide sequences with codons which are preferably from plants are preferred. These preferred codons from plants can be determined from codons having the highest protein frequency, which are expressed in the most interesting plant species.

In the preparation of an expression cassette, various DNA fragments can be manipulated in order to obtain a nucleotide sequence which expediently reads in the correct direction and which is equipped with a correct reading frame. For the connection of the DNA fragments to one another, adapters or linkers can be attached to the fragments.

Expediently, the promoter and the terminator regions can be provided in the transcription direction with a linker or polylinker which comprises one or more restriction sites for the insertion of this sequence. As a rule, the linker has 1 to 10, usually 1 to 8, preferably 2 to 6 restriction sites. In general, the linker within the regulatory regions has a size of less than 100 bp, often less than 60 bp, but at least 5 bp. The promoter can be either native or homologous and alien or homologous for the host plant. The expression cassette preferably contains in the 5′-3′ transcription direction the promoter, a coding nucleic acid sequence or a nucleic acid construct and a region for transcriptional termination. Various termination regions are arbitrarily mutually exchangeable.

Examples of a terminator are the 35S terminator (Guerineau et al. (1988) Nucl Acids Res. 16: 11380), the nos terminator (Depicker A, Stachel S, Dhaese P, Zambryski P, Goodman H M. Nopaline synthase: transcript mapping and DNA sequence. J Mol Appl Genet. 1982; 1 (6):561-73) or the ocs terminator (Gielen, J, de Beuckeleer, M, Seurinck, J, Debroek, H, de Greve, H, Lemmers, M, van Montagu, M, Schell, J (1984) The complete sequence of the TL-DNA of the Agrobacterium tumefaciens plasmid pTiAch5. EMBO J. 3: 835-846).

Furthermore, manipulations which produce suitable restriction cleavage sites or remove the superfluous DNA or restriction cleavage sites can be employed. Where insertions, deletions or substitutions such as, for example, transitions and transversions are possible, in vitro mutagenesis, “primer repair”, restriction or ligation can be used.

In the case of suitable manipulations, such as, for example, restriction, “chewing-back” or filling of overhangs for “blunt ends”, complementary ends of the fragments for ligation can be made available.

Preferred polyadenylation signals are plant polyadenylation signals, preferably those which correspond essentially to T-DNA polyadenylation signals from Agrobacterium tumefaciens, in particular of gene 3 of the T-DNA (octopin synthase) of the Ti plasmid pTiACH5 (Gielen et al., EMBO J. 3 (1984), 835 ff) or functional equivalents.

The transfer of foreign genes to the genome of a plant is designated as transformation.

To this end, methods known per se for the transformation and regeneration of plants from plant tissues or plant cells can be used for transient or stable transformation.

Suitable methods for the transformation of plants are protoplast transformation by polyethylene glycol-induced DNA uptake, the biolistic process using the gene gun—the “particle bombardment method”, electroporation, the incubation of dry embryos in DNA-containing solution, microinjection and the gene transfer described above, mediated by Agrobacterium. The processes mentioned are described, for example, in B. Jenes et al., Techniques for Gene Transfer, in: Transgenic Plants, Vol. 1, Engineering and Utilization, edited by S. D. Kung and R. Wu, Academic Press (1993), 128-143, and in Potrykus, Annu. Rev. Plant Physiol. Plant Molec. Biol. 42 (1991), 205-225).

Preferably, the construct to be expressed is cloned into a vector which is suitable for transforming Agrobacterium tumefaciens, for example pBin19 (Bevan et al., Nucl. Acids Res. 12 (1984), 8711) or particularly preferably pSUN2, pSUN3, pSUN4 or pSUN5 (WO 02/00900).

Agrobacteria transformed with an expression plasmid can be used in a known manner for the transformation of plants, e.g. by bathing wounded leaves or pieces of leaf in an Agrobacteria solution and subsequently culturing in suitable media.

For the preferred production of genetically modified plants, also called transgenic plants below, the fused expression cassette, which expresses a β-cyclase, is cloned into a vector, for example pBin19 or in particular pSUN5, which is suitable to be transformed to Agrobacterium tumefaciens. Agrobacteria transformed using such a vector can then be used in a known manner for the transformation of plants, in particular of cultured plants, by, for example, bathing wounded leaves or pieces of leaf in an Agrobacteria solution and subsequently culturing in suitable media.

The transformation of plants by Agrobacteria is known, inter alia, from F. F. White, Vectors for Gene Transfer in Higher Plants; in Transgenic Plants, Vol. 1, Engineering and Utilization, edited by S. D. Kung and R. Wu, Academic Press, 1993, pp. 15-38. From the transformed cells of the wounded leaves or pieces of leaf, it is possible in a known manner to regenerate transgenic plants which comprise a gene integrated into the expression cassette for the expression of a nucleic acid encoding a β-cyclase.

For the transformation of a host plant using a nucleic acid coding for a β-cyclase, an expression cassette is incorporated into a recombinant vector as an insertion whose vector DNA comprises additional functional regulation signals, for example sequences for replication or integration. Suitable vectors are described, inter alia, in “Methods in Plant Molecular Biology and Biotechnology” (CRC Press), Kap. 6/7, pp. 71-119 (1993).

Using the recombination and cloning techniques cited above, the expression cassettes can be cloned into suitable vectors which make possible their replication, for example in E. coli. Suitable cloning vectors are, inter alia, pJIT117 (Guerineau et al. (1988) Nucl. Acids Res. 16 :11380), pBR332, pUC series, M13 mp series and pACYC184. Binary vectors, which can replicate both in E. coli and in Agrobacteria, are particularly suitable.

The invention further relates to the genetically modified plants, where the genetic modification increases the activity of a β-cyclase in plant tissues comprising photosynthetically inactive plastids, compared to the wild-type, and the increased β-cyclase activity is caused by a β-cyclase comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2.

Preferably, the increase in the β-cyclase activity is carried out by an increase in the gene expression of a nucleic acid, encoding a β-cyclase, comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, compared to the wild-type.

Preferably, the increase in the gene expression is carried out by incorporating nucleic acids into the plant, encoding the β-cyclases comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2.

Preferred genetically modified plants are those which comprise at least one nucleic acid, encoding a β-cyclase, comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, with the proviso that tomato is excluded.

Further particularly preferred, genetically modified plants additionally have, as mentioned above, an increased hydroxylase activity compared to the wild-type. Further preferred embodiments are described above in the process according to the invention.

Further particularly preferred, genetically modified plants additionally have, as mentioned above, a reduced activity compared to the wild-type, at least one of the activities selected from the group consisting of ε-cyclase activity and endogenous β-hydroxylase activity. Further preferred embodiments are described above in the process according to the invention.

Preferably, the plant tissues comprising photosynthetically inactive plastids are selected from the group consisting of flower, fruit and tuber.

In a preferred embodiment, the genetically modified plants which, in comparison to the wild-type, have an increased β-cyclase activity in flowers, are selected from the families Ranunculaceae, Berberidaceae, Papaveraceae, Cannabaceae, Rosaceae, Fabaceae, Linaceae, Vitaceae, Brassiceae, Cucurbitaceae, Primulaceae, Caryophyllaceae, Amaranthaceae, Gentianaceae, Geraniaceae, Caprifoliaceae, Oleaceae, Tropaeolaceae, Solanaceae, Scrophulariaceae, Asteraceae, Liliaceae, Amaryllidaceae, Poaceae, Orchidaceae, Malvaceae, Illiaceae or Lamiaceae.

Particularly preferred plants are those selected from the plant genera Marigold, Tagetes erecta, Tagetes patula, Acacia, Aconitum, Adonis, Arnica, Aqulegia, Aster, Astragalus, Bignonia, Calendula, Calendula officinalis, Caltha, Campanula, Canna, Centaurea, Cheiranthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hisbiscus, Heliopsis, Hypericum, Hypochoeris, Impatiens, Iris, Jacaranda, Kerria; Laburnum, Lathyrus, Leontodon, Lilium, Linum, Lotus, Lycopersicon, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Solanum tuberosum, Sorbus, Spartium, Tecoma, Torenia, Tragopogon, Trollius, Tropaeolum, Tulipa, Tussilago, Ulex, Viola or Zinnia.

In a further preferred embodiment; the genetically modified plants which, in comparison to the wild-type, have an increased β-cyclase activity in fruits, are selected from the plant genera Actinophloeus, Aglaeonema, Ananas, Arbutus, Archontophoenix, Area, Aronia, Asparagus, Avocado, Attalea, Berberis, Bixia, Brachychilum, Bryonia, Caliptocalix, Capsicum, Carica, Celastrus, Citrullus, Citrus, Convallaria, Cotoneaster, Crataegus, Cucumis, Cucurbita, Cuscuta, Cycas, Cyphomandra, Dioscorea, Diospyrus, Dura, Elaeagnus, Elaeis, Erythroxylon, Euonymus, Erbse, Ficus, Fortunella, Fragaria, Gardinia, Gonocaryum, Gossypium, Guava, Guilielma, Hibiscus, Hippophaea, Iris, Kiwi, Lathyrus, Lonicera, Luffa, Lycium, Lycopersicum, Mais, Malpighia, Mangifera, Mormodica, Murraya, Musa, Nenga, Orange, Palisota, Pandanus, Passiflora, Persea, Physalis, Prunus, Ptychandra, Punica, Pyracantha, Pyrus, Ribes, Rosa, Rubus, Sabal, Sambucus, Seaforita, Shepherdia, Solanum, Sorbus, Synaspadix, Tabernae, Tamus, Taxus, Trichosanthes, Triphasia, Vaccinium, Viburnum, Vignia, Vitis or Zucchini.

In a further preferred embodiment, the genetically modified plants which, in comparison to the wild-type, have an increased β-cyclase activity in tubers, are Solanum tuberosum.

Particularly preferred plants have, as the wild-type, a higher proportion of α-carotenoids than α-carotenoids in the total carotenoid content in plant tissues comprising photosynthetically inactive plastids.

Particularly preferred genetically modified plants are those of the genus Tagetes, comprising at least one nucleic acid encoding a β-cyclase comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2.

Particularly preferred plants are Marigold, Tagetes erecta, Tagetes patula where the production of the β-carotenoids, preferably zeaxanthin, takes place in flowers, particularly preferably in the petals.

Particularly preferred plant tissues comprising photosynthetically inactive plastids are the root tuber of Solanum tuberosum, the seed fruits of Zea Mais, the flower of Tagetes erecta and the flower of Calendula officinalis.

The genetically modified plants, their propagation material, and their plant cells, tissue or parts, in particular their floral leaves, tubers or fruits, are a further subject of the present invention.

The genetically modified plants can, as described above, be used for the production of β-carotenoids, in particular β-carotene and zeaxanthin.

Genetically modified plants according to the invention consumable by humans and animals, having an increased content of β-carotenoids can also be used, for example, directly or after processing known per se as foodstuffs or feedstuffs or feed and food supplements. Furthermore, the genetically modified plants can be used for the production of β-carotenoid-containing extracts of the plants and/or for the production of feed supplements and food supplements.

Zeaxanthin-containing extracts can be used for the pigmentation of animal products, in particular of the family Galiformes. The pigmentation is carried out by oral administration of the zeaxanthin-containing extracts which the respective animal correspondingly processes and were prepared for oral administration. Animal products are understood in particular as meaning skin, meat, feathers and egg yolks.

The genetically modified plants can also be used as decorative plants in the horticulture field.

The genetically modified plants have, in comparison to the wild-type, an increased content of β-carotenoids in plant tissues comprising photosynthetically inactive plastids.

An increased content of β-carotenoids is as a rule understood as meaning an increased content of total β-carotenoid.

An increased content of β-carotenoids is, however, also in particular understood as meaning a modified content of the preferred β-carotenoids, without the total carotenoid content necessarily having to be increased.

In a particularly preferred embodiment, the genetically modified plants according to the invention have, in comparison to the wild-type, an increased content of β-carotene or zeaxanthin, in particular zeaxanthin in plant tissues comprising photosynthetically inactive plastids.

An increased content is in this case also understood as meaning a created content of β-carotenoids, or β-carotene or zeaxanthin.

The invention is illustrated by the examples which now follow, but is not restricted to these:

General Experimental Conditions:

Sequence Analysis of Recombinant DNA

The sequencing of recombinant DNA molecules was carried out using a laser fluorescence DNA sequencer from Licor (marketing by MWG Biotech, Ebersbach) according to the method of Sanger (Sanger et al., Proc. Natl. Acad. Sci. USA 74 (1977), 5463-5467).

### EXAMPLE 1

### Preparation of Expression Vectors for the Flower-Specific Expression of the Chromoplast-Specific Lycopene β-Cyclase from Lycopersicon esculentum Under the Control of the Promoter P76

a) Isolation of Promoter P76 by Means of PCR Using Genomic DNA from Arabidopsis thaliana as a Matrix.

The oligonucleotide primers SEQ. ID. NO. 20 (P76for) and SEQ. ID. NO. 21 (P76rev) were used for this. The oligonucleotides were provided in the synthesis with a 5′ phosphate residue. The genomic DNA was isolated from Arabidopsis thaliana as described (Galbiati M et al. Funct. Integr. Genomics 2000, 20 1:25-34).

The PCR amplification was carried out as follows:
80 ng of genomic DNA
1× Expand Long Template PCR buffer
2.5 mM MgCl₂
350 μM each of dATP, dCTP, dGTP, dTTp
300 nM each of each primer
2.5 units of Expand Long Template polymerase
in a final volume of 25 μl

The following temperature program is used:
1 cycle of 120 sec at 94° C.
35 cycles at 94° C. for 10 sec, 48° C. for 30 sec and 68° C. for 3 min
1 cycle at 68° C. for 10 min

The PCR product (SEQ. ID. NO. 22) is purified using agarose gel electrophoresis and the 1032 bp fragment is isolated by gel elution.

The vector pSun5 is digested using the restriction endonuclease EcoRV and likewise purified by means of agarose gel electrophoresis and recovered by gel elution.

The purified PCR product is cloned into the vector treated in this way.

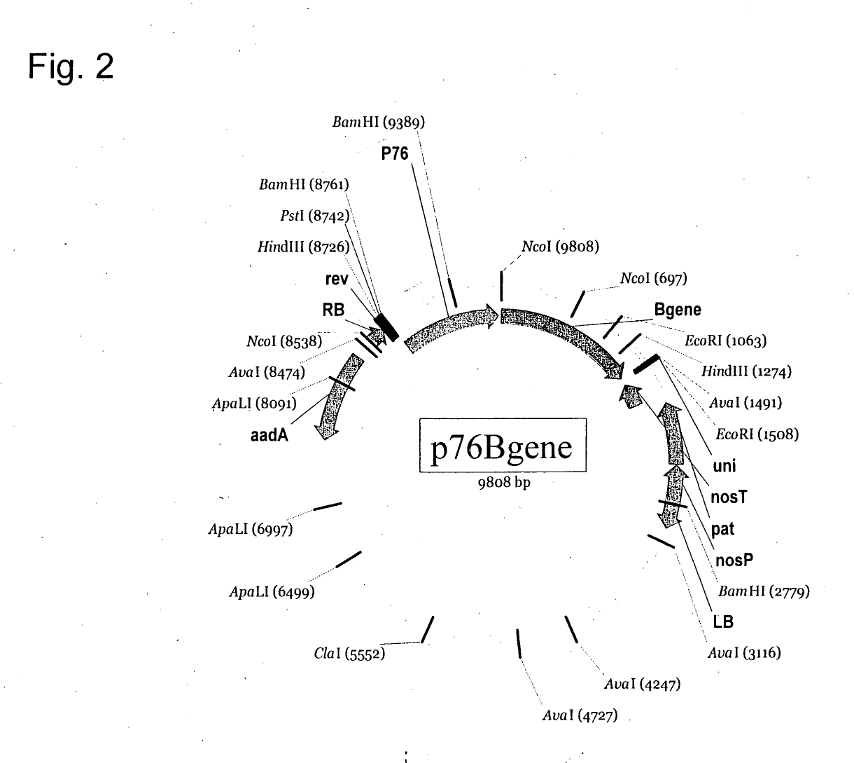

In order to check the orientation of the promoter in the vector it is digested using the restriction endonuclease BamHI. If a 628 bp fragment results here the orientation is according to FIG. 2.

This construct is indicated by p76.

b) Isolation of the Nucleic Acid Encoding a β-Cyclase (Bgene) by Means of PCR Using Genomic DNA from Lycopersicon esculentum as a Matrix.

The oligonucleotide primers SEQ. ID. NO. 23 (BgeneFor) and SEQ. ID. NO. 24 (BgeneRev) were used for this. The oligonucleotides were provided in the synthesis with a 5′ phosphate residue. The genomic DNA was isolated from Lycopersicon esculentum as described (Galbiati M et al. Funct. Integr. Genomics 2000, 20 1:25-34).

The PCR amplification was carried out as follows:
80 ng of genomic DNA
1× Expand Long Template PCR buffer
2.5 mM MgCl₂
350 μM each of dATP, dCTP, dGTP, dTTp
300 nM each of each primer
2.5 units of Expand Long Template polymerase
in a final volume of 25 μl

The following temperature programme was used:
1 cycle of 120 sec at 94° C.
35 cycles at 94° C. for 10 sec, 48° C. for 30 sec and 68° C. for 3 min
1 cycle at 68° C. for 10 min

The PCR product was purified using agarose gel electrophoresis and the 1486 bp fragment was isolated by gel elution.

The vector p76 is digested using the restriction endonuclease SmaI and likewise purified by means of agarose gel electrophoresis and recovered by gel elution.

The purified PCR product is cloned into the vector treated in this way.

In order to check the orientation of Bgene in the vector it is digested using the restriction endonuclease EcoRI. If a 445 bp fragment results here the orientation is according to FIG. 2.

This construct is indicated by p76Bgene.

### EXAMPLE 2

### Preparation of a Cloning Vector for the Preparation of Double-Stranded ε-Cyclase Ribonucleic Acid Sequence Expression Cassettes for the Flower-Specific Expression of Epsilon-Cyclase dsRNAs in Tagetes erecta

The expression of inverted-repeat transcripts consisting of fragments of the epsilon-cyclase in Tagetes erecta was carried out under the control of a modified version AP3P of the flower-specific promoter AP3 from Arabidopsis thaliana (AL132971: nucleotide region 9298-10200; Hill et al. (1998) Development 125: 1711-1721) The inverted-repeat transcript in each case comprises a fragment in correct orientation (sense fragment) and an identical sequence fragment in opposite orientation (antisense fragment), which are connected to one another by a functional intron, the PIV2 intron of the ST-LH1 gene from potato (Vancanneyt G. et al. (1990) Mol Gen Genet 220: 245-50).

The cDNA which codes for the AP3 promoter (−902 to +15) from Arabidopsis thaliana was prepared by means of PCR using genomic DNA (isolated from Arabidopsis thaliana according to a standard method) and the primers PR7 (SEQ ID No. 25) and PR10 (SEQ ID No. 28).

The PCR conditions were as follows:

The PCR for the amplification of the DNA which encodes the AP3 promoter fragment (−902 to +15) was carried out in a 50 μl reaction batch, which comprised:
1 μl of genomic DNA from A. thaliana (1:100 dil, prepared as described above)
0.25 mM dNTPs
0.2 mM PR7 (SEQ ID No. 25)
0.2 mM PR10 (SEQ ID No. 28)
5 μl of 10×PCR buffer (Stratagene)
0.25 μl of Pfu polymerase (Stratagene)
28.8 μl of dist. water

The PCR was carried out under the following cycle conditions:
1×94° C. 2 minutes
35×94° C. 1 minute
50° C. 1 minute
72° C. 1 minute
1×72° C. 10 minutes

The 922 bp amplificate was cloned into the PCR cloning vector pCR 2.1 (Invitrogen) using standard methods and the plasmid pTAP3 was obtained. Sequencing of the clone pTAP3 confirmed a sequence which only differed by an insertion (a G in position 9765 of the sequence AL132971) and a base exchange (a G instead of an A in position 9726 of the sequence AL132971) from the published AP3 sequence (AL132971, nucleotide region 9298-10200) (position 33: T instead of G, position 55: T instead of G). These nucleotide differences were reproduced in an independent amplification experiment and thus represent the nucleotide sequence in the Arabidopsis thaliana plant used.

The modified version AP3P was prepared by means of recombinant PCR using the plasmid pTAP3. The region 10200-9771 was amplified using the primers PR7 (SEQ ID No. 25) and PR9 (SEQ ID No. 27) (amplificate A7/9); the region 9526-9285 was amplified using the primers PR8 (SEQ ID No. 26) and PR10 (SEQ ID No. 28) (amplificate A8/10).

The PCR conditions were as follows:

The PCR reactions for the amplification of the DNA fragments which code for the regions 10200-9771 and 9526-9285 of the AP3 promoter were carried out in 50 μl reaction batches, which comprised:
100 ng of AP3 amplificate (described above)
0.25 mM dNTPs
0.2 mM PR7 (SEQ ID No. 15) or PR8 (SEQ ID No. 26)
0.2 mM PR9 (SEQ ID No. 17) or PR10 (SEQ ID No. 28)
5 ml 10×PCR buffer (Stratagene)
0.25 ml Pfu Taq polymerase (Stratagene)
28.8 ml of dist. water

The PCR was carried out under the following cycle conditions:
1×94° C. 2 minutes
35×94° C. 1 minute
50° C. 2 minutes
72° C. 3 minutes
1×72° C. 10 minutes.

The recombinant PCR comprises annealing of the amplificates A7/9 and A8/10 overlapping over a sequence of 25 nucleotides, completion to give a double strand and subsequent amplification. A modified version of the AP3 promoter, AP3P, thereby results in which the positions 9670-9526 are deleted. The denaturation (5 min at 95° C.) and annealing (slow cooling at room temperature to 40° C.) of both amplificates A7/9 and A8/10 resulted in a 17.6 ml reaction batch, which comprised:
0.5 mg of A7/9-
0.25 mg of A8/10

The filling of the 3′ ends (30 min at 30° C.) was carried out in a 20 μl reaction batch, which comprised:
17.6 ml of A7/9 and A8/10 annealing reaction (prepared as described above)
50 mM dNTPs
2 ml of 1× Klenow buffer
2 U of Klenow enzyme

The nucleic acid coding for the modified promoter version AP3P was amplified by means of PCR using a sense-specific primer (PR7 SEQ ID No. 25) and an antisense-specific primer (PR10 SEQ ID No. 28).

The PCR conditions were as follows:

The PCR for the amplification of the AP3P fragment was carried out in a 50 ml reaction batch, which comprised:
1 ml of annealing reaction (prepared as described above)
0.25 mM dNTPs
0.2 mM PR7 (SEQ ID No. 25)
0.2 mM PR10 (SEQ ID No. 28)
5 ml of 10×PCR buffer (Stratagene)
0.25 ml of Pfu Taq polymerase (Stratagene)
28.8 ml of dist. water

The PCR was carried out under the following cycle conditions:
1×94° C. 2 minutes
35×94° C. 1 minute
50° C. 1 minute
72° C. 1 minute
1×72° C. 10 minutes

The PCR amplification using PR7, SEQ ID No. 25 and PR10 SEQ ID No. 28 resulted in a 778 bp fragment which codes for the modified promoter version AP3P. The amplificate was cloned into the cloning vector pCR2.1 (Invitrogen). Sequences containing the primers T7 and M13 confirmed a sequence identical to the sequence AL132971, region 10200-9298, the internal region 9285-9526 being deleted. This clone was therefore used for cloning into the expression vector pJIT117 (Guerineau et al. 1988, Nucl. Acids Res. 16: 11380).

The cloning was carried out by isolation of the 771 bp SacI-HindIII fragment from pTAP3P and ligation into the SacI-HindIII-cleaved vector pJIT117. The clone, which comprises the promoter AP3P instead of the original promoter d35S, is called pJAP3P.

A DNA fragment which comprises the PIV2 intron of the gene ST-LS1 was prepared by means of PCR using plasmid DNA p35SGUS INT (Vancanneyt G. et al. (1990) Mol Gen Genet 220: 245-50) and the primer PR40 (Seq ID No. 30) and primer PR41 (Seq ID No. 31).

The PCR conditions were as follows:

The PCR for the amplification of the sequence of the intron PIV2 of the gene ST-LS1 was carried out in a 50 ∝l reaction batch, which comprised:
1 ml of p35SGUS INT
0.25 mM dNTPs
0.2 mMPR40 (SEQ ID No. 30)
0.2 mM PR41 (SEQ ID No. 31)
5 ml of 10×PCR buffer (TAKARA)
0.25 ml of R Taq polymerase (TAKARA)
28.8 ml of dist. water

The PCR was carried out under the following cycle conditions:
1×94° C. 2 minutes
35×94° C. 1 minute
53° C. 1 minute
72° C. 1 minute
1×72° C. 10 minutes

The PCR amplification using PR40 and PR41 resulted in a 206 bp fragment. Using standard methods, the amplificate was cloned into the PCR cloning vector pBluntII (Invitrogen) and the clone pBluntII-4041 was obtained. Sequencing of this clone using the primer SP6 confirmed a sequence which is identical with the corresponding sequence from the vector p35SGUS INT.

This clone was therefore used for the cloning into the vector pJAP3P (described above).

The cloning was carried out by isolation of the 206 bp SalI-BamHI fragment from pBluntII-40-41 and ligation using the SalI-BamHI-cleaved vector pJAP3P. The clone, which comprises the intron PIV2 of the gene ST-LS1 in the correct orientation connecting to the 3′ end of the rbcs transit peptide, is called pJAI1 and is suitable for preparing expression cassettes for the flower-specific expression of inverted-repeat transcripts.

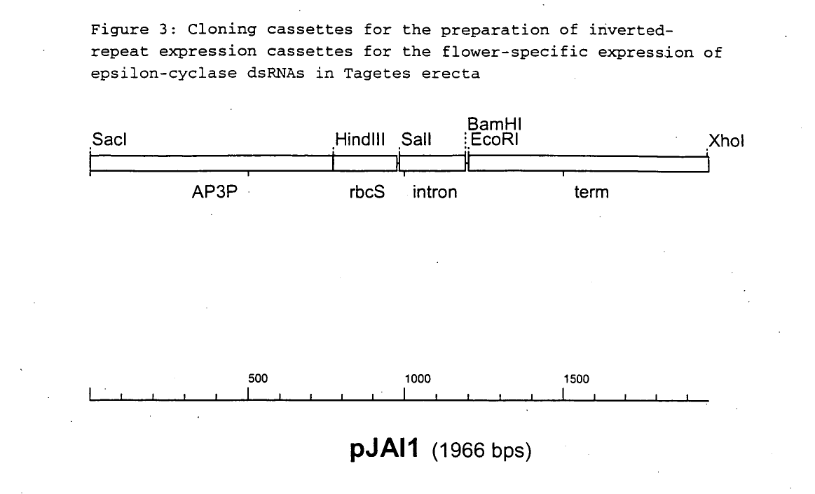

In FIG. 3, fragment AP3P contains the modified AP3P promoter (771 bp), fragment rbcs contains the rbcS transit peptide from pea (204 bp), fragment intron contains the intron PIV2 of the potato gene ST-LS1, and fragment term (761 bp) contains the polyadenylation signal of CaMV.

### EXAMPLE 3

### Preparation of Inverted-Repeat Expression Cassettes for the Flower-Specific Expression of Epsilon-Cyclase dsRNAs in Tagetes erecta (Directed Against the 5′ Region of the Epsilon-Cyclase cDNA)

The nucleic acid, which comprises the 5′ terminal 435 bp region of the epsilon-cyclase cDNA (Genbank accession no. AF251016), was amplified by means of polymerase chain reaction (PCR) from Tagetes erecta cDNA using a sense-specific primer (PR42 SEQ ID NO. 32) and an antisense-specific primer (PR43 SEQ ID NO. 33). The 5′ terminal 435 bp region of the epsilon-cyclase cDNA from Tagetes erecta is composed of 138 bp of 5′ nontranslated sequence (5′UTR) and 297 bp of the coding region corresponding to the N terminus.

For the preparation of total RNA from flowers of Tagetes, 100 mg of the frozen, pulverized flowers were transferred to a reaction vessel and taken up in 0.8 ml of Trizol buffer (LifeTechnologies). The suspension was extracted using 0.2 ml of chloroform. After centrifugation at 12 000 g for 15 minutes, the aqueous supernatant was removed, transferred to a new reaction vessel and extracted with one volume of ethanol. The RNA was precipitated using one volume of isopropanol, washed with 75% ethanol and the pellet was dissolved in DEPC water (overnight incubation of water with 1/1000 volume of diethyl pyrocarbonate at room temperature, subsequently autoclaved). The RNA concentration was determined photometrically. For the cDNA synthesis, 2.5 μg of total RNA were denatured for 10 min at 60° C., cooled on ice for 2 min and transcribed by means of a cDNA kit (Ready-to-go-you-prime-beads, Pharmacia Biotech) according to the manufacturer's instructions using an antisense-specific primer (PR17 SEQ ID NO. 29) in cDNA.

The conditions of the subsequent PCR reactions were as follows:

The PCR for the amplification of the PR42—PR43 DNA fragment, which comprises the 5′ terminal 435 bp region of the epsilon-cyclase, was carried out in a 50 ml reaction batch, which comprised:
1 ml of cDNA (prepared as described above)
0.25 mM dNTPs
0.2 mM PR42 (SEQ ID No. 32)
0.2 mM PR43 (SEQ ID No. 33)
5 ml of 10×PCR buffer (TAKARA)
0.25 ml of R Taq polymerase (TAKARA)
28.8 ml of dist. water

The PCR for the amplification of the PR44—PR45 DNA fragment, which comprises the 5′ terminal 435 bp region of the epsilon-cyclase, was carried out in a 50 ml reaction batch, which comprised:
1 ml of cDNA (prepared as described above)
0.25 mM dNTPs
0.2 mM PR44 (SEQ ID No. 34)
0.2 mM PR45 (SEQ ID No. 35)
5 ml of 10×PCR-buffer (TAKARA)
0.25 ml of R Taq Polymerase (TAKARA)
28.8 ml of dist. water

The PCR reactions were carried out under the following cycle conditions:
1×94° C. 2 minutes
35×94° C. 1 minute
58° C. 1 minute
72° C. 1 minute
1×72° C. 10 minutes

The PCR amplification using primers PR42 and PR43 resulted in a 443 bp fragment; PCR amplification using primers PR44 and PR45 resulted in a 444 bp fragment.

The two amplificates, the PR42—PR43 (HindIII-SalI sense) fragment and the PR44—PR45 (EcoRI-BamHI antisense) fragment were cloned into the PCR cloning vector pCR-BluntII (Invitrogen) using standard cloning methods. Sequencing using the primer SP6 in each case confirmed a sequence identical to the published sequence AF251016 (SEQ ID No. 7) apart from the restriction sites introduced. This clone was therefore used for the preparation of an inverted-repeat construct in the cloning vector pJAI1 (see example 2).

The first cloning step was carried out by isolation of the 444 bp PR44—PR45 BamHI-EcoRI fragment from the cloning vector pCR-BluntII (Invitrogen) and ligation with the BamHI-EcoRI-cleaved vector pJAI1. The clone, which comprises the 5′ terminal region of the epsilon-cyclase in the antisense orientation, is called pJAI2. As a result of the ligation, a transcriptional fusion results between the antisense fragment of the 5′ terminal region of the epsilon-cyclase and the poladenylation signal of CaMV.

The second cloning step was carried out by isolation of the 443 bp PR42—PR43 HindIII-SalI fragment from the cloning vector pCR-BluntII (Invitrogen) and ligation with the HindIII-SalI-cleaved vector pJAI2. The clone, which comprises the 435 bp 5′ terminal region of the epsilon-cyclase cDNA in the sense orientation, is called pJAI3. As a result of the ligation, a transcriptional fusion results between the AP3P and the sense fragment of the 5′ terminal region of the epsilon-cyclase.

For the preparation of an inverted-repeat expression cassette under the control of the CHRC promoter, a CHRC promoter fragment was amplified using genomic DNA from petunia (prepared according to standard methods) and the primers PRCHRC5 (SEQ ID No. 50) and PRCHRC3 (SEQ ID No. 51). The amplificate was cloned into the cloning vector pCR2.1 (Invitrogen). Sequencing of the resulting clone pCR2.1-CHRC using the primers M13 and T7 confirmed a sequence identical to the sequence AF099501. This clone was therefore used for cloning into the expression vector pJAI3.

The cloning was carried out by isolation of the 1537′ bp SacI-HindIII fragment from pCR2.1-CHRC and ligation into the SacI-HindIII-cleaved vector pJAI3. The clone, which comprises the promoter CHRC instead of the original promoter AP3P, is called pJCI3.

The preparation of the expression vectors for the Agrobacterium-mediated transformation of the AP3P or CHRC-controlled inverted-repeat transcripts in Tagetes erecta was carried out using the binary vector pSUN5 (WO02/00900).

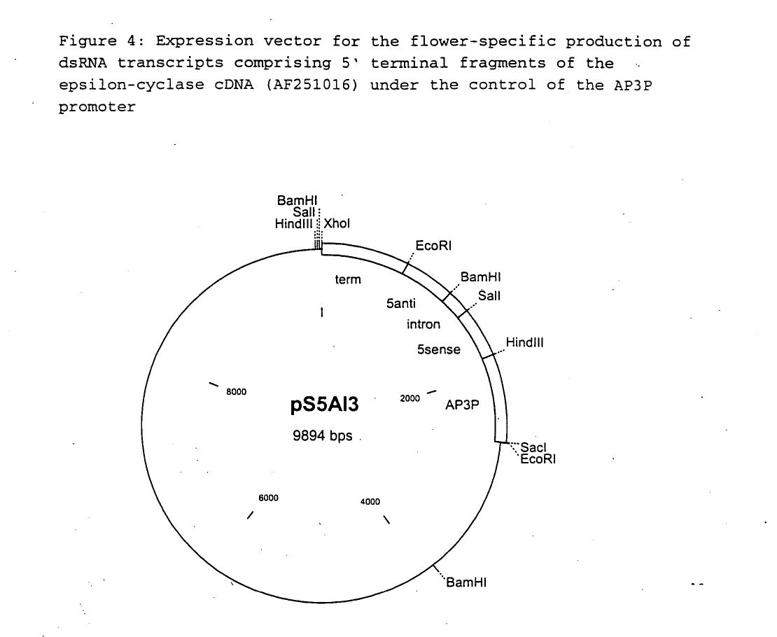

For the preparation of the expression vector pS5AI3, the 2622 bp SacI-XhoI fragment from pJAI3 was ligated using the SacI-XhoI-cleaved vector pSUN5 (FIG. 4, construct map).

In FIG. 4, fragment AP3P contains the modified AP3P promoter (771 bp), fragment 5sense contains the 5′ region of the epsilon-cyclase from Tagetes erecta (435 bp) in sense orientation, fragment intron contains the intron PIV2 of the potato gene ST-LS1, fragment 5anti contains the 5′ region of the epsilon-cyclase from Tagetes erecta (435 bp) in antisense orientation, and fragment term (761 bp) contains the poladenylation signal of CaMV.

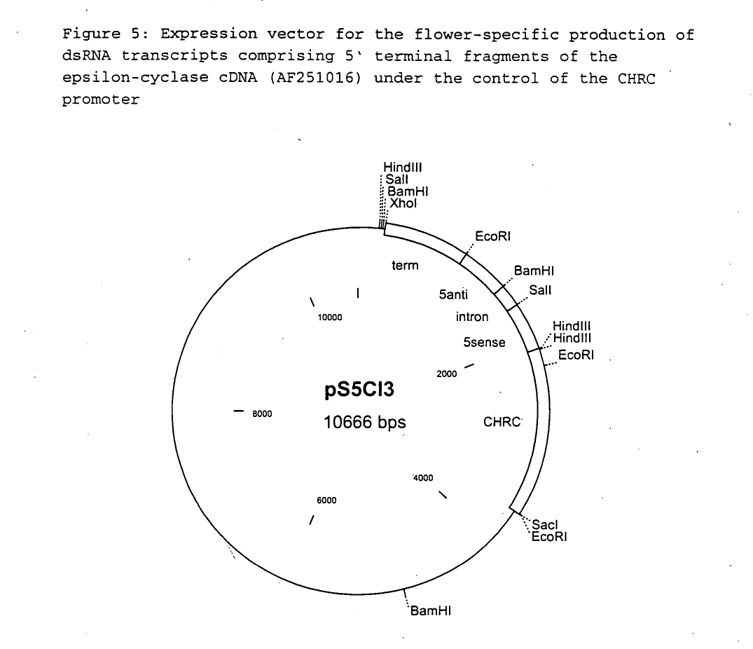

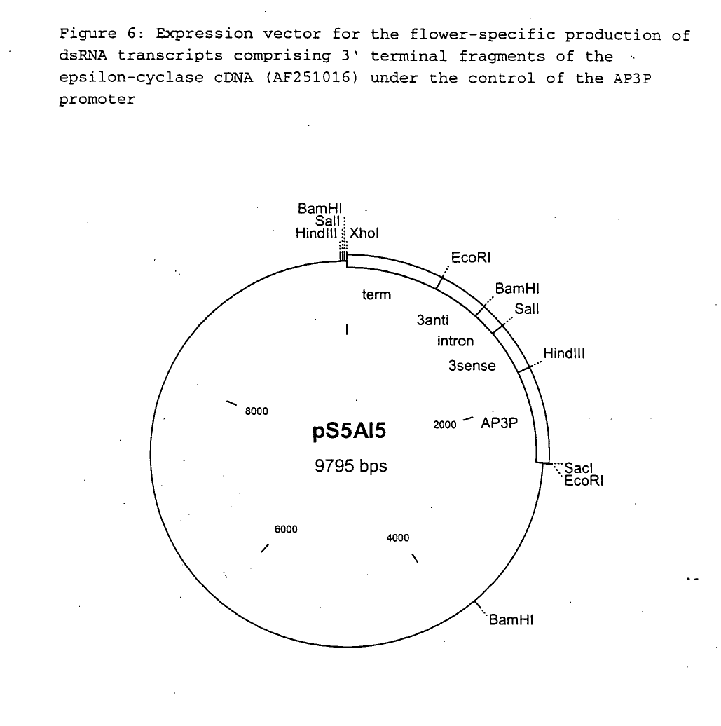

For the preparation of the expression vector pS5CI3, the 3394 bp SacI-XhoI fragment from pJCI3 was ligated with the SacI-XhoI-cleaved vector pSUN5 (FIG. 5, construct map).

In FIG. 5, fragment CHRC contains the promoter (1537 bp), fragment 5sense contains the 5′ region of the epsilon-cyclase from Tagetes erecta (435 bp) in sense orientation, fragment intron contains the intron PIV2 of the potato gene ST-LS1, fragment 5anti contains the 5′ region of the epsilon-cyclase from Tagetes erecta (435 bp) in antisense orientation, and fragment term (761 bp) contains the poladenylation signal of CaMV.

### EXAMPLE 4

### Preparation of an Inverted-Repeat Expression Cassette for the Flower-Specific Expression of Epsilon-Cyclase dsRNAs in Tagetes erecta (Directed Against the 3′ Region of the Epsilon-Cyclase cDNA)

The nucleic acid which comprises the 3′ terminal region (384 bp) of the epsilon-cyclase cDNA (Genbank accession no. AF251016) was amplified by means of polymerase chain reaction (PCR) from Tagetes erecta cDNA using a sense-specific primer (PR46 SEQ ID NO. 36) and an antisense-specific primer (PR47 SEQ ID NO. 37). The 3′ terminal region (384 bp) of the epsilon-cyclase cDNA from Tagetes erecta is composed of 140 bp of 3′-nontranslated sequence (3′UTR) and 244 bp of the coding region corresponding to the C terminus.

The preparation of total RNA from the flowers of Tagetes was carried out as described under example 3.

The cDNA synthesis was carried out as described under example 2 using the antisense-specific primer PR17 (SEQ ID No. 19).

The conditions of the subsequent PCR reactions were as follows:

The PCR for the amplification of the PR46—PR457 DNA fragment, which comprises the 3′ terminal 384 bp region of the epsilon-cyclase, was carried out in a 50 μl reaction batch, which comprised:
1 ml of cDNA (prepared as described above)
0.25 mM dNTPs
0.2 mM PR46 (SEQ ID No. 36)
0.2 mM PR47 (SEQ ID No. 37)
5 ml of 10×PCR-buffer (TAKARA)
0.25 ml of R Taq polymerase (TAKARA)
28.8 ml of dist. water

The PCR for the amplification of the PR48—PR49 DNA fragment, which comprises the 3′ terminal 384 bp region of the epsilon-cyclase, was carried out in a 50 μl reaction batch, which comprised:
1 ml of cDNA (prepared as described above)
0.25 mM dNTPs
0.2 mM PR48 (SEQ ID No. 38)
0.2 mM PR49—(SEQ ID No. 39)
5 ml of 10×PCR buffer (TAKARA)
0.25 ml of R Taq polymerase (TAKARA)
28.8 ml of dist. water

The PCR reactions were carried out under the following cycle conditions:
1×94° C. 2 minutes
35×94° C. 1 minute
58° C. 1 minute
72° C. 1 minute
1×72° C. 10 minutes

The PCR amplification using SEQ ID NO.36 and SEQ ID NO. 37 resulted in a 392 bp fragment; the PCR amplification using SEQ ID NO.38 and SEQ ID NO. 39 resulted in a 396 bp fragment.

The two amplificates, the PR46—PR47 fragment and the PR48—PR49 fragment, were cloned into the PCR cloning vector pCR-BluntII (Invitrogen) using standard methods. Sequencing using the primer SP6 in each case confirmed a sequence identical to the published sequence AF251016 (SEQ ID NO. 7) apart from the restriction sites introduced. This cloning was therefore used for the preparation of an inverted-repeat construct in the cloning vector pJAI1 (see example 2).

The first cloning step was carried out by isolation of the 396 bp PR48—PR49 BamHI-EcoRI fragment from the cloning vector pCR-BluntII (Invitrogen) and ligation with the BamHI-EcoRI-cleaved vector pJAI1. The clone, which comprises the 3′ terminal region of the epsilon-cyclase in the antisense orientation, is called pJAI4. As a result of the ligation, a transcriptional fusion results between the antisense fragment of the 3′ terminal region of the epsilon-cyclase and the polyadenylation signal of CaMV.

The second cloning step was carried out by isolation of the 392 bp PR46—PR47 HindIII-SalI fragment from the cloning vector pCR-BluntII (Invitrogen) and ligation with the HindIII-SalI-cleaved vector pJAI4. The clone, which comprises the 392 bp 3′ terminal region of the epsilon-cyclase cDNA in the sense orientation, is called pJAI5. As a result of the ligation, a transcriptional fusion results between the AP3P and the sense fragment 3′ terminal region of the epsilon-cyclase.

The preparation of an expression vector for the Agrobacterium-mediated transformation of the AP3P-controlled inverted-repeat transcript in Tagetes erecta was carried out using the binary vector pSUN5 (WO02/00900). For the preparation of the expression vector pS5AI5, the 2523 bp SacI-XhoI fragment from pJAI5 was ligated with the SacI-XhoI-cleaved vector pSUN5 (FIG. 5, construct map).

In FIG. 5, fragment AP3P contains the modified AP3P promoter (771 bp), fragment sense contains the 3′ region of the epsilon-cyclase from Tagetes erecta (435 bp) in sense orientation, fragment intron contains the intron IV2 of the potato gene ST-LS1, fragment anti contains the 3′ region of the epsilon cyclase from Tagetes erecta (435 bp) in antisense orientation, and fragment term (761 bp) contains the polyadenylation signal of CaMV.

### EXAMPLE 5

### Cloning of the Epsilon-Cyclase Promoter

A 199 bp fragment or the 312 bp fragment of the epsilon-cyclase promoter was isolated by two independent cloning strategies, inverse PCR (adapted from Long et al. Proc. Natl. Acad. Sci USA 90: 10370) and TAIL PCR (Liu Y-G. et al. (1995) Plant J. 8: 457-463) using genomic DNA (isolated according to a standard method from Tagetes erecta, line Orangenprinz).

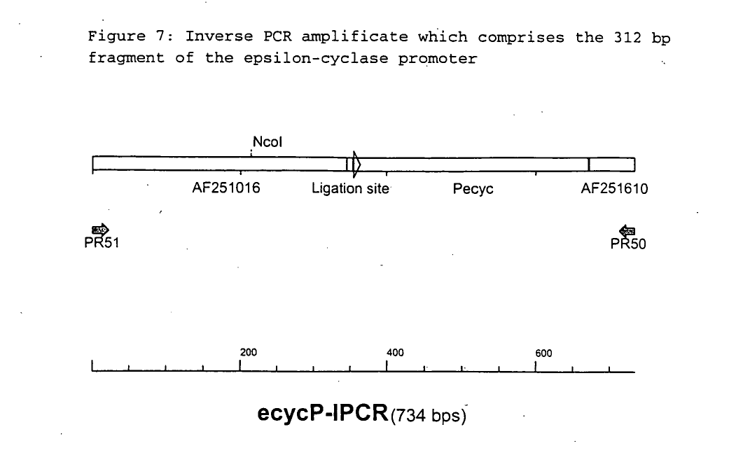

For the inverse PCR batch, 2 μg of genomic DNA were digested in a 25 μl reaction batch using EcoRV and RsaI, subsequently diluted to 300 μl and and religated overnight at 16° C. with 3 U of ligase. Using the primers PR50 (SEQ ID NO. 40) and PR51 (SEQ ID NO. 41), a fragment was prepared by PCR amplification which, in each case in sense orientation, ligates 354 bp of the epsilon-cyclase cDNA (Genbank Accession AF251016) to 300 bp of the epsilon-cyclase promoter, and 70 bp of the 5′ terminal region which comprises cDNA epsilon-cyclase (see FIG. 7).

The conditions of the PCR reactions were as below:

The PCR for the amplification of the PR50—PR51 DNA fragment which, inter alia, comprises the 312 bp promoter fragment of the epsilon-cyclase, was carried out in a 50 μl reaction batch, which comprised:
1 ml of ligation batch (prepared as described above)
0.25 mM dNTPs
0.2 mM PR50 (SEQ ID No. 40)
0.2 mM PR51 (SEQ ID No. 41)
5 ml of 10×PCR buffer (TAKARA)
0.25 ml of R Taq polymerase (TAKARA)
28.8 ml of dist. water

The PCR reactions were carried out under the following cycle conditions:
1×94° C. 2 minutes
35×94° C. 1 minute
53° C. 1 minute
72° C. 1 minute
1×72° C. 10 minutes

The PCR amplification using primers PR50 and PR51 resulted in a 734 bp fragment, which, inter alia, comprises the 312 bp promoter fragment of the epsilon-cyclase (FIG. 7).

The amplificate was cloned into the PCR cloning vector pCR2.1 (Invitrogen) using standard methods. Sequencing using the primers M13 and T7 afforded the sequence SEQ ID No. 9. This sequence was reproduced in an independent amplification experiment and thus represents the nucleotide sequence in the Tagetes erecta line Orangenprinz used.

For the TAIL PCR batch, three successive PCR reactions were carried out, in each case with different gene-specific primers (nested primers).

The TAIL1 PCR was carried out in a 20 ml reaction batch, which comprised:
1 ng of genomic DNA (prepared as described above)
0.2 mM of each dNTP
0.2 mM PR60 (SEQ ID No. 42)
0.2 mM AD1 (SEQ ID No. 45)
2 ml of 10×PCR buffer (TAKARA)
0.5 ml of R Taq polymerase (TAKARA)
made up to 20 μl with dist. water

AD1 here initially represents a mixture of primers of the sequences (a/c/g/t)tcga(g/c)t(a/t)t(g/c)g(a/t)gtt.

The PCR reaction TAIL1 was carried out under the following cycle conditions
1×93° C.: 1 min., 95° C.: 1 min.
5×94° C.: 30 sec., 62° C.: 1 min., 72° C.: 2.5 min.
1×94° C.: 30 sec., 25° C.: 3 min., gradient to 72° C. in 3 min.
72° C.: 2.5 min
15×94° C.: 10 sec., 68° C.: 1-min., 72° C.: 2.5 min.;
94° C.: 10 sec., 68° C.: 1 min., 72° C.: 2.5 min.;
94° C.: 10 sec., 29° C.: 1 min., 72° C.: 2.5 min.
1×72° C.: 5 min.

The TAIL2 PCR was carried out in a 21 μl reaction batch, which comprised:
1 μl of a 1:50 dilution of the TAIL1 reaction batch (prepared as described above)
0.8 mM dNTP
0.2 mM PR61 (SEQ ID No. 43)
0.2 mM AD1 (SEQ ID No. 45)
2 μl of 10×PCR buffer (TAKARA)
0.5 μl of R Taq polymerase (TAKARA)
made up to 21 μl with dist. water

The PCR reaction TAIL2 was carried out under the following cycle conditions:
12×94° C.: 10 seconds, 64° C.: 1 minute, 72° C.: 2.5 minutes;
94° C.: 10 seconds, 64° C.: 1 minute, 72° C.: 2.5 minutes;
94° C.: 10 seconds, 29° C.: 1 minute, 72° C.: 2.5 minutes
1×72° C.: 5 minutes

The TAIL3 PCR was carried out in a 100 ml reaction batch, in which was contained:
1 μl of a 1:10 dilution of the TAIL2 reaction batch (prepared as described above)
0.8 mM dNTP
0.2 mM PR63 (SEQ ID No. 44)
0.2 mM AD1 (SEQ ID No. 45)
10 μl of 10×PCR buffer (TAKARA)
0.5 μl of R Taq polymerase (TAKARA)
made up to 100 μl with dist. water

The PCR reaction TAIL3 was carried out under the following cycle conditions:
20×94° C.: 15 seconds, 29° C.: 30 seconds, 72° C.: 2 minutes
1×72° C.: 5 minutes

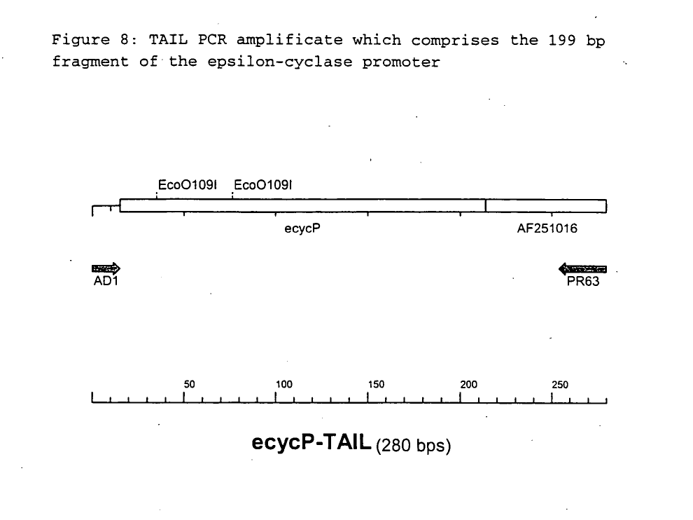

The PCR amplification using primers PR63 and AD1 resulted in a 280 bp fragment which, inter alia, comprises the 199 bp promoter fragment of the epsilon-cyclase (FIG. 8).

The amplificate was cloned into the PCR cloning vector pCR2.1 (Invitrogen) using standard methods. Sequencing using the primers M13 and T7 afforded the sequence SEQ ID No. 9. This sequence is identical with the ε-cyclase region within the sequence SEQ ID No. 7, which was isolated using the IPCR strategy, and thus represents the nucleotide sequence in the Tagetes erecta line Orangenprinz used.

The pCR2.1 clone, which comprises the 312 bp fragment (SEQ ID No. 9) of the epsilon-cyclase promoter, which was isolated by the IPCR strategy, is called pTA-ecycP and was used for the preparation of the IR constructs.

### EXAMPLE 6

### Preparation of an Inverted-Repeat Expression Cassette for the Flower-Specific Expression of Epsilon-Cyclase dsRNAs in Tagetes erecta (Directed Against the Promoter Region of the Epsilon-Cyclase cDNA)

The expression of inverted-repeat transcripts consisting of promoter fragments of the epsilon-cyclase in Tagetes erecta was carried out under the control of a modified version AP3P of the flower-specific promoter AP3 from Arabidopsis (see example 2) or of the flower-specific promoter CHRC (Genbank accession no. AF099501). The inverted-repeat transcript in each case contains an epsilon-cyclase promoter fragment in correct orientation (sense fragment) and an identical sequence epsilon-cyclase promoter fragment in opposite orientation (antisense fragment), which are connected to one another by a functional intron (see example 2).

The promoter fragments were prepared by means of PCR using plasmid DNA (clone pTA-ecycP, see example 5) and the primers PR124 (SEQ ID No. 46) and PR126 (SEQ ID No. 48) or the primers PR125 (SEQ ID No. 47) and PR127 (SEQ ID No. 49).

The conditions of the PCR reactions were as below:

The PCR for the amplification of the PR124-PR126 DNA fragment which comprises the promoter fragment of the epsilon-cyclase was carried out in a 50 ml reaction batch, which comprised:
1 ml of cDNA (prepared as described above)
0.25 mM dNTPs
0.2 mM PR124 (SEQ ID No. 46)
0.2 mM PR126 (SEQ ID No. 48)
5 ml of 10×PCR buffer (TAKARA)
0.25 ml of R Taq polymerase (TAKARA)
28.8 ml of dist. water

The PCR for the amplification of the PR125-PR127 DNA fragment, which comprises the 312 bp promoter fragment of the epsilon-cyclase, was carried out in a 50 μl reaction batch, which comprised:
1 μl of cDNA (prepared as described above)
0.25 mM dNTPs
0.2 mM PR125 (SEQ ID No. 47)
0.2 mM PR127 (SEQ ID No. 49)
5 μl of 10×PCR buffer (TAKARA)
0.25 μl of R Taq polymerase (TAKARA)
28.8 μl of dist. water

The PCR reactions were carried out under the following cycle conditions:
1×94° C. 2 minutes
35×94° C. 1 minute
53° C. 1 minute
72° C. 1 minute
1×72° C. 10 minutes

The PCR amplification using primers PR124 and PR126 resulted in a 358 bp fragment; the PCR amplification using primers PR125 and PR127 resulted in a 361 bp fragment.

The two amplificates, the PR124-PR126 (HindIII-SalI sense) fragment and the PR125-PR127 (EcoRI-BamHI antisense) fragment, were cloned into the PCR cloning vector pCR-BluntII (Invitrogen) using standard methods. Sequencing using the primer SP6 in each case confirmed a sequence which, apart from the restriction sites introduced, is identical to SEQ ID No. 7. These clones were therefore used for the preparation of an inverted-repeat construct in the cloning vector pJAI1 (see example 2).

The first cloning step was carried out by isolation of the 358 bp PR124-PR126 HindIII-SalI fragment from the cloning vector pCR-BluntII (Invitrogen) and ligation with the BamHI-EcoRI-cleaved vector pJAI1. The clone, which comprises epsilon-cyclase promoter fragment in the sense orientation, is called cs43. As a result of the ligation, the sense fragment of the epsilon-cyclase promoter is inserted between the AP3P promoter and the intron.

The second cloning step was carried out by isolation of the 361 bp PR125-PR127 BamHI-EcoRI fragment from the cloning vector pCR-BluntII (Invitrogen) and ligation with BamHI-EcoRI-cleaved vector cs43. The clone, which comprises the epsilon-cyclase promoter fragment in the antisense orientation, is called cs44. As a result of the ligation, a transcriptional fusion results between the intron and the antisense fragment of the epsilon-cyclase promoter.

For the preparation of an inverted-repeat expression cassette under the control of the CHRC promoter, a CHRC promoter fragment was amplified using genomic DNA from petunia (prepared according to standard methods) and the primers PRCHRC3′ (SEQ ID NO. 51) and PRCHRC5′ (SEQ ID NO. 50). The amplificate was cloned into the cloning vector pCR2.1 (Invitrogen). Sequencing of the resulting clone pCR2.1-CHRC using the primers M13 and T7 confirmed a sequence identical to the sequence AF099501. This clone was therefore used for cloning into the expression vector cs44.

The cloning was carried out by isolation of the 1537 bp SacI-HindIII fragment from pCR2.1-CHRC and ligation into the SacI-HindIII-cleaved vector cs44. The clone, which comprises the promoter CHRC instead of the original promoter AP3P, is called cs45.

For the preparation of an inverted-repeat expression cassette under the control of two promoters, the CHRC promoter and the AP3P promoter, the AP3P promoter was cloned in antisense orientation onto the 3′ terminus of the epsilon-cyclase antisense fragment in cs45. The AP3P promoter fragment from pJAI1 was amplified using the primers PR128 and PR129. The amplificate was cloned into the cloning vector pCR2.1 (Invitrogen). This clone pCR2.1-AP3PSX was used for the preparation of an inverted-repeat expression cassette under the control of two promoters.

The cloning was carried out by isolation of the 771 bp SalI-XhoI fragment from pCR2.1-AP3PSX and ligation into the XhoI-cleaved vector cs45. The clone, which on the 3′ side of the inverted repeat comprises the promoter AP3P in antisense orientation, is called cs46.

The preparation of the expression vectors for the Agrobacterium-mediated transformation of the AP3P-controlled inverted-repeat transcript in Tagetes erecta was carried out using the binary vector pSUN5 (WO02/00900).

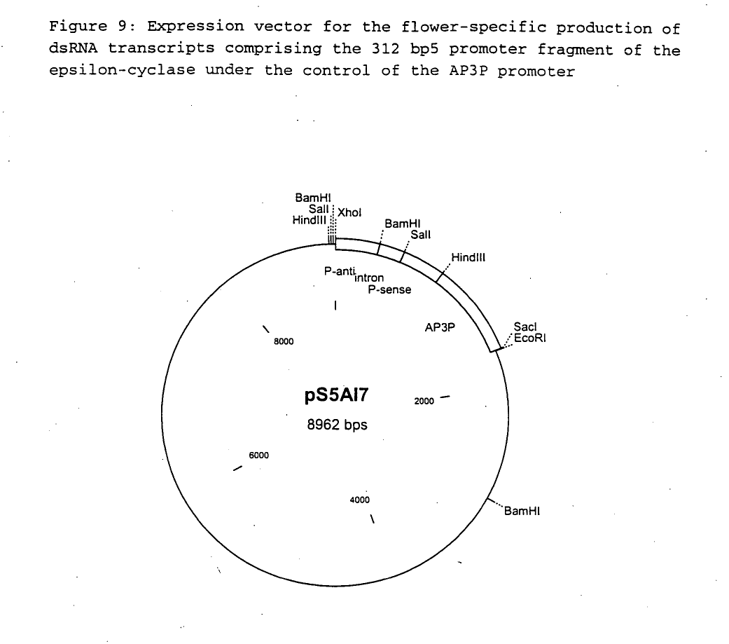

For the preparation of the expression vector pS5AI7, the 1685 bp SacI-XhoI fragment from cs44 was ligated with the SacI-XhoI-cleaved vector pSUN5 (FIG. 9, construct map).

In FIG. 9, fragment AP3P contains the modified AP3P promoter (771 bp), fragment P-sense contains the 312 bp promoter fragment of the epsilon-cyclase in sense orientation, fragment intron contains the intron IV2 of the potato gene ST-LS1), and fragment P-anti contains the 312 bp promoter fragment of the epsilon-cyclase in antisense orientation.

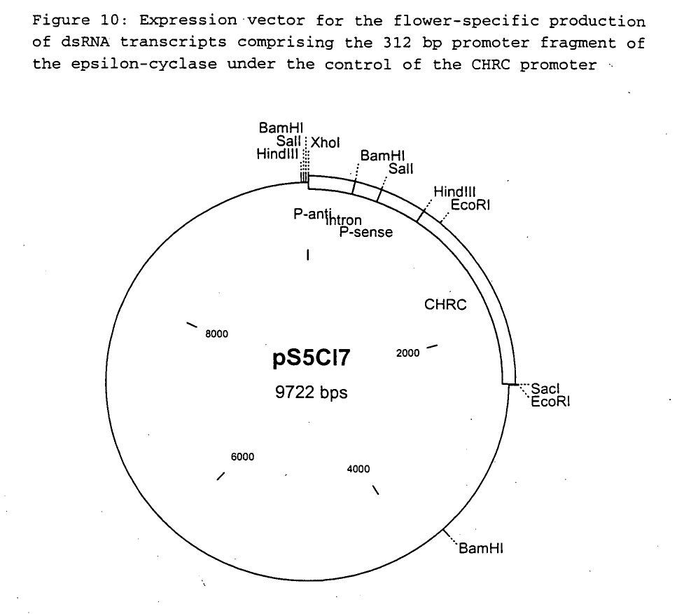

For the preparation of the expression vector pS5CI7, the 2445 bp SacI-XhoI fragment from cs45 was ligated with the SacI-XhoI-cleaved vector pSUN5 (FIG. 10, construct map).

In FIG. 10, fragment CHRC contains the CHRC promoter (1537 bp), fragment P-sense contains the 312 bp promoter fragment of the epsilon-cyclase in sense orientation, fragment intron contains the intron IV2 of the potato gene ST-LS1), and fragment P-anti contains the 312 bp promoter fragment of the epsilon-cyclase in antisense orientation.

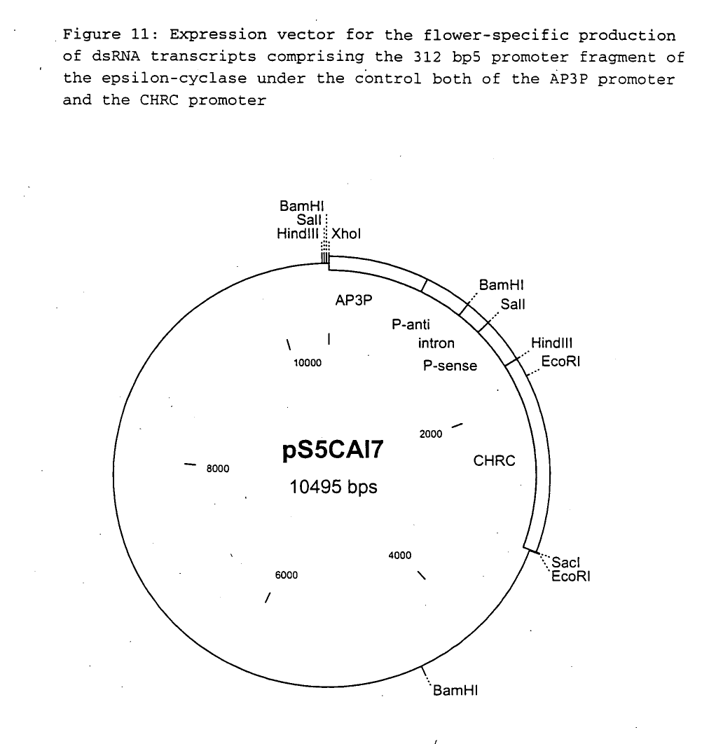

For the preparation of the expression vector pS5CI7, the 3219 bp SacI-XhoI fragment from cs46 was ligated with the SacI-XhoI-cleaved vector pSUN5 (FIG. 11, construct map).

In FIG. 11, fragment CHRC contains the CHRC promoter (1537 bp), fragment P-sense contains the 312 bp promoter fragment of the epsilon-cyclase in sense orientation, fragment intron contains the intron IV2 of the potato gene ST-LS1), fragment P-anti contains the 312 bp promoter fragment of the epsilon-cyclase in antisense orientation and the fragment AP3P contains the 771 bp AP3P promoter fragment in antisense orientation.

### EXAMPLE 7

### Production of Transgenic Tagetes Plants

Tagetes seeds are sterilized and laid on germination medium (MS medium; Murashige and Skoog, Physiol. Plant. 15 (1962), 473-497) pH 5.8, 2% sucrose). Germination is carried out in a temperature/light/time interval of 18 to 28° C./20 to 200 ∝E/3 to 16 weeks, but preferably at 21° C., 20 to 70 ∝E, for 4 to 8 weeks.

All leaves of the by then developed in vitro plants are harvested and cut diagonally to the middle rib. The leaf explants resulting thereby having a size of 10 to 60 mm² are stored in the course of the preparation in liquid MS medium at room temperature for at most 2 h.

The Agrobacterium tumefaciens strain EHA105 was transformed using the binary plasmid PS5AI3. The transformed A. tumefaciens strain EHA105 was grown overnight under the following conditions: a single colony was inoculated into YEB (0.1% yeast extract, 0.5% beef extract, 0.5% peptone, 0.5% sucrose, 0.5% magnesium sulfate×7H₂O) with 25 mg/l of kanamycin and grown at 28° C. for 16 to 20 h. Subsequently, the bacterial suspension was harvested by centrifugation at 6000 g for 10 min and resuspended in liquid MS medium in such a way that an OD₆₀₀ of about 0.1 to 0.8 resulted. This suspension was used for the coculturing with the leaf material.

Immediately before the coculturing, the MS medium in which the leaves have been stored is replaced by the bacterial suspension. The leaves were incubated in the Agrobacteria suspension for 30 min with gentle shaking at room temperature. Subsequently, the infected explants are laid on an MS medium solidified with agar (e.g. 0.8% plant agar (Duchefa, NL) containing growth regulators, such as, for example, 3 mg/l of benzylaminopurine (BAP) and 1 mg/l of indolylacetic acid (IAA). The orientation of the leaves on the medium is unimportant. The explants are cultured for 1 to 8 days, but preferably for 6 days; the following conditions can be used here: light intensity: 30 to 80 ∝mol/m²×sec, temperature: 22 to 24° C., light/dark change of 16/8 hours. Subsequently, the cocultured explants are transferred to fresh MS medium, preferably with the same growth regulators, this second medium additionally comprising an antibiotic for suppressing the bacterial growth. Timentin in a concentration of 200 to 500 mg/l is very suitable for this purpose. The second selective component employed is one for the selection of the transformation success. Phosphinothricin in a concentration of 1 to 5 mg/l selects very efficiently, but other selective components are also conceivable according to the process to be used.

After one to three weeks in each case, the explants are transferred to fresh medium until sprout buds and small sprouts develop, which are then transferred to the same basal medium including timentin and PPT or alternative components containing growth regulators, namely, for example, 0.5 mg/l of indolylbutyric acid (IBA) and 0.5 mg/l of gibberellic acid GA₃, for rooting. Rooted sprouts can be transferred to a greenhouse.

Additionally to the method described, the following advantageous modifications are possible:
Before the explants are infected with the bacteria, they can be preincubated for 1 to 12 days, preferably 3 to 4, on the medium described above for the coculture. Subsequently, the infection, coculture and selective regeneration are carried out as described above.
The pH for the regeneration (normally 5.8) can be lowered to pH 5.2. The control of the growth of the Agrobacteria is thereby improved.
The addition of AgNO₃ (3 to 10 mg/l) to the regeneration medium improves the condition of the culture including the regeneration itself.
Components which reduce the phenol formation and are known to the person skilled in the art, such as, for example, citric acid, ascorbic acid, PVP and many others, have a positive effect on the culture.
Liquid culture medium can also be used for the entire process. The culture can also be incubated on commercially available supports, which are positioned on the liquid medium.

According to the transformation method described above, the following lines were obtained with the following expression constructs:

With p76Bgene (from Example 1) was obtained: MK14-1-1 With pS5AI3 was obtained: CS30-1, CS30-3 and CS30-4

### EXAMPLE 8

### Characterization of the Transgenic Plants

### Example 8.1

### CS30-1, CS30-3 and CS30-4

The flower material of the transgenic Tagetes erecta plants CS30-1, CS30-3 and CS30-4 from example 7 was ground in liquid nitrogen in a mortar and the powder (approximately 250 to 500 mg) was extracted with 100% acetone (three times 500 μl each). The solvent was evaporated and the carotenoids were resuspended in 100 μl of acetone.

By means of a C30 reverse phase column, it was possible to differentiate between mono- and diesters of the carotenoids. HPLC running conditions were almost identical to a published method (Frazer et al. (2000), Plant Journal 24(4): 551-558). Identification of the carotenoids was possible on the basis of the UV-VIS spectra.

Table 1 shows the carotenoid profile in Tagetes petals of the transgenic Tagetes and control Tagetes plants produced according to the examples described above. All carotenoid amounts are indicated in [μg/g] of fresh weight; percentage changes compared with the control plants are indicated in brackets.

In comparison to the genetically unmodified control plant, the genetically modified plants have a markedly increased content of carotenoids of the “β-carotene pathway”, such as, for example, β-carotene and zeaxanthin, and a markedly reduced content of carotenoids of the “α-carotene pathway”, such as, for example, lutein.

TABLE 1

| Plant | Lutein | β-Carotene | Zeaxanthin | Violaxanthin | Total carotenoids |
|---|---|---|---|---|---|
| Control | 260 | 4.8 | 2.7 | 36 | 304 |
| CS 30-1 | 35 (−86%) | 13 (+170%) | 4.4 (+62%) | 59 (+63%) | 111 (−63%) |
| Control | 456 | 6.4 | 6.9 | 58 | 527 |
| CS 30-3 | 62 (−86%) | 13 (+103%) | 8.9 (+29%) | 75 (+29%) | 159 (−70%) |
| CS 30-4 | 68 (−85%) | 9.1 (+42%) | 5.7 (−17%) | 61 (+5%) | 144 (−73%) |
| Control | 280 | 4.1 | 2.6 | 42 | 329 |
| CS 32-9 | 69 (−75%) | 5.5 (+34%) | 2.3 (−12%) | 25 (−38%) | 102 (−69%) |

### Example 8.2

### Reduction of the ε-Cyclase Activity in Tagetes erecta by Antisense CS 32-9

Using conventional methods known to the person skilled in the art, a Tagetes erecta antisense line CS32-9 was produced as a comparison example in which the reduction of the ε-cyclase activity took place by means of antisense. The carotenoid profile of this line (CS32-9), measured according to the method described above is likewise shown in table 1.

### Example 8.3

### Alkaline Hydrolysis of Carotenoid Esters and Identification of the Carotenoids of MK14-1-1

The flower leaves of the transgenic Tagetes erecta plants MK14-1-1 from example 7 were ground in liquid nitrogen in a mortar and the petal powder (approximately 20 mg) was extracted with 100% acetone (three times 500 μl each). The solvent was evaporated and the residue was taken up in 180 μl of acetone. In order to guarantee homogeneity of the extract, the extract was treated with ultrasound for two minutes.

20 μl of 10% strength KOH in methanol were added to the extract and it was shaken for 30 min at room temperature at 1000-1300 rpm. After this, the extract was titrated with HCl to pH 7.5 and centrifuged at 10000 g for 10 min.

The supernatant was analyzed by means of a C30 reverse phase column. HPLC running conditions were almost identical to a published method (Frazer et al. (2000), Plant Journal 24(4): 551-558). Identification of the carotenoids was possible on the basis of the UV-VIS spectra and on the basis of the masses.

The overexpression of the β-cyclase (Bgene) according to the invention from Lycopersicon esculentum under the control of the flower-specific promoter P76 from Arabidopsis thaliana in Tagetes erecta surprisingly led not to the accumulation of greater amounts of β-carotenoids but to a drastic lowering of the amount of α-carotenoids in favour of the amount of β-carotenoids.

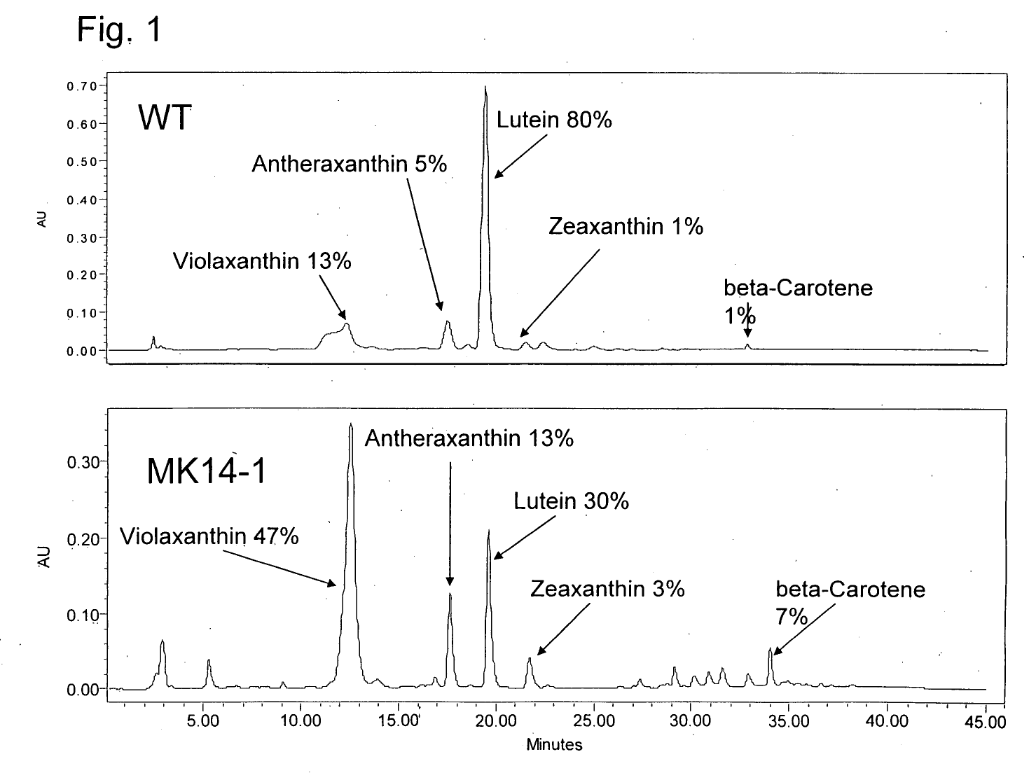

By this means, the amounts of α-carotenoid present in the flower of Tagetes erecta were reduced from over 80% to below 30% of the total carotenoids in the wild-type and the proportion of the β-carotenoids in the total carotenoid content increased from below 20% to over 70% in the wild-type (see FIG. 1).

1. A process for the production of β-carotenoids by culturing genetically modified plants which, in comparison to the wild-type, have an increased β-cyclase activity in plant tissues comprising photosynthetically inactive plastids, and the increased β-cyclase activity is caused by a β-cyclase comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, with the proviso that tomato is excluded as a plant.

2. A process as claimed in claim 1, wherein, for the increase in the β-cyclase activity the gene expression of a nucleic acid encoding a β-cyclase comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, is increased compared to the wild-type.

3. A process as claimed in claim 2, wherein, for the increase in the gene expression, nucleic acids are introduced into the plants which encode β-cyclases comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2.

4. A process as claimed in claim 3, wherein nucleic acids comprising the sequence SEQ. ID. NO. 1 are introduced.

5. A process as claimed in claim 2, wherein the expression of the β-cyclase takes place under the control of a promoter which guarantees the expression of the β-cyclase in the plant tissues comprising photosynthetically inactive plastids.

6. A process as claimed in claim 1, wherein the genetically modified plants are used which have the highest expression rate of the β-cyclase in plant tissues comprising photosynthetically inactive plastids.

7. A process as claimed in claim 6, wherein the expression of the β-cyclase takes place under the control of a promoter specific for the plant tissue.

8. A process as claimed in claim 1, wherein the plants additionally have an increased hydroxylase activity, compared to the wild-type.

9. A process as claimed in claim 8, wherein, for the additional increase in the hydroxylase activity, the gene expression of a nucleic acid encoding a hydroxylase is increased compared to the wild-type.

10. A process as claimed in claim 9, wherein, for the increase in the gene expression, a nucleic acid encoding a hydroxylase is introduced into the plant.

11. A process as claimed in claim 10, wherein, as a nucleic acid encoding a hydroxylase, nucleic acids are introduced which encode a hydroxylase comprising the amino acid sequence SEQ ID NO: 9 or a sequence derived from this sequence by substitution, insertion or deletion of amino acids, which have an identity of at least 20% at the amino acid level with the sequence SEQ ID NO: 9.

12. A process as claimed in claim 11, wherein nucleic acids comprising the sequence SEQ ID NO: 8 are introduced.

13. A process as claimed in claim 1, wherein the genetically modified plants are used which have the highest expression rate of the hydroxylase in plant tissues comprising photosynthetically inactive plastids.

14. A process as claimed in claim 13, wherein the expression of the hydroxylase takes place under the control of a promoter specific for the plant tissue.

15. A process as claimed in claim 1, wherein the plants, compared to the wild-type, additionally have a reduced activity of at least one of the activities selected from the group consisting of ε-cyclase activity and endogenous β-hydroxylase activity.

16. A process as claimed in claim 15, wherein the reduction of the ε-cyclase activity and/or of the endogenous β-hydroxylase activity in plants is achieved by at least one of the following processes:
a) introduction of at least one double-stranded ε-cyclase ribonucleic acid sequence and/or endogenous β-hydroxylase ribonucleic acid sequence or an expression cassette or expression cassettes guaranteeing their expression in plants,
b) introduction of at least one ε-cyclase antisense ribonucleic acid sequence and/or endogenous β-hydroxylase antisense ribonucleic acid sequence or an expression cassette or expression cassettes guaranteeing their expression in plants,
c) introduction of at least one ε-cyclase antisense ribonucleic acid sequence and/or endogenous β-hydroxylase antisense ribonucleic acid sequence in each case combined with a ribozyme or an expression cassette or expression cassettes guaranteeing their expression in plants,
d) introduction of at least one ε-cyclase sense ribonucleic acid sequence and/or endogenous β-hydroxylase sense ribonucleic acid sequence for the induction of a cosuppression or of an expression cassette or expression cassettes guaranteeing their expression in plants,
e) introduction of at least one DNA- or protein-binding factor against an ε-cyclase gene, RNA or protein and/or endogenous β-hydroxylase gene, RNA or protein or an expression cassette or expression cassettes guaranteeing its expression in plants,
f) introduction of at least one viral nucleic acid sequence or nucleic acid sequences bringing about the ε-cyclase RNA and/or endogenous β-hydroxylase RNA degradation or an expression cassette or expression cassettes guaranteeing their expression in plants,
g) introduction of at least one construct for the production of an insertion, deletion, inversion or mutation in an ε-cyclase gene and/or endogenous β-hydroxylase gene in plants.

17. A process as claimed in claim 16, embodiment a), wherein an RNA is introduced into the plant, which has a region having double-strand structure and in this region comprises a nucleic acid sequence which
a) is identical with at least one part of the plant-intrinsic ε-cyclase transcript and/or
b) is identical with at least one part of the plant-intrinsic ε-cyclase promoter sequence.

18. A process as claimed in claim 17, wherein the region having double-strand structure comprises a nucleic acid sequence which is identical with at least one part of the plant-intrinsic ε-cyclase transcript and comprises the 5′ end or the 3′ end of the plant-intrinsic nucleic acid encoding an ε-cyclase.

19. A process as claimed in claim 16, embodiment a), wherein an RNA is introduced into the plant which has a region having double-strand structure and in this region comprises a nucleic acid sequence which
a) is identical with at least one part of the plant-intrinsic, endogenous β-hydroxylase transcript and/or
b) is identical with at least one part of the plant-intrinsic, endogenous β-hydroxylase promoter sequence.

20. A process as claimed in claim 19, wherein the region having double-strand structure contains a nucleic acid sequence which is identical with at least one part of the plant-intrinsic, endogenous β-hydroxylase transcript and comprises the 5′ end or the 3′ end of the plant-intrinsic nucleic acid encoding an endogenous β-hydroxylase.

21. A process as claimed in claim 15, wherein the genetically modified plants are used which have the lowest expression rate of an ε-cyclase and/or endogenous β-hydroxylase in plant tissues comprising photosynthetically inactive plastids.

22. A process as claimed in claim 16, wherein the transcription of the double-stranded ribonucleic acid sequence as set forth in claim 16, embodiment a) and/or the antisense sequences as set forth in claim 16, embodiment b) takes place under the control of a promoter which is specific for the plant tissues comprising photosynthetically inactive plastids.

23. A process as claimed in claim 1, wherein, after culturing, the genetically modified plants are harvested and subsequently the β-carotenoids are isolated from the plants or the plant tissues comprising photosynthetically inactive plastids.

24. A process as claimed in claim 1, wherein the plant tissue comprising photosynthetically inactive plastids is selected from the group consisting of flower, fruit and tuber.

25. A process as claimed in claim 24, wherein the genetically modified plant used which, in comparison to the wild-type, has an increased β-cyclase activity in flowers, is a plant selected from the families Ranunculaceae, Berberidaceae, Papaveraceae, Cannabaceae, Rosaceae, Fabaceae, Linaceae, Vitaceae, Brassiceae, Cucurbitaceae, Primulaceae, Caryophyllaceae, Amaranthaceae, Gentianaceae, Geraniaceae, Caprifoliaceae, Oleaceae, Tropaeolaceae, Solanaceae, Scrophulariaceae, Asteraceae, Liliaceae, Amaryllidaceae, Poaceae, Orchidaceae, Malvaceae, Illiaceae or Lamiaceae.

26. A process as claimed in claim 25, wherein the plant used is a plant selected from the plant genera Marigold, Tagetes erecta, Tagetes patula, Acacia, Aconitum, Adonis, Arnica, Aqulegia, Aster, Astragalus, Bignonia, Calendula, Calendula officinalis, Caltha, Campanula, Canna, Centaurea, Cheiranthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hisbiscus, Heliopsis, Hypericum, Hypochoeris, Impatiens, Iris, Jacaranda, Kerria, Laburnum, Lathyrus, Leontodon, Lilium, Linum, Lotus, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Solanum tuberosum, Sorbus, Spartium, Tecoma, Torenia, Tragopogon, Trollius, Tropaeolum, Tulipa, Tussilago, Ulex, Viola or Zinnia.

27. A process as claimed in claim 24, wherein the genetically modified plant used which, in comparison to the wild-type, has an increased β-cyclase activity in fruits, is a plant selected from the plant genera Actinophloeus, Aglaeonema, Ananas, Arbutus, Archontophoenix, Area, Aronia, Asparagus, Avocado, Attalea, Berberis, Bixia, Brachychilum, Bryonia, Caliptocalix, Capsicum, Carica, Celastrus, Citrullus, Citrus, Convallaria, Cotoneaster, Crataegus, Cucumis, Cucurbita, Cuscuta, Cycas, Cyphomandra, Dioscorea, Diospyrus, Dura, Elaeagnus, Elaeis, Erythroxylon, Euonymus, Erbse, Ficus, Fortunella, Fragaria, Gardinia, Gonocaryum, Gossypium, Guava, Guilielma, Hibiscus, Hippophaea, Iris, Kiwi, Lathyrus, Lonicera, Luffa, Lycium, Mais, Malpighia, Mangifera, Mormodica, Murraya, Musa, Nenga, Orange, Palisota, Pandanus, Passiflora, Persea, Physalis, Prunus, Ptychandra, Punica, Pyracantha, Pyrus, Ribes, Rosa, Rubus, Sabal, Sambucus, Seaforita, Shepherdia, Solanum, Sorbus, Synaspadix, Tabemae, Tamus, Taxus, Trichosanthes, Triphasia, Vaccinium, Viburnum, Vignia, Vitis or Zucchini.

28. A process as claimed in claim 24, wherein the genetically modified plant used which, in comparison to the wild-type, has an increased β-cyclase activity in tubers, is Solanum tuberosum.

29. A process as claimed in claim 1, wherein the β-carotenoids are selected from the group consisting of β-carotene, β-cryptoxanthin, zeaxanthin, antheraxanthin, violaxanthin and neoxanthin.

30. A genetically modified plant, the genetic modification increasing the activity of a β-cyclase in plant parts comprising photosynthetically inactive plastids, compared to the wild-type, and the increased β-cyclase activity being caused by a β-cyclase comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2.

31. A genetically modified plant as claimed in claim 30, wherein the increase in the β-cyclase activity is brought about by an increase in the gene expression of a nucleic acid encoding a β-cyclase comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, compared to the wild-type.

32. A genetically modified plant as claimed in claim 31, wherein, for the increase in the gene expression, nucleic acids are introduced into the plant which encode β-cyclases comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2.

33. A genetically modified plant comprising at least one nucleic acid encoding a β-cyclase comprising the amino acid sequence SEQ. ID. NO. 2 or a sequence derived from this sequence by substitution, insertion or deletion of amino acids, which has an identity of at least 60% at the amino acid level with the sequence SEQ. ID. NO. 2, with the proviso that tomato is excluded.

34. A genetically modified plant as claimed in claim 33, wherein the genetic modification additionally increases the hydroxylase activity compared to the wild-type.

35. A genetically modified plant as claimed in claim 33, wherein the genetic modification additionally reduces at least one of the activities selected from the group consisting of ε-cyclase activity and endogenous β-hydroxylase activity compared to the wild-type.

36. A genetically modified plant as claimed in claim 30, wherein the plant tissue comprising photosynthetically inactive plastids is selected from the group consisting of flower, fruit and tuber.

37. A genetically modified plant as claimed in claim 36, wherein the genetically modified plant which, in comparison to the wild-type, has an increased β-cyclase activity in flowers is selected from the families Ranunculaceae, Berberidaceae, Papaveraceae, Cannabaceae, Rosaceae, Fabaceae, Linaceae, Vitaceae, Brassiceae, Cucurbitaceae, Primulaceae, Caryophyllaceae, Amaranthaceae, Gentianaceae, Geraniaceae, Caprifoliaceae, Oleaceae, Tropaeolaceae, Solanaceae, Scrophulariaceae, Asteraceae, Liliaceae, Amaryllidaceae, Poaceae, Orchidaceae, Malvaceae, Illiaceae or Lamiaceae.

38. A genetically modified plant as claimed in claim 37, wherein the plant is selected from the plant genera Marigold, Tagetes erecta, Tagetes patula, Acacia, Aconitum, Adonis, Arnica, Aqulegia, Aster, Astragalus, Bignonia, Calendula, Calendula officinalis, Caltha, Campanula, Canna, Centaurea, Cheiranthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hisbiscus, Heliopsis, Hypericum, Hypochoeris, Impatiens, Iris, Jacaranda, Kerria, Laburnum, Lathyrus, Leontodon, Lilium, Linum, Lotus, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Solanum tuberosum, Sorbus, Spartium, Tecoma, Torenia, Tragopogon, Trollius, Tropaeolum, Tulipa, Tussilago, Ulex, Viola or Zinnia.

39. A genetically modified plant as claimed in claim 36, wherein the genetically modified plant which, in comparison to the wild-type, has an increased β-cyclase activity in fruits is selected from the plant genera Actinophloeus, Aglaeonema, Ananas, Arbutus, Archontophoenix, Area, Aronia, Asparagus, Avocado, Attalea, Berberis, Bixia, Brachychilum, Bryonia, Caliptocalix, Capsicum, Carica, Celastrus, Citrullus, Citrus, Convallaria, Cotoneaster, Crataegus, Cucumis, Cucurbita, Cuscuta, Cycas, Cyphomandra, Dioscorea, Diospyrus, Dura, Elaeagnus, Elaeis, Erythroxylon, Euonymus, Erbse, Ficus, Fortunella, Fragaria, Gardinia, Gonocaryum, Gossypium, Guava, Guilielma, Hibiscus, Hippophaea, Iris, Kiwi, Lathyrus, Lonicera, Luffa, Lycium, Mais, Malpighia, Mangifera, Mormodica, Murraya, Musa, Nenga, Orange, Palisota, Pandanus, Passiflora, Persea, Physalis, Prunus, Ptychandra, Punica, Pyracantha, Pyrus, Ribes, Rosa, Rubus, Sabal, Sambucus, Seaforita, Shepherdia, Solanum, Sorbus, Synaspadix, Tabemae, Tamus, Taxus, Trichosanthes, Triphasia, Vaccinium, Viburnum, Vignia, Vitis or Zucchini.

40. A genetically modified plant as claimed in claim 36, wherein the genetically modified plant which, in comparison to the wild-type, has an increased β-cyclase activity in tubers is Solanum tuberosum.

41. Foodstuffs or feedstuffs, which comprise of the genetically modified plants or plant tissues as claimed in claim 33.

42. A process for the production of γ-carotenoid-containing extracts or for the production of β-carotenoid-containing feed and food supplements, which comprises the use of the genetically modified plants or plant tissues as claimed in claim 33.

43. Pigmentation of animal products which comprises the zeaxanthin-containing extracts as set forth in claim 42.
